(19) United States
(12) Patent Application Publication
(10) Pub. No.: US 2025/0332151 A1
(43) Pub. Date: Oct. 30, 2025

(54) METHODS OF TREATMENT FOR CYSTIC FIBROSIS

(71) Applicant: Vertex Pharmaceuticals Incorporated, Boston, MA (US)

(72) Inventors: Bartlomiej BOREK, San Diego, CA (US); Julie LATERREUR, San Diego, CA (US); Jennifer LU, San Diego, CA (US); Valentin PRIETO CENTURION, Medford, MA (US); David RHEE, Needham, MA (US)

(21) Appl. No.: 18/864,603

(22) PCT Filed: May 15, 2023
(86) PCT No.: PCT/US2023/022276

§ 371 Date: Nov. 11, 2024

Related U.S. Application Data
(60) Provisional application No. 63/342,418, filed on May 16, 2022.

(51) Int. Cl. A61K 31/439; A61K 31/36; A61K 31/4155; A61K 31/416; A61K 31/4245; A61K 31/44; A61K 31/4439; A61K 31/4545; A61K 31/47; A61K 31/4709; A61K 31/4965; A61K 31/498; A61K 31/502; A61K 31/695; A61K 31/7048

(52) U.S. Cl. CPC A61K 31/439; A61K 31/36; A61K 31/4155; A61K 31/416; A61K 31/4245; A61K 31/44; A61K 31/4439; A61K 31/4545; A61K 31/47; A61K 31/4709; A61K 31/4965; A61K 31/498; A61K 31/502; A61K 31/695; A61K 31/7048

(57) ABSTRACT

This disclosure provides methods of treating cystic fibrosis or a CFTR-mediated disease comprising administering Compound I, a deuterated derivative thereof, or a pharmaceutically acceptable salt of any of the foregoing, e.g., administering about 1 mg to about 400 mg of Compound I. The disclosure also provides pharmaceutical compositions comprising Compound I, a deuterated derivative thereof, or a pharmaceutically acceptable salt of any of the foregoing, and optionally comprising one or more additional CFTR-modulating agents.

This application claims the benefit of U.S. Provisional Application No. 63/342,418, filed on May 16, 2022, the contents of which are incorporated by reference in their entirety.

The invention relates to modulators of Cystic Fibrosis Transmembrane Conductance Regulator (CFTR), pharmaceutical compositions containing the modulators, methods of treatment of cystic fibrosis and CFTR-mediated disorders using such modulators and pharmaceutical compositions, and processes for making such modulators.

Cystic fibrosis (CF) is a recessive genetic disease that affects approximately 88,000 children and adults worldwide. Despite progress in the treatment of CF, there is no cure.

In patients with CF, mutations in CFTR endogenously expressed in respiratory epithelia lead to reduced apical anion secretion causing an imbalance in ion and fluid transport. The resulting decrease in anion transport contributes to increased mucus accumulation in the lung and accompanying microbial infections that ultimately cause death in CF patients. In addition to respiratory disease, CF patients typically suffer from gastrointestinal problems and pancreatic insufficiency that, if left untreated, result in death. In addition, the majority of males with cystic fibrosis are infertile, and fertility is reduced among females with cystic fibrosis.

Sequence analysis of the CFTR gene has revealed a variety of disease-causing mutations (Cutting, G. R. et al. (1990) Nature 346:366-369; Dean, M. et al. (1990) Cell 61:863:870; and Kerem, B-S. et al. (1989) Science 245:1073-1080; Kerem, B-S. et al. (1990) Proc. Natl. Acad. Sci. USA 87:8447-8451). To date, greater than 2000 mutations in the CF gene have been identified; currently, the CFTR2 database contains information on at least 322 of these identified mutations, with sufficient evidence to define at least 281 mutations as disease-causing. The most prevalent disease-causing mutation is a deletion of phenylalanine at position 508 of the CFTR amino acid sequence and is commonly referred to as the F508del mutation. This mutation occurs in many of the cases of cystic fibrosis and is associated with severe disease.

CFTR is a cAMP/ATP-mediated anion channel that is expressed in a variety of cell types, including absorptive and secretory epithelia cells, where it regulates anion flux across the membrane, as well as the activity of other ion channels and proteins. In epithelial cells, normal functioning of CFTR is critical for the maintenance of electrolyte transport throughout the body, including respiratory and digestive tissue. CFTR is composed of 1480 amino acids that encode a protein which is made up of a tandem repeat of transmembrane domains, each containing six transmembrane helices and a nucleotide binding domain. The two transmembrane domains are linked by a large, polar, regulatory (R)-domain with multiple phosphorylation sites that regulate channel activity and cellular trafficking.

Chloride transport takes place by the coordinated activity of ENAC (epithelial sodium channel) and CFTR present on the apical membrane and the Na⁺-K⁺-ATPase pump and Cl⁻ channels expressed on the basolateral surface of the cell. Secondary active transport of chloride from the luminal side leads to the accumulation of intracellular chloride, which can then passively leave the cell via Cl-channels, resulting in a vectorial transport. Arrangement of Na⁺/2Cl⁻/K⁺ co-transporter, Na⁺-K⁺-ATPase pump and the basolateral membrane K⁺ channels on the basolateral surface and CFTR on the luminal side coordinate the secretion of chloride. Because water is probably never actively transported itself, its flow across epithelia depends on tiny transepithelial osmotic gradients generated by the bulk flow of sodium and chloride.

A number of CFTR modulators have recently been identified. These modulators can be characterized as, for example, potentiators, correctors, potentiator enhancers/co-potentiators, amplifiers, readthrough agents, and nucleic acid therapies. CFTR modulators that increase the channel gating activity of mutant and wild-type CFTR at the epithelial cell surface are known as potentiators. Correctors improve faulty protein processing and resulting trafficking to the epithelial surface. Ghelani and Schneider-Futschik (2020) ACS Pharmacol. Transl. Sci. 3:4-10. There are three CFTR correctors approved by the U.S. FDA for treatment of cystic fibrosis. However, monotherapy with some CFTR correctors has not been found to be effective enough and as a result combination therapy with a potentiator is often needed to enhance CFTR activity. There is currently only one CFTR potentiator that is approved for the treatment of cystic fibrosis. Accordingly, there is a need for novel treatments of cystic fibrosis and other CFTR mediated diseases.

Thus, one aspect of the disclosure provides a CFTR-modulating compound, (6R)-17-amino-12,12-dimethyl-6,15-bis(trifluoromethyl)-19-oxa-3,4,13,18-tetrazatricyclo[12.3.1.12,5]nonadeca-1 (18),2,4,14,16-pentaen-6-ol (Compound I), deuterated derivatives thereof, and pharmaceutically acceptable salts of any of the foregoing. Compound I can be depicted as having the following structure:

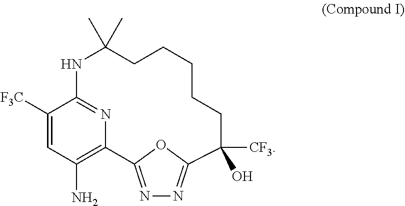 [SMILES: CC1(C)CCCCC[C@](O)(C(F)(F)F)C2=NN=C(O2)c2nc(c(C(F)(F)F)cc2N)N1]

Other aspects of the disclosure provide pharmaceutical compositions comprising Compound I, a deuterated derivative thereof, or a pharmaceutically acceptable salt of any of the foregoing, which compositions may further include at least one additional active pharmaceutical ingredient and/or at least one pharmaceutically acceptable carrier. Yet other aspects of the disclosure are methods of treating the CFTR-mediated disease cystic fibrosis comprising administering Compound I, a deuterated derivative thereof, or a pharmaceutically acceptable salt of any of the foregoing, optionally as part of a pharmaceutical composition comprising at least one additional component, to a subject in need thereof.

Another aspect of the invention provides methods of treating the CFTR-mediated disease cystic fibrosis comprising administering to a patient in need thereof Compound I, a deuterated derivative thereof, or a pharmaceutically acceptable salt of any of the foregoing, alone or in combination with one or more additional CFTR-modulating agents selected from (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl) cyclopropanecarboxamide (Compound II), N-[2,4-bis(1,1-dimethylethyl)-5-hydroxyphenyl]-1,4-dihydro-4-oxoquinoline-3-carboxamide (also known as N-[2,4-bis(1,1-dimethylethyl)-5-hydroxyphenyl]-1,4-dihydro-4-oxoquinoline-3-carboxamide) (Compound III) or N-(2-(tert-butyl)-5-hydroxy-4-(2-(methyl-d3) propan-2-yl-1,1,1,3,3,3-d6)phenyl)-4-oxo-1,4-dihydroquinoline-3-carboxamide (Compound III-d), 3-(6-(1-(2,2-difluorobenzo[d][1,3]dioxol-5-yl) cyclopropane carboxamido)-3-methylpyridin-2-yl)benzoic acid (Compound IV), N-(1,3-dimethylpyrazol-4-yl)sulfonyl-6-[3-(3,3,3-trifluoro-2,2-dimethyl-propoxy) pyrazol-1-yl]-2-[(4S)-2,2,4-trimethylpyrrolidin-1-yl]pyridine-3-carboxamide (Compound V), N-(benzenesulfonyl)-6-[3-[2-[1-(trifluoromethyl) cyclopropyl]ethoxy]pyrazol-1-yl]-2-[(4S)-2,2,4-trimethylpyrrolidin-1-yl]pyridine-3-carboxamide (Compound VI), (14S)-8-[3-(2-{dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1 (22),5,7,9,19 (23),20-hexaene-2,2,4-trione (Compound VII), (11R)-6-(2,6-dimethylphenyl)-11-(2-methylpropyl)-12-{spiro[2.3]hexan-5-yl}-9-oxa-27.6-thia-3,5,12,19-tetraazatricyclo[12.3.1.14,8]nonadeca-1 (17),4 (19),5,7,14 (18), 15-hexaene-2,2,13-trione (Compound VIII); N-(benzenesulfonyl)-6-(3-fluoro-5-isobutoxy-phenyl)-2-[(4S)-2,2,4-trimethylpyrrolidin-1-yl]pyridine-3-carboxamide (Compound IX), and N-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide (Compound X). In some embodiments, Compound I, a deuterated derivative thereof, or a pharmaceutically acceptable salt of any of the foregoing, is administered in the same composition with at least one compound chosen from Compound II, Compound III, Compound III-d, Compound IV, Compound V, Compound VI, Compound VII, Compound VIII, Compound IX, Compound X, deuterated derivatives thereof, and pharmaceutically acceptable salts of any of the foregoing. In some embodiments, a composition comprising Compound I, a deuterated derivative thereof, or a pharmaceutically acceptable salt of any of the foregoing, is co-administered with a separate composition comprising at least one compound chosen from Compound II, Compound III, Compound III-d, Compound IV, Compound V, Compound VI, Compound VII, Compound VIII, Compound IX, Compound X, deuterated derivatives thereof, and pharmaceutically acceptable salts of any of the foregoing.

Another aspect of the invention provides methods of treating the CFTR-mediated disease cystic fibrosis comprising administering to a patient in need thereof Compound I, a deuterated derivative thereof, or a pharmaceutically acceptable salt of any of the foregoing, and optionally further administering one or more additional CFTR-modulating agents selected from ASP-11, disclosed in Journal of Cystic Fibrosis (2018), 17 (5), 595-606, and nesolicaftor or PTI-428, disclosed in WO 2016/105485. In one embodiment, the additional CFTR-modulating agent is ASP-11. In one embodiment, the additional CFTR-modulating agent comprises PTI-428.

Another aspect of the invention provides methods of treating the CFTR-mediated disease cystic fibrosis comprising administering to a patient in need thereof Compound I, a deuterated derivative thereof, or a pharmaceutically acceptable salt of any of the foregoing, and optionally further administering one or more additional CFTR-modulating agents selected from galicaftor or ABBV-2222, disclosed in United States Patent Application Publication No. 2016-0120841; ABBV-3221, disclosed in WO 2018/065921; posenacaftor or PTI-801, disclosed in WO 2017/062581; ABBV-2851, disclosed in WO 2017/009804; GLPG2737, disclosed in United States Patent Application Publication No. 2017-0101405; ABBV-3748; ABBV-3903; and ABBV-119.

In certain embodiments, the pharmaceutical compositions of the invention comprise Compound I, a deuterated derivative thereof, or a pharmaceutically acceptable salt of any of the foregoing, and may optionally further comprise at least one compound chosen from Compound II, Compound III, Compound III-d, Compound IV, Compound V, Compound VI, Compound VII, Compound VIII, Compound IX, Compound X, deuterated derivatives thereof, and pharmaceutically acceptable salts of any of the foregoing.

### BRIEF DESCRIPTION OF THE FIGURES

FIG. 1 provides an X-ray power diffraction (XRPD) pattern of crystalline Compound I Form A (neat).

FIG. 2 provides a differential scanning calorimetry (DSC) analysis of crystalline Compound I Form A (neat).

FIG. 3 provides a Study Design for Part A of a first-in-human (FIH) study of Compound I and provides the doses that were studied.

FIG. 4 provides a Study Design for Part B of a FIH study of Compound I and provides the doses that were studied.

### DEFINITIONS

“Compound I” as used throughout this disclosure refers to (6R)-17-amino-12,12-dimethyl-6,15-bis(trifluoromethyl)-19-oxa-3,4,13,18-tetrazatricyclo[12.3.1.12,5]nonadeca-1 (18),2,4,14,16-pentaen-6-ol, which can be depicted as having the following structure:

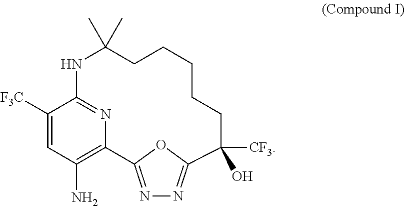 [SMILES: CC1(C)CCCCC[C@](O)(C(F)(F)F)C2=NN=C(O2)c2nc(c(C(F)(F)F)cc2N)N1]

Compound I may be a racemic mixture or an enantioenriched (e.g., >90% ee, >95% ee, >98% ee) mixture of isomers. Compound I or a deuterated derivative thereof may be in the form of a pharmaceutically acceptable salt, solvate, and/or hydrate. Compound I and methods for making and using Compound I are disclosed in PCT International Application No. PCT/US2021/072475, incorporated herein by reference.

“Compound II,” as used herein, refers to (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl) cyclopropanecarboxamide, which can be depicted with the following structure:

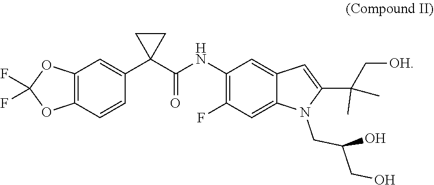 [SMILES: CC(C)(CO)c1cc2cc(NC(=O)C3(c4ccc5c(c4)OC(F)(F)O5)CC3)c(F)cc2n1C[C@@H](O)CO]

Compound II may be in the form of a pharmaceutically acceptable salt. Compound II and methods of making and using Compound II are disclosed in WO 2010/053471, WO 2011/119984, WO 2011/133751, WO 2011/133951, and WO 2015/160787, each incorporated herein by reference.

“Compound III” as used throughout this disclosure refers to N-(5-hydroxy-2,4-di-tert-butyl-phenyl)-4-oxo-1H-quinoline-3-carboxamide (also known as N-[2,4-bis(1,1-dimethylethyl)-5-hydroxyphenyl]-1,4-dihydro-4-oxoquinoline-3-carboxamide), which is depicted by the structure:

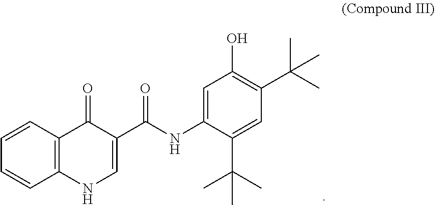 [SMILES: CC(C)(C)c1cc(C(C)(C)C)c(NC(=O)c2c[nH]c3ccccc3c2=O)cc1O]

Compound III may also be in the form of a pharmaceutically acceptable salt. Compound III and methods of making and using Compound III are disclosed in WO 2006/002421, WO 2007/079139, WO 2010/108162, and WO 2010/019239, each incorporated herein by reference.

In some embodiments, a deuterated derivative of Compound III (Compound III-d) is employed in the compositions and methods disclosed herein. A chemical name for Compound III-d is N-(2-(tert-butyl)-5-hydroxy-4-(2-(methyl-d3) propan-2-yl-1,1,1,3,3,3-d6)phenyl)-4-oxo-1,4-dihydroquinoline-3-carboxamide, as depicted by the structure:

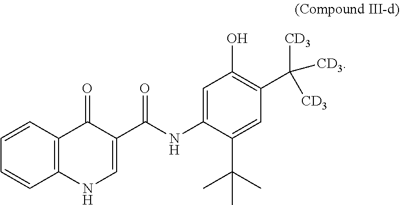 [SMILES: [2H]C([2H])([2H])C(c1cc(C(C)(C)C)c(NC(=O)c2c[nH]c3ccccc3c2=O)cc1O)(C([2H])([2H])[2H])C([2H])([2H])[2H]]

Compound III-d may be in the form of a pharmaceutically acceptable salt. Compound III-d and methods of making and using Compound III-d are disclosed in WO 2012/158885, WO 2014/078842, WO 2019/109021, and U.S. Pat. No. 8,865,902, incorporated herein by reference.

“Compound IV” as used herein, refers to 3-(6-(1-(2,2-difluorobenzo[d][1,3]dioxol-5-yl) cyclopropanecarboxamido)-3-methylpyridin-2-yl)benzoic acid, which is depicted by the chemical structure:

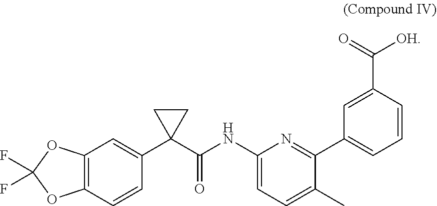 [SMILES: Cc1ccc(NC(=O)C2(c3ccc4c(c3)OC(F)(F)O4)CC2)nc1-c1cccc(C(=O)O)c1]

Compound IV may be in the form of a pharmaceutically acceptable salt. Compound IV and methods of making and using Compound IV are disclosed in WO 2007/056341, WO 2009/073757, and WO 2009/076142, incorporated herein by reference.

“Compound V” as used herein, refers to N-(1,3-dimethylpyrazol-4-yl)sulfonyl-6-[3-(3,3,3-trifluoro-2,2-dimethyl-propoxy) pyrazol-1-yl]-2-[(4S)-2,2,4-trimethylpyrrolidin-1-yl]pyridine-3-carboxamide, which is depicted by the chemical structure:

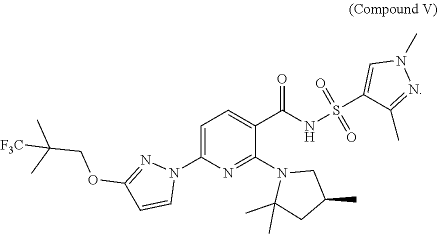 [SMILES: Cc1nn(C)cc1S(=O)(=O)NC(=O)c1ccc(-n2ccc(OCC(C)(C)C(F)(F)F)n2)nc1N1C[C@@H](C)CC1(C)C]

Compound V may be in the form of a pharmaceutically acceptable salt. Compound V and methods of making and using Compound V are disclosed in WO 2018/107100 and WO 2019/113476, incorporated herein by reference.

“Compound VI” as used herein, refers to N-(benzenesulfonyl)-6-[3-[2-[1-(trifluoromethyl)cyclopropyl]ethoxy]pyrazol-1-yl]-2-[(4S)-2,2,4-trimethylpyrrolidin-1-yl]pyridine-3-carboxamide, which is depicted by the chemical structure:

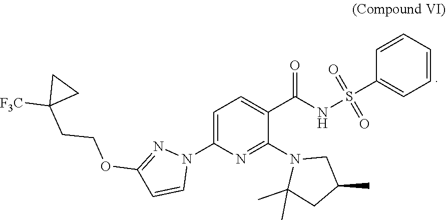 [SMILES: C[C@@H]1CN(c2nc(-n3ccc(OCCC4(C(F)(F)F)CC4)n3)ccc2C(=O)NS(=O)(=O)c2ccccc2)C(C)(C)C1]

Compound VI may be in the form of a pharmaceutically acceptable salt. Compound VI and methods of making and using Compound VI are disclosed in WO 2018/064632 and WO 2019/113476, incorporated herein by reference.

“Compound VII” as used herein, refers to (14S)-8-[3-(2-{dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1 (22),5,7,9,19 (23),20-hexaene-2,2,4-trione, which is depicted by the chemical structure:

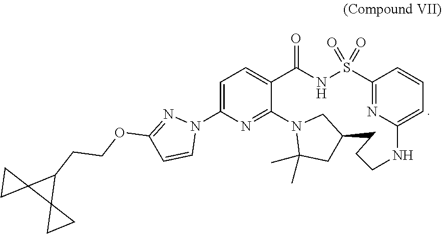 [SMILES: CC1(C)C[C@@H]2CCCNc3cccc(n3)S(=O)(=O)NC(=O)c3ccc(-n4ccc(OCCC5C6(CC6)C56CC6)n4)nc3N1C2]

Compound VII may be in the form of a pharmaceutically acceptable salt. Compound VII and methods of making and using Compound VII are disclosed in WO 2019/161078, WO 2020/102346, and WO 2021/030554, incorporated herein by reference.

“Compound VIII” as used herein, refers to (11R)-6-(2,6-dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1 (17),4 (19),5,7,14 (18), 15-hexaene-2,2,13-trione, which is depicted by the chemical structure:

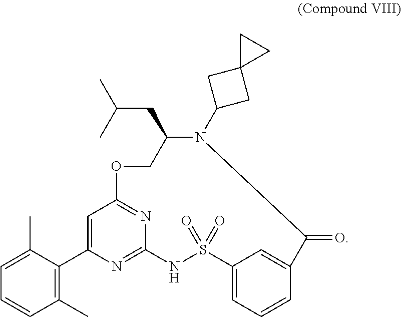 [SMILES: Cc1cccc(C)c1-c1cc2nc(n1)NS(=O)(=O)c1cccc(c1)C(=O)N(C1CC3(CC3)C1)[C@H](CC(C)C)CO2]

Compound VIII may be in the form of a pharmaceutically acceptable salt. Compound VIII and methods of making and using Compound VIII are disclosed in WO 2020/206080, incorporated herein by reference.

“Compound IX” as used herein, refers to N-(benzenesulfonyl)-6-(3-fluoro-5-isobutoxy-phenyl)-2-[(4S)-2,2,4-trimethylpyrrolidin-1-yl]pyridine-3-carboxamide, which is depicted by the chemical structure:

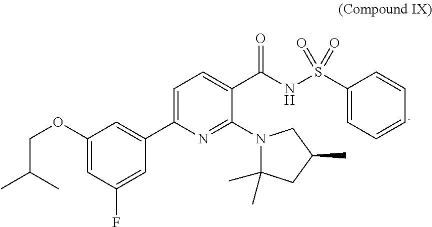 [SMILES: CC(C)COc1cc(F)cc(-c2ccc(C(=O)NS(=O)(=O)c3ccccc3)c(N3C[C@@H](C)CC3(C)C)n2)c1]

Compound IX may be in the form of a pharmaceutically acceptable salt. Compound IX and methods of making and using Compound IX are disclosed in WO 2016/057572, incorporated herein by reference.

“Compound X” as used herein, refers to N-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide, which is depicted by the chemical structure:

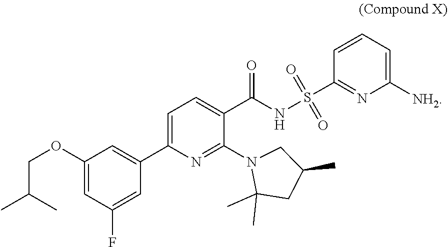 [SMILES: CC(C)COc1cc(F)cc(-c2ccc(C(=O)NS(=O)(=O)c3cccc(N)n3)c(N3C[C@@H](C)CC3(C)C)n2)c1]

Compound X may be in the form of a pharmaceutically acceptable salt. Compound X and methods of making and using Compound X are disclosed in WO 2016/057572, incorporated herein by reference.

As used herein, “CFTR” means cystic fibrosis transmembrane conductance regulator.

As used herein, “mutations” can refer to mutations in the CFTR gene or the CFTR protein. A “CFTR gene mutation” refers to a mutation in the CFTR gene, and a “CFTR protein mutation” refers to a mutation in the CFTR protein. A genetic defect or mutation (i.e., a change in one or more nucleotides) in a gene in general results in a mutation (i.e., a change in one or more amino acids) in the CFTR protein translated from that gene, or a frame shift (i.e., a change in the reading frame that governs how the CFTR gene is translated into the CFTR protein).

As used herein, the term “F508del” refers to a mutant CFTR protein which is lacking the amino acid phenylalanine at position 508, or to a mutant CFTR gene which encodes for a CFTR protein lacking the amino acid phenylalanine at position 508.

As used herein, a patient who is “homozygous” for a particular gene mutation has the same mutation on each allele.

As used herein, a patient who is “heterozygous” for a particular gene mutation has this mutation on one allele, and a different mutation on the other allele.

As used herein, the terms “CFTR modulator,” “CFTR-modulating compound,” and “CFTR-modulating agent” interchangeably refer to a compound that directly or indirectly increases the activity of CFTR. CFTR modulators include but are not limited to compounds that correct, potentiate, stabilize, and/or amplify CFTR.

As used herein, the term “CFTR corrector” refers to a compound that facilitates the processing and trafficking of CFTR to increase the amount of CFTR at the cell surface. Compound II, Compound IV, Compound V, Compound VI, Compound VII, Compound VIII, Compound IX, and Compound X disclosed herein are CFTR correctors.

As used herein, the term “CFTR potentiator” refers to a compound that increases the channel activity of CFTR protein located at the cell surface, resulting in enhanced ion transport. Compound III and Compound III-d disclosed herein are CFTR potentiators.

As used herein, the term “active pharmaceutical ingredient” (“API”) or “therapeutic agent” refers to a biologically active compound.

It will be appreciated that certain compounds of this invention may exist as separate stereoisomers or enantiomers and/or mixtures of those stereoisomers or enantiomers. As used in the chemical structures disclosed herein, a “wedge”( [symbol] ) or “hash” ( [symbol] ) bond to a stereogenic atom indicates a chiral center of known absolute stereochemistry (i.e., one stereoisomer). As used in the chemical structures disclosed herein, a “wavy” bond ( [symbol] ) to a stereogenic atom indicates a chiral center of unknown absolute stereochemistry (i.e., one stereoisomer). As used in the chemical structures disclosed herein, a “wavy” bond ( [symbol] ) to a double-bonded carbon indicates a mixture of E/Z isomers. As used in the chemical structures disclosed herein, a [symbol] (“straight”) bond to a stereogenic atom indicates where there is a mixture (e.g., a racemate or enrichment). As used herein, two [symbol] (“straight”) bonds to a double-bonded carbon indicates that the double bond possesses the E/Z stereochemistry as drawn. As used in the chemical structures disclosed herein, a

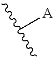

(i.e., a “wavy” line perpendicular to a “straight” bond to group “A”) indicates that group “A” is a substituent whose point of attachment is at the end of the bond that terminates at the “wavy” line.

The terms “patient” and “subject” are used interchangeably and refer to an animal including humans.

The terms “effective dose” and “effective amount” are used interchangeably herein and refer to that amount of a compound that produces the desired effect for which it is administered (e.g., improvement in CF or a symptom of CF, or lessening the severity of CF or a symptom of CF). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, the terms “treatment,” “treating,” and the like generally mean the improvement of one or more symptoms of CF in a patient diagnosed with the disease, a lessening the severity of CF or one or more of its symptoms in a subject, or a slowing of the progression of the disease in a subject. “Treatment,” as used herein, includes, but is not limited to, the following: increased growth of the subject, increased weight gain, reduction of mucus in the lungs, improved pancreatic and/or liver function, reduction of chest infections, and/or reductions in coughing or shortness of breath. Improvements in, lessening the severity of any of these symptoms, or slowing the progression of the disease can be readily assessed according to standard methods and techniques known in the art.

As used herein, the term “in combination with,” when referring to two or more compounds, agents, or additional APIs, means the administration of two or more compounds, agents, or APIs to the patient prior to, concurrently with, or subsequent to each other.

The terms “about” and “approximately,” when used in connection with doses, amounts, or weight percentages of ingredients of a composition or a dosage form, include the value of a specified dose, amount, or weight percentage or a range of the dose, amount, or weight percentage that is recognized by one of ordinary skill in the art to provide a pharmacological effect equivalent to that obtained from the specified dose, amount, or weight percent. The terms “about” and “approximately” may refer to an acceptable error for a particular value as determined by one of skill in the art, which depends in part on how the values is measured or determined. In some embodiments, the terms “about” and “approximately” mean within 15%, 10%, 5%, 4%, 3%, 2%, 1%, or 0.5% of a given value or range. In some embodiments, the terms “about” and “approximately” mean within 15% of a given value. In some embodiments, the terms “about” and “approximately” mean within 10% of a given value.

As used herein, “minimal function (MF) mutations” refer to CFTR gene mutations associated with minimal CFTR function (little-to-no functioning CFTR protein) and include, for example, mutations associated with severe defects in ability of the CFTR channel to open and close, known as defective channel gating or “gating mutations”; mutations associated with severe defects in the cellular processing of CFTR and its delivery to the cell surface; mutations associated with no (or minimal) CFTR synthesis; and mutations associated with severe defects in channel conductance.

As used herein, “deuterated derivative(s)” means the same chemical structure, with one or more hydrogen atoms replaced by a deuterium atom.

As used herein, the term “pharmaceutically acceptable salt” refers to a salt form of a compound of this disclosure, or a deuterated derivative thereof, wherein the salt is nontoxic. Pharmaceutically acceptable salts of the compounds of this disclosure include those derived from suitable inorganic and organic acids and bases. A “free base” form of a compound, for example, does not contain an ionically bonded salt.

The phrase “deuterated derivative or pharmaceutically acceptable salt thereof” is used interchangeably with “deuterated derivative thereof or pharmaceutically acceptable salt of any of the forgoing” in reference to one or more compounds or formulae of the invention. The phrase “pharmaceutically acceptable salt and deuterated derivative thereof” is used interchangeably with “pharmaceutically acceptable salt thereof and deuterated derivative of any of the forgoing” in reference to one or more compounds or formulae of the invention. These phrases are intended to encompass pharmaceutically acceptable salts of any one of the referenced compounds, deuterated derivatives of any one of the referenced compounds, deuterated derivatives of pharmaceutically acceptable salts of any one of the referenced compounds, and pharmaceutically acceptable salts of deuterated derivatives of any one of the referenced compounds.

One of ordinary skill in the art would recognize that, when an amount of “a compound or a pharmaceutically acceptable salt thereof” is disclosed, the amount of the pharmaceutically acceptable salt form of the compound is the amount equivalent to the concentration of the free base of the compound. It is noted that the disclosed amounts of the compounds or their pharmaceutically acceptable salts thereof herein are based upon their free base form. For example, “100 mg of Compound I or a pharmaceutically acceptable salt thereof” or “100 mg of Compound I or an equivalent amount of a pharmaceutically acceptable salt thereof” includes 100 mg of Compound I and an amount of a pharmaceutically acceptable salt of Compound I equivalent to 100 mg of Compound I.

Suitable pharmaceutically acceptable salts are, for example, those disclosed in S. M. Berge, et al. J. Pharmaceutical Sciences, 1977, 66, 1-19. For example, Table 1 of that article provides the following pharmaceutically acceptable salts:

TABLE 1

| | | |
|---|---|---|
| Acetate | Iodide | Benzathine |
| Benzenesulfonate | Isethionate | Chloroprocaine |
| Benzoate | Lactate | Choline |
| Bicarbonate | Lactobionate | Diethanolamine |
| Bitartrate | Malate | Ethylenediamine |
| Bromide | Maleate | Meglumine |
| Calcium edetate | Mandelate | Procaine |
| Camsylate | Mesylate | Aluminum |
| Carbonate | Methylbromide | Calcium |
| Chloride | Methylnitrate | Lithium |
| Citrate | Methylsulfate | Magnesium |
| Dihydrochloride | Mucate | Potassium |
| Edetate | Napsylate | Sodium |
| Edisylate | Nitrate | Zinc |
| Estolate | Pamoate (Embonate) | |
| Esylate | Pantothenate | |
| Fumarate | Phosphate/diphosphate | |
| Gluceptate | Polygalacturonate | |
| Gluconate | Salicylate | |
| Glutamate | Stearate | |
| Glycollylarsanilate | Subacetate | |
| Hexylresorcinate | Succinate | |
| Hydrabamine | Sulfate | |
| Hydrobromide | Tannate | |
| Hydrochloride | Tartrate | |
| Hydroxynaphthoate | Teociate | |
| | Triethiodide | |

Non-limiting examples of pharmaceutically acceptable acid addition salts include: salts formed with inorganic acids, such as hydrochloric acid, hydrobromic acid, phosphoric acid, sulfuric acid, or perchloric acid; salts formed with organic acids, such as acetic acid, oxalic acid, maleic acid, tartaric acid, citric acid, succinic acid or malonic acid; and salts formed by using other methods used in the art, such as ion exchange. Non-limiting examples of pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. Pharmaceutically acceptable salts derived from appropriate bases include alkali metal, alkaline earth metal, ammonium, and N⁺ (C₁₋₄alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate, and aryl sulfonate. Other suitable, non-limiting examples of pharmaceutically acceptable salts include besylate and glucosamine salts.

As used herein, the term “XRPD” refers to the analytical characterization method of X-ray powder diffraction. XRPD patterns disclosed herein were recorded at ambient conditions in transmission or reflection geometry using a diffractometer.

As used herein, the terms “X-ray powder diffractogram,” “X-ray powder diffraction pattern,” “XRPD pattern,” “XRPD spectrum” interchangeably refer to an experimentally obtained pattern plotting signal positions (on the abscissa) versus signal intensities (on the ordinate). For an amorphous material, an X-ray powder diffractogram may include one or more broad signals; and for a crystalline material, an X-ray powder diffractogram may include one or more signals, each identified by its angular value as measured in degrees 2θ (° 20), depicted on the abscissa of an X-ray powder diffractogram, which may be expressed as “a signal at . . . degrees two-theta,” “a signal at [a]two-theta value(s) of . . . ” and/or “a signal at at least . . . two-theta value(s) selected from . . . ”

As used herein, the term “DSC” refers to the analytical method of Differential Scanning calorimetry.

As used herein, the term “melting temperature”, “melting point”, or “Tm” refers to the temperature at which a material transitions from a solid to a liquid phase.

### Detailed Description of Embodiments

### Methods of Treatment

Compound I, a deuterated derivative thereof, or a pharmaceutically acceptable salt of any of the foregoing, can act as a CFTR modulator, i.e., it modulates CFTR activity in the body. Individuals suffering from a mutation in the gene encoding CFTR may benefit from receiving a CFTR modulator. A CFTR mutation may affect CFTR quantity, i.e., the number of CFTR channels at the cell surface, or it may impact CFTR function, i.e., the functional ability of each channel to open and transport ions. Mutations affecting CFTR quantity include mutations that cause defective synthesis (Class I defect), mutations that cause defective processing and trafficking (Class II defect), mutations that cause reduced synthesis of CFTR (Class V defect), and mutations that reduce the surface stability of CFTR (Class VI defect). Mutations that affect CFTR function include mutations that cause defective gating (Class III defect) and mutations that cause defective conductance (Class IV defect). Some CFTR mutations exhibit characteristics of multiple classes. Certain mutations in the CFTR gene result in CF.

Thus, in some embodiments, the invention provides methods of treating, lessening the severity of, or symptomatically treating cystic fibrosis in a patient comprising administering to the patient an effective amount of Compound I, a deuterated derivative thereof, or a pharmaceutically acceptable salt of any of the foregoing, alone or in combination with another active ingredient, such as another CFTR-modulating agent. In some embodiments, the patient has an F508del/minimal function (MF) genotype, F508del/F508del genotype (homozygous for the F508del mutation), F508del/gating genotype, or F508del/residual function (RF) genotype. In some embodiments, the patient is heterozygous and has one F508del mutation. In some embodiments, the patient is homozygous for the F508del mutation. In some embodiments, the patient is homozygous for the N1303K mutation.

In some embodiments, about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, is administered daily.

In some embodiments, about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, is administered daily . . .

In some embodiments, about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, is administered as a single dose.

In some embodiments, a total daily dose of about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, is administered as a single dose.

In some embodiments, about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, is administered in two doses.

In some embodiments, the total daily dose of about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, is administered in two doses.

In some embodiments, the patient is heterozygous and has an F508del mutation on one allele and a mutation on the other allele selected from Table 2:

TABLE 2
CFTR Mutations
Mutation

| | | | | |
|---|---|---|---|---|
| Q2X | L218X | Q525X | R792X | E1104X |
| S4X | Q220X | G542X | E822X | W1145X |
| W19X | Y275X | G550X | W882X | R1158X |
| G27X | C276X | Q552X | W846X | R1162X |
| Q39X | Q290X | R553X | Y849X | S1196X |
| W57X | G330X | E585X | R851X | W1204X |
| E60X | W401X | G673X | Q890X | L1254X |
| R75X | Q414X | Q685X | S912X | S1255X |
| L88X | S434X | R709X | Y913X | W1282X |
| E92X | S466X | K710X | Q1042X | Q1313X |
| Q98X | S489X | Q715X | W1089X | Q1330X |
| Y122X | Q493X | L732X | Y1092X | E1371X |
| E193X | W496X | R764X | W1098X | Q1382X |
| W216X | C524X | R785X | R1102X | Q1411X |
| 185 + 1G→T | 711 + 5G→A | 1717 − 8G→A | 2622 + 1G→A | 3121 − 1G→A |
| 296 + 1G→A | 712 − 1G→T | 1717 − 1G→A | 2790 − 1G→C | 3500 − 2A→G |
| 296 + 1G→T | 1248 + 1G→A | 1811 + 1G→C | 3040G→C | 3600 + 2insT |
| 405 + 1G→A | 1249 − 1G→A | 1811 + 1.6kbA→G | (G970R) | 3850 − 1G→A |
| 405 + 3A→C | 1341 + 1G→A | 1811 + 1643G→T | 3120G→A | 4005 + 1G→A |
| 406 − 1G→A | 1525 − 2A→G | 1812 − 1G→A | 3120 + 1G→A | 4374 + 1G→T |
| 621 + 1G→T | 1525 − 1G→A | 1898 + 1G→A | 3121 − 2A→G | |
| 711 + 1G→T | | 1898 + 1G→C | | |
| 182delT | 1078delT | 1677delTA | 2711delT | 3737delA |
| 306insA | 1119delA | 1782delA | 2732insA | 3791delC |
| 306delTAGA | 1138insG | 1824delA | 2869insG | 3821delT |
| 365-366insT | 1154insTC | 1833delT | 2896insAG | 3876delA |
| 394delTT | 1161delC | 2043delG | 2942insT | 3878delG |
| 442delA | 1213delT | 2143delT | 2957delT | 3905insT |
| 444delA | 1259insA | 2183AA→G | 3007delG | 4016insT |
| 457TAT→G | 1288insTA | 2184delA | 3028delA | 4021dupT |
| 541delC | 1343delG | 2184insA | 3171delC | 4022insT |
| 574delA | 1471delA | 2307insA | 3171insC | 4040delA |
| 663delT | 1497delGG | 2347delG | 3271delGG | 4279insA |
| 849delG | 1548delG | 2585delT | 3349insT | 4326delTC |
| 935delA | 1609del CA | 2594delGT | 3659delC | |

| | | |
|---|---|---|
| CFTRdele1 | CFTRdele16-17b | 1461ins4 |
| CFTRdele2 | CFTRdele17a, 17b | 1924del7 |
| CFTRdele2, 3 | CFTRdele17a-18 | 2055del9→A |
| CFTRdele2-4 | CFTRdele19 | 2105-2117del13insAGAAA |
| CFTRdele3-10, 14b-16 | CFTRdele19-21 | 2372del8 |
| CFTRdele4-7 | CFTRdele21 | 2721del11 |
| CFTRdele4-11 | CFTRdele22-24 | 2991del32 |
| CFTR50kbdel | CFTRdele22, 23 | 3667ins4 |
| CFTRdup6b-10 | 124del23bp | 4010del4 |
| CFTRdele11 | 602del14 | 4209TGTT→AA |
| CFTRdele13, 14a | 852del22 | |
| CFTRdele14b-17b | 991del5 | |

| | | | |
|---|---|---|---|
| A46D | V520F | Y569D | N1303K |
| G85E | A559T | L1065P | |
| R347P | R560T | R1066C | |
| L467P | R560S | L1077P | |
| I507del | A561E | M1101K | |

In some embodiments, the disclosure also is directed to methods of treatment using isotope-labelled compounds of the afore-mentioned compounds or pharmaceutically acceptable salts thereof, wherein the formula and variables of such compounds and salts are each and independently as described above or any other embodiments described above, provided that one or more atoms therein have been replaced by an atom or atoms having an atomic mass or mass number which differs from the atomic mass or mass number of the atom which usually occurs naturally (isotope-labelled). Examples of isotopes which are commercially available and suitable for the disclosure include isotopes of hydrogen, carbon, nitrogen, oxygen, phosphorus, fluorine and chlorine, for example ²H, ³H, ¹³C, ¹⁴C, ¹⁵N, ¹⁸O, ¹⁷O, ³¹P, ³²p, ³⁵S, ¹⁸F and 36Cl, respectively.

The isotope-labelled compounds and salts can be used in a number of beneficial ways. They can be suitable for medicaments and/or various types of assays, such as substrate tissue distribution assays. For example, tritium (³H)- and/or carbon-14 (¹⁴C)-labelled compounds are particularly useful for various types of assays, such as substrate tissue distribution assays, due to relatively simple preparation and excellent detectability. For example, deuterium (²H)-labelled ones are therapeutically useful with potential therapeutic advantages over the non-²H-labelled compounds. In general, deuterium (²H)-labelled compounds and salts can have higher metabolic stability as compared to those that are not isotope-labelled owing to the kinetic isotope effect described below. Higher metabolic stability translates directly into an increased in vivo half-life or lower dosages, which could be desired. The isotope-labelled compounds and salts can usually be prepared by carrying out the procedures disclosed in the synthesis schemes and the related description, in the example part and in the preparation part in the present text, replacing a non-isotope-labelled reactant by a readily available isotope-labelled reactant.

In some embodiments, the isotope-labelled compounds and salts are deuterium (²H)-labelled ones. In some specific embodiments, the isotope-labelled compounds and salts are deuterium (²H)-labelled, wherein one or more hydrogen atoms therein have been replaced by deuterium. In chemical structures, deuterium is represented as “²H” or “D.”

When discovering and developing therapeutic agents, the person skilled in the art attempts to optimize pharmacokinetic parameters while retaining desirable in vitro properties. It may be reasonable to assume that many compounds with poor pharmacokinetic profiles are susceptible to oxidative metabolism.

The deuterium (²H)-labelled compounds and salts can modulate the oxidative metabolism of the compound by way of the primary kinetic isotope effect. The primary kinetic isotope effect is a change of the rate for a chemical reaction that results from exchange of isotopic nuclei, which in turn is caused by the change in ground state energies necessary for covalent bond formation after this isotopic exchange. Exchange of a heavier isotope usually results in a lowering of the ground state energy for a chemical bond and thus causes a reduction in the rate-limiting bond breakage. If the bond breakage occurs in or in the vicinity of a saddle-point region along the coordinate of a multi-product reaction, the product distribution ratios can be altered substantially. For explanation: if deuterium is bonded to a carbon atom at a non-exchangeable position, rate differences of k_{M}k_{D}=2-7 are typical. For a further discussion, see S. L. Harbeson and R. D. Tung, Deuterium In Drug Discovery and Development, Ann. Rep. Med. Chem. 2011, 46, 403-417, which is incorporated herein by reference.

The concentration of the isotope(s) (e.g., deuterium) incorporated into the isotope-labelled compounds and salt of the disclosure may be defined by the isotopic enrichment factor. The term “isotopic enrichment factor” as used herein means the ratio between the isotopic abundance and the natural abundance of a specified isotope. In some embodiments, if a substituent in a compound of the disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### Combination Therapies

One aspect disclosed herein provides methods of treating CF and other CFTR-mediated diseases using Compound I, a deuterated derivative thereof, or a pharmaceutically acceptable salt of any of the foregoing, in combination with at least one additional active pharmaceutical ingredient (API).

Thus, in some embodiments, the invention provides methods of treating, lessening the severity of, or symptomatically treating cystic fibrosis in a patient comprising administering to the patient about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, alone or in combination with at least one additional active pharmaceutical ingredient, such as, e.g., a CFTR-modulating agent.

In some embodiments, the invention provides methods of treating, lessening the severity of, or symptomatically treating cystic fibrosis in a patient comprising administering to the patient about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, alone or in combination with at least one additional active pharmaceutical ingredient, such as, e.g., a CFTR-modulating agent.

In some embodiments, the invention provides methods of treating, lessening the severity of, or symptomatically treating cystic fibrosis in a patient comprising administering to the patient about 5 mg to about 375 mg (e.g., about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 5 mg to about 50 mg, about 5 mg to about 20 mg, about 5 mg to about 10 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, or about 375 mg) of Compound I, a deuterated derivative thereof, or a pharmaceutically acceptable salt of any of the foregoing, alone or in combination with at least one additional active pharmaceutical ingredient, such as, e.g., a CFTR-modulating agent.

In some embodiments, the invention provides methods of treating, lessening the severity of, or symptomatically treating cystic fibrosis in a patient comprising administering to the patient about 15 mg to about 350 mg (e.g., about 30 mg to about 300 mg, about 50 mg to about 200 mg, about 50 mg to about 175 mg, about 20 mg to about 100 mg, about 20 mg to about 30 mg, about 30 mg to about 60 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, or about 350 mg) of Compound I, a deuterated derivative thereof, or a pharmaceutically acceptable salt of any of the foregoing,, alone or in combination with at least one additional active pharmaceutical ingredient, such as, e.g., a CFTR-modulating agent.

In some embodiments, the invention provides methods of treating, lessening the severity of, or symptomatically treating cystic fibrosis in a patient comprising administering to the patient about 30 mg to about 300 mg (e.g., about 50 mg to about 200 mg, about 50 mg to about 175 mg, about 30 mg to about 60 mg, about 30 mg, about 31 mg, about 32 mg, about 33 mg, about 34 mg, about 35 mg, about 36 mg, about 37 mg, about 38 mg, about 39 mg, about 40 mg about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, or about 300 mg) of Compound I, a deuterated derivative thereof, or a pharmaceutically acceptable salt of any of the foregoing,, alone or in combination with at least one additional active pharmaceutical ingredient, such as, e.g., a CFTR-modulating agent.

In some embodiments, the invention provides methods of treating, lessening the severity of, or symptomatically treating cystic fibrosis in a patient comprising administering to the patient about 50 mg to about 200 mg (e.g., about 50 mg to about 175 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg) of Compound I, a deuterated derivative thereof, or a pharmaceutically acceptable salt of any of the foregoing, alone or in combination with at least one additional active pharmaceutical ingredient, such as, e.g., a CFTR-modulating agent.

In some embodiments, the invention provides methods of treating, lessening the severity of, or symptomatically treating cystic fibrosis in a patient comprising administering to the patient about 50 mg to about 175 mg (e.g., about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, or about 175 mg) of Compound I, a deuterated derivative thereof, or a pharmaceutically acceptable salt of any of the foregoing, alone or in combination with at least one additional active pharmaceutical ingredient, such as, e.g., a CFTR-modulating agent.

In some embodiments, the invention provides methods of treating, lessening the severity of, or symptomatically treating cystic fibrosis in a patient comprising administering to the patient about 10 mg to about 200 mg (e.g., about 50 mg to about 200 mg, about 50 mg to about 175 mg, about 20 mg to about 100 mg, about 20 mg to about 30 mg, about 30 mg to about 60 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg) of Compound I, a deuterated derivative thereof, or a pharmaceutically acceptable salt of any of the foregoing, alone or in combination with at least one additional active pharmaceutical ingredient, such as, e.g., a CFTR-modulating agent.

In some embodiments, the invention provides methods of treating, lessening the severity of, or symptomatically treating cystic fibrosis in a patient comprising administering to the patient about 20 mg to about 100 mg (e.g., about 20 mg to about 30 mg, about 30 mg to about 60 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, or about 100 mg) of Compound I, a deuterated derivative thereof, or a pharmaceutically acceptable salt of any of the foregoing, alone or in combination with at least one additional active pharmaceutical ingredient, such as, e.g., a CFTR-modulating agent.

In some embodiments, the invention provides methods of treating, lessening the severity of, or symptomatically treating cystic fibrosis in a patient comprising administering to the patient about 30 mg to about 60 mg (e.g., about 30 mg, about 31 mg, about 32 mg, about 33 mg, about 34 mg, about 35 mg, about 36 mg, about 37 mg, about 38 mg, about 39 mg, about 40 mg, or about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, or about 60 mg) of Compound I, a deuterated derivative thereof, or a pharmaceutically acceptable salt of any of the foregoing, alone or in combination with at least one additional active pharmaceutical ingredient, such as, e.g., a CFTR-modulating agent.

In some embodiments, the invention provides methods of treating, lessening the severity of, or symptomatically treating cystic fibrosis in a patient comprising administering to the patient about 1 mg to about 50 mg (e.g., about 2 mg to about 40 mg, about 5 mg to about 50 mg, about 20 mg to about 30 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, or about 50 mg) of Compound I, a deuterated derivative thereof, or a pharmaceutically acceptable salt of any of the foregoing, alone or in combination with at least one additional active pharmaceutical ingredient, such as, e.g., a CFTR-modulating agent.

In some embodiments, the invention provides methods of treating, lessening the severity of, or symptomatically treating cystic fibrosis in a patient comprising administering to the patient about 2 mg to about 40 mg (e.g., about 20 mg to about 30 mg, about 5 mg to about 20 mg, about 5 mg to about 10 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, or about 40 mg) of Compound I, a deuterated derivative thereof, or a pharmaceutically acceptable salt of any of the foregoing, alone or in combination with at least one additional active pharmaceutical ingredient, such as, e.g., a CFTR-modulating agent.

In some embodiments, the invention provides methods of treating, lessening the severity of, or symptomatically treating cystic fibrosis in a patient comprising administering to the patient about 5 mg to about 50 mg (e.g., about 20 mg to about 30 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, or about 50 mg) of Compound I, a deuterated derivative thereof, or a pharmaceutically acceptable salt of any of the foregoing, alone or in combination with at least one additional active pharmaceutical ingredient, such as, e.g., a CFTR-modulating agent.

In some embodiments, the invention provides methods of treating, lessening the severity of, or symptomatically treating cystic fibrosis in a patient comprising administering to the patient about 5 mg to about 20 mg (e.g., about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, or about 20 mg) of Compound I, a deuterated derivative thereof, or a pharmaceutically acceptable salt of any of the foregoing, alone or in combination with at least one additional active pharmaceutical ingredient, such as, e.g., a CFTR-modulating agent.

In some embodiments, the invention provides methods of treating, lessening the severity of, or symptomatically treating cystic fibrosis in a patient comprising administering to the patient about 5 mg to about 10 mg (e.g., about 5 mg, about 5.5 mg, about 6 mg, about 6.5 mg, about 7 mg, about 7.5 mg, about 8 mg, about 8.5 mg, about 9 mg, about 9.5 mg, or about 10 mg) of Compound I, a deuterated derivative thereof, or a pharmaceutically acceptable salt of any of the foregoing, alone or in combination with at least one additional active pharmaceutical ingredient, such as, e.g., a CFTR-modulating agent.

In some embodiments, the invention provides methods of treating, lessening the severity of, or symptomatically treating cystic fibrosis in a patient comprising administering to the patient about 1 mg to about 30 mg (e.g., about 5 mg to about 20 mg, about 20 mg to about 30 mg, about 5 mg to about 10 mg, about 1 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, or about 30 mg) of Compound I, a deuterated derivative thereof, or a pharmaceutically acceptable salt of any of the foregoing, alone or in combination with at least one additional active pharmaceutical ingredient, such as, e.g., a CFTR-modulating agent.

In some embodiments, at least one additional API is selected from mucolytic agents, bronchodilators, antibiotics, anti-infective agents, anti-inflammatory agents, and nutritional agents.

In some embodiments, the additional agent is a mucolyte. Exemplary mucolytes useful herein include Pulmozyme®.

In some embodiments, the additional agent is a bronchodilator. Exemplary bronchodilators include albuterol, metaprotenerol sulfate, pirbuterol acetate, salmeterol, and tetrabuline sulfate.

In some embodiments, the additional therapeutic agent is an antibiotic. Exemplary antibiotics useful herein include tobramycin, including tobramycin inhaled powder (TIP), azithromycin, aztreonam, including the aerosolized form of aztreonam, amikacin, including liposomal formulations thereof, ciprofloxacin, including formulations thereof suitable for administration by inhalation, levoflaxacin, including aerosolized formulations thereof, and combinations of two antibiotics, e.g., fosfomycin and tobramycin.

In some embodiments, the additional agent is an anti-inflammatory agent, i.e., an agent that can reduce the inflammation in the lungs. Exemplary such agents useful herein include ibuprofen, docosahexanoic acid (DHA), sildenafil, inhaled glutathione, pioglitazone, hydroxychloroquine, and simvastatin.

In some embodiments, the additional agent is a nutritional agent. Exemplary nutritional agents include pancrelipase (pancreating enzyme replacement), including PancreaseR, Pancreacarb®, Ultrase®, or Creon®, Liprotomase® (formerly Trizytek®), Aquadeks®, and glutathione inhalation. In one embodiment, the additional nutritional agent is pancrelipase.

In some embodiments, at least one additional active pharmaceutical ingredient is selected from CFTR-modulating agents. In some embodiments, the CFTR-modulating agent is a CFTR corrector. In some embodiments, the CFTR-modulating agent is a CFTR potentiator enhancer/co-potentiator (for example, ASP-11). In some embodiments, the at least one additional active pharmaceutical ingredient is a CFTR amplifier. In some embodiments, the at least one additional active pharmaceutical ingredient is a CFTR readthrough agent. In some embodiments, the at least one additional active pharmaceutical ingredient is a CFTR nucleic acid therapy.

In some embodiments, the at least one additional active pharmaceutical ingredient is an ENaC inhibitor. In some embodiments, the at least one additional active pharmaceutical ingredient is a TMEM16A modulator. In some embodiments, the at least one additional active pharmaceutical ingredient is a GPR39 agonist.

In some embodiments, the at least one additional active pharmaceutical ingredient is chosen from (a) Compound II, deuterated derivatives of Compound II and pharmaceutically acceptable salts of Compound II and deuterated derivatives thereof; (b) Compound III, deuterated derivatives of Compound III and pharmaceutically acceptable salts of Compound III and deuterated derivatives thereof; (c) Compound III-d, and pharmaceutically acceptable salts of Compound III-d; (d) Compound IV, deuterated derivatives of Compound IV and pharmaceutically acceptable salts of Compound IV and deuterated derivatives thereof; (e) Compound V, deuterated derivatives of Compound V and pharmaceutically acceptable salts of Compound V and deuterated derivatives thereof; (f) Compound VI, deuterated derivatives of Compound VI and pharmaceutically acceptable salts of Compound VI and deuterated derivatives thereof; (g) Compound VII, deuterated derivatives of Compound VII and pharmaceutically acceptable salts of Compound VII and deuterated derivatives thereof; (h) Compound VIII, deuterated derivatives of Compound VIII and pharmaceutically acceptable salts of Compound VIII and deuterated derivatives thereof; (i) Compound IX, deuterated derivatives of Compound IX and pharmaceutically acceptable salts of Compound IX and deuterated derivatives thereof; and (j) Compound X, deuterated derivatives of Compound X and pharmaceutically acceptable salts of Compound X and deuterated derivatives thereof. Thus, in some embodiments, the combination therapies provided herein comprise administering Compound I, a deuterated derivative thereof, or a pharmaceutically acceptable salt of any of the foregoing; and at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof.

In some embodiments, the combination therapies provided herein comprise administering (a) about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888.

In some embodiments, the combination therapies provided herein comprise administering (a) about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888.

In some embodiments, the combination therapies provided herein comprise administering (a) about 5 mg to about 375 mg (e.g., about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 5 mg to about 50 mg, about 5 mg to about 20 mg, about 5 mg to about 10 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, or about 375 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888.

In some embodiments, the combination therapies provided herein comprise administering (a) about 15 mg to about 350 mg (e.g., about 30 mg to about 300 mg, about 50 mg to about 200 mg, about 50 mg to about 175 mg, about 20 mg to about 100 mg, about 20 mg to about 30 mg, about 30 mg to about 60 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, or about 350 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; PCT International Application No. PCT/US2023/017627; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888.

In some embodiments, the combination therapies provided herein comprise administering (a) about 30 mg to about 300 mg (e.g., about 50 mg to about 200 mg, about 50 mg to about 175 mg, about 30 mg to about 60 mg, about 30 mg, about 31 mg, about 32 mg, about 33 mg, about 34 mg, about 35 mg, about 36 mg, about 37 mg, about 38 mg, about 39 mg, about 40 mg about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, or about 300 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; PCT International Application No. PCT/US2023/017627; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888.

In some embodiments, the combination therapies provided herein comprise administering (a) about 50 mg to about 200 mg (e.g., about 50 mg to about 175 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; PCT International Application No. PCT/US2023/017627; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888.

In some embodiments, the combination therapies provided herein comprise administering (a) about 50 mg to about 175 mg (e.g., about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, or about 175 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; PCT International Application No. PCT/US2023/017627; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888.

In some embodiments, the combination therapies provided herein comprise administering (a) about 10 mg to about 200 mg (e.g., about 50 mg to about 200 mg, about 50 mg to about 175 mg, about 20 mg to about 100 mg, about 20 mg to about 30 mg, about 30 mg to about 60 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; PCT International Application No. PCT/US2023/017627; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888.

In some embodiments, the combination therapies provided herein comprise administering (a) about 20 mg to about 100 mg (e.g., about 20 mg to about 30 mg, about 30 mg to about 60 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, or about 100 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; PCT International Application No. PCT/US2023/017627; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888.

In some embodiments, the combination therapies provided herein comprise administering (a) about 30 mg to about 60 mg (e.g., about 30 mg, about 31 mg, about 32 mg, about 33 mg, about 34 mg, about 35 mg, about 36 mg, about 37 mg, about 38 mg, about 39 mg, about 40 mg, or about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, or about 60 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; PCT International Application No. PCT/US2023/017627; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888.

In some embodiments, the combination therapies provided herein comprise administering (a) about 1 mg to about 50 mg (e.g., about 2 mg to about 40 mg, about 5 mg to about 50 mg, about 5 mg to about 20 mg, about 20 mg to about 30 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, or about 50 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; PCT International Application No. PCT/US2023/017627; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888.

In some embodiments, the combination therapies provided herein comprise administering (a) about 2 mg to about 40 mg (e.g., about 5 mg to about 20 mg, about 20 mg to about 30 mg, about 5 mg to about 10 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, or about 40 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; PCT International Application No. PCT/US2023/017627; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888.

In some embodiments, the combination therapies provided herein comprise administering (a) about 5 mg to about 50 mg (e.g., about 20 mg to about 30 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, or about 50 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; PCT International Application No. PCT/US2023/017627; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888.

In some embodiments, the combination therapies provided herein comprise administering (a) about 5 mg to about 20 mg (e.g., about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, or about 20 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; PCT International Application No. PCT/US2023/017627; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888.

In some embodiments, the combination therapies provided herein comprise administering (a) about 5 mg to about 10 mg (e.g., about 5 mg, about 5.5 mg, about 6 mg, about 6.5 mg, about 7 mg, about 7.5 mg, about 8 mg, about 8.5 mg, about 9 mg, about 9.5 mg, or about 10 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; PCT International Application No. PCT/US2023/017627; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888.

In some embodiments, the combination therapies provided herein comprise administering (a) about 1 mg to about 30 mg (e.g., about 5 mg to about 20 mg, about 20 mg to about 30 mg, about 5 mg to about 10 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, or about 30 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; PCT International Application No. PCT/US2023/017627; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888.

In some embodiments, the combination therapies provided herein comprise administering (a) about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor), GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039.

In some embodiments, the combination therapies provided herein comprise administering (a) about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor), GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039.

In some embodiments, the combination therapies provided herein comprise administering (a) about 5 mg to about 375 mg (e.g., about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 5 mg to about 50 mg, about 5 mg to about 20 mg, about 5 mg to about 10 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, or about 375 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor), GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039.

In some embodiments, the combination therapies provided herein comprise administering (a) about 15 mg to about 350 mg (e.g., about 30 mg to about 300 mg, about 50 mg to about 200 mg, about 50 mg to about 175 mg, about 20 mg to about 100 mg, about 20 mg to about 30 mg, about 30 mg to about 60 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, or about 350 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor),
GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039.

In some embodiments, the combination therapies provided herein comprise administering (a) about 30 mg to about 300 mg (e.g., about 50 mg to about 200 mg, about 50 mg to about 175 mg, about 30 mg to about 60 mg, about 30 mg, about 31 mg, about 32 mg, about 33 mg, about 34 mg, about 35 mg, about 36 mg, about 37 mg, about 38 mg, about 39 mg, about 40 mg about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, or about 300 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor), GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039.

In some embodiments, the combination therapies provided herein comprise administering (a) about 50 mg to about 200 mg (e.g., about 50 mg to about 175 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor), GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039.

In some embodiments, the combination therapies provided herein comprise administering (a) about 50 mg to about 175 mg (e.g., about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, or about 175 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII,
Compound IX, Compound X, and deuterated derivatives and pharmaceutically acceptable salts thereof; and (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor), GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039.

In some embodiments, the combination therapies provided herein comprise administering (a) about 10 mg to about 200 mg (e.g., about 50 mg to about 200 mg, about 50 mg to about 175 mg, about 20 mg to about 100 mg, about 20 mg to about 30 mg, about 30 mg to about 60 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor), GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039.

In some embodiments, the combination therapies provided herein comprise administering (a) about 20 mg to about 100 mg (e.g., about 20 mg to about 30 mg, about 30 mg to about 60 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, or about 100 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974,
GLPG2451/ABBV-2451, QBW251 (Icenticaftor), GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039.

In some embodiments, the combination therapies provided herein comprise administering (a) about 30 mg to about 60 mg (e.g., about 30 mg, about 31 mg, about 32 mg, about 33 mg, about 34 mg, about 35 mg, about 36 mg, about 37 mg, about 38 mg, about 39 mg, about 40 mg, or about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, or about 60 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor), GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039.

In some embodiments, the combination therapies provided herein comprise administering (a) about 1 mg to about 50 mg (e.g., about 2 mg to about 40 mg, about 5 mg to about 50 mg, about 5 mg to about 20 mg, about 20 mg to about 30 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, or about 50 mg) of Compound I, a deuterated derivative thereof, or a pharmaceutically acceptable salt of any of the foregoing;
(b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor), GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039.

In some embodiments, the combination therapies provided herein comprise administering (a) about 2 mg to about 40 mg (e.g., about 5 mg to about 20 mg, about 5 mg to about 10 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, or about 40 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor), GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039.

In some embodiments, the combination therapies provided herein comprise administering (a) about 5 mg to about 50 mg (e.g., about 20 mg to about 30 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, or about 50 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII,
Compound IX, Compound X, and deuterated derivatives and pharmaceutically acceptable salts thereof; and (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor), GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039.

In some embodiments, the combination therapies provided herein comprise administering (a) about 5 mg to about 20 mg (e.g., about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, or about 20 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor),
GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039.

In some embodiments, the combination therapies provided herein comprise administering (a) about 5 mg to about 10 mg (e.g., about 5 mg, about 5.5 mg, about 6 mg, about 6.5 mg, about 7 mg, about 7.5 mg, about 8 mg, about 8.5 mg, about 9 mg, about 9.5 mg, or about 10 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor), GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039.

In some embodiments, the combination therapies provided herein comprise administering (a) about 1 mg to about 30 mg (e.g., about 5 mg to about 20 mg, about 20 mg to about 30 mg, about 5 mg to about 10 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, or about 30 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; and (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor), GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039.

In some embodiments, about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, is administered in combination with at least one compound chosen from Compound II and pharmaceutically acceptable salts and deuterated derivatives thereof.

In some embodiments, about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, is administered in combination with at least one compound chosen from Compound III and pharmaceutically acceptable salts and deuterated derivatives thereof.

In some embodiments, about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, is administered in combination with at least one compound chosen from Compound III-d and pharmaceutically acceptable salts thereof.

In some embodiments, about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, is administered in combination with at least one compound chosen from Compound IV and deuterated derivatives and pharmaceutically acceptable salts thereof.

In some embodiments, about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, is administered in combination with at least one compound chosen from Compound V and deuterated derivatives and pharmaceutically acceptable salts thereof.

In some embodiments, about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, is administered in combination with at least one compound chosen from Compound VI and deuterated derivatives and pharmaceutically acceptable salts thereof.

In some embodiments, about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, is administered in combination with at least one compound chosen from Compound VII and deuterated derivatives and pharmaceutically acceptable salts thereof.

In some embodiments, about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, is administered in combination with at least one compound chosen from Compound VIII and deuterated derivatives and pharmaceutically acceptable salts thereof.

In some embodiments, about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, is administered in combination with at least one compound chosen from Compound IX and deuterated derivatives and pharmaceutically acceptable salts thereof.

In some embodiments, about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, is administered in combination with at least one compound chosen from Compound X and deuterated derivatives and pharmaceutically acceptable salts thereof.

In some embodiments, about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, is administered in combination with at least one compound chosen from Compound II and pharmaceutically acceptable salts and deuterated derivatives thereof.

In some embodiments, about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, is administered in combination with at least one compound chosen from Compound III and pharmaceutically acceptable salts and deuterated derivatives thereof.

In some embodiments, about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, is administered in combination with at least one compound chosen from Compound III-d and pharmaceutically acceptable salts thereof.

In some embodiments, about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, is administered in combination with at least one compound chosen from Compound IV and deuterated derivatives and pharmaceutically acceptable salts thereof.

In some embodiments, about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, is administered in combination with at least one compound chosen from Compound V and deuterated derivatives and pharmaceutically acceptable salts thereof.

In some embodiments, about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, is administered in combination with at least one compound chosen from Compound VI and deuterated derivatives and pharmaceutically acceptable salts thereof.

In some embodiments, about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, is administered in combination with at least one compound chosen from Compound VII and deuterated derivatives and pharmaceutically acceptable salts thereof.

In some embodiments, about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, is administered in combination with at least one compound chosen from Compound VIII and deuterated derivatives and pharmaceutically acceptable salts thereof.

In some embodiments, about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, is administered in combination with at least one compound chosen from Compound IX and deuterated derivatives and pharmaceutically acceptable salts thereof.

In some embodiments, about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, is administered in combination with at least one compound chosen from Compound X and deuterated derivatives and pharmaceutically acceptable salts thereof.

In some embodiments, about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, is administered in combination with at least one compound chosen from Compound II and deuterated derivatives and pharmaceutically acceptable salts thereof, and at least one compound chosen from Compound V and pharmaceutically acceptable salts and deuterated derivatives thereof.

In some embodiments, about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, is administered in combination with at least one compound chosen from Compound II and deuterated derivatives and pharmaceutically acceptable salts thereof, and at least one compound chosen from Compound VI and deuterated derivatives and pharmaceutically acceptable salts thereof.

In some embodiments, about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, is administered in combination with at least one compound chosen from Compound II and deuterated derivatives and pharmaceutically acceptable salts thereof, and at least one compound chosen from Compound VII and deuterated derivatives and pharmaceutically acceptable salts thereof.

In some embodiments, about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, is administered in combination with at least one compound chosen from Compound II and deuterated derivatives and pharmaceutically acceptable salts thereof, and at least one compound chosen from Compound VIII and deuterated derivatives and pharmaceutically acceptable salts thereof.

In some embodiments, about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, is administered in combination with at least one compound chosen from Compound II and deuterated derivatives and pharmaceutically acceptable salts thereof, and at least one compound chosen from Compound V and pharmaceutically acceptable salts and deuterated derivatives thereof.

In some embodiments, about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, is administered in combination with at least one compound chosen from Compound II and deuterated derivatives and pharmaceutically acceptable salts thereof, and at least one compound chosen from Compound VI and deuterated derivatives and pharmaceutically acceptable salts thereof.

In some embodiments, about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, is administered in combination with at least one compound chosen from Compound II and deuterated derivatives and pharmaceutically acceptable salts thereof, and at least one compound chosen from Compound VII and deuterated derivatives and pharmaceutically acceptable salts thereof.

In some embodiments, about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, is administered in combination with at least one compound chosen from Compound II and deuterated derivatives and pharmaceutically acceptable salts thereof, and at least one compound chosen from Compound VIII and deuterated derivatives and pharmaceutically acceptable salts thereof.

Each of Compound I, a deuterated derivative thereof, or a pharmaceutically acceptable salt of any of the foregoing, and compounds selected from Compound II, Compound III, Compound III-d, Compound IV, Compound V, Compound VI, Compound VII, Compound VIII, Compound IX, Compound X, and deuterated derivatives and pharmaceutically acceptable salts thereof, independently can be administered once daily, twice daily, or three times daily.

In some embodiments, Compound I, a deuterated derivative thereof, or a pharmaceutically acceptable salt of any of the foregoing, is administered once daily. In some embodiments, Compound I, a deuterated derivative thereof, or a pharmaceutically acceptable salt of any of the foregoing, is administered twice daily. In some embodiments, Compound I, a deuterated derivative thereof, or a pharmaceutically acceptable salt of any of the foregoing, and at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof are administered once daily. In some embodiments, Compound I, a deuterated derivative thereof, or a pharmaceutically acceptable salt of any of the foregoing, and at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof are administered twice daily. In some embodiments, Compound I, a deuterated derivative thereof, or a pharmaceutically acceptable salt of any of the foregoing, and at least one compound chosen from Compound II and deuterated derivatives and pharmaceutically acceptable salts thereof are administered once daily. In some embodiments Compound I, a deuterated derivative thereof, or a pharmaceutically acceptable salt of any of the foregoing, and at least one compound chosen from Compound II and deuterated derivatives and pharmaceutically acceptable salts thereof are administered twice daily.

Compound I, a deuterated derivative thereof, or a pharmaceutically acceptable salt of any of the foregoing, and an additional therapeutic agent, can be administered in a single pharmaceutical composition or separate pharmaceutical compositions. Thus, (a) about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, and (b) a compound selected from Compound II, Compound III, Compound III-d, Compound IV, Compound V, Compound VI, Compound VII, Compound VIII, Compound IX, Compound X, and deuterated derivatives and pharmaceutically acceptable salts thereof, can be administered in a single pharmaceutical composition comprising (a) and (b), or in separate pharmaceutical compositions wherein the first composition comprises (a) and the second composition comprises (b). Such pharmaceutical compositions can be administered once daily or multiple times daily, such as two or three times daily. As used herein, the phrase that a given amount of API (e.g., Compound I, a deuterated derivative thereof, or a pharmaceutically acceptable salt of any of the foregoing) is administered once or twice daily or per day means that said given amount is administered per dosing, which may occur once or twice daily.

In some embodiments, about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, is administered in a first pharmaceutical composition; and at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof is administered in a second pharmaceutical composition.

Compound I, a deuterated derivative thereof, or a pharmaceutically acceptable salt of any of the foregoing, and an additional therapeutic agent, can be administered in a single pharmaceutical composition or separate pharmaceutical compositions. Thus, (a) about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, and (b) a compound selected from Compound II, Compound III, Compound III-d, Compound IV, Compound V, Compound VI, Compound VII, Compound VIII, Compound IX, Compound X, and deuterated derivatives and pharmaceutically acceptable salts thereof, can be administered in a single pharmaceutical composition comprising (a) and (b), or in separate pharmaceutical compositions wherein the first composition comprises (a) and the second composition comprises (b). Such pharmaceutical compositions can be administered once daily or multiple times daily, such as two or three times daily. As used herein, the phrase that a given amount of API (e.g., Compound I, a deuterated derivative thereof, or a pharmaceutically acceptable salt of any of the foregoing) is administered once or twice daily or per day means that said given amount is administered per dosing, which may occur once or twice daily.

In some embodiments, about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, is administered in a first pharmaceutical composition; and at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof is administered in a second pharmaceutical composition.

Any suitable pharmaceutical compositions known in the art can be used for Compound I, Compound II, Compound III, Compound III-d, Compound IV, Compound V, Compound VI, Compound VII, Compound VIII, Compound IX, Compound X, and deuterated derivatives and pharmaceutically acceptable salts thereof. Some exemplary pharmaceutical compositions for Compound II and its pharmaceutically acceptable salts can be found in WO 2011/119984 and WO 2014/014841, incorporated herein by reference. Some exemplary pharmaceutical compositions for Compound III and its pharmaceutically acceptable salts can be found in WO 2007/134279, WO 2010/019239, WO 2011/019413, WO 2012/027731, and WO 2013/130669, and some exemplary pharmaceutical compositions for Compound III-d and its pharmaceutically acceptable salts can be found in U.S. Pat. Nos. 8,865,902, 9,181,192, 9,512,079, WO 2017/053455, and WO 2018/080591, all of which are incorporated herein by reference. Some exemplary pharmaceutical compositions for Compound IV and its pharmaceutically acceptable salts can be found in WO 2010/037066, WO 2011/127421, and WO 2014/071122, incorporated herein by reference. Some exemplary pharmaceutical compositions for Compound V and its pharmaceutically acceptable salts can be found in WO 2019/152940, incorporated herein by reference. Some exemplary pharmaceutical compositions for Compound VI and its pharmaceutically acceptable salts can be found in WO 2019/079760, incorporated herein by reference.

### Pharmaceutical Compositions

Another aspect of the invention provides a pharmaceutical composition comprising Compound I, a deuterated derivative thereof, or a pharmaceutically acceptable salt of any of the foregoing, and at least one pharmaceutically acceptable carrier.

In some embodiments, the disclosure provides a pharmaceutical composition comprising (a) about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, and (b) at least one pharmaceutically acceptable carrier.

In some embodiments, the disclosure provides a pharmaceutical composition comprising (a) about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, and (b) at least one pharmaceutically acceptable carrier.

In some embodiments, the disclosure provides a pharmaceutical composition comprising (a) about 5 mg to about 375 mg (e.g., about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 5 mg to about 50 mg, about 5 mg to about 20 mg, about 5 mg to about 10 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, or about 375 mg) of Compound I, a deuterated derivative thereof, or a pharmaceutically acceptable salt of any of the foregoing, and (b) at least one pharmaceutically acceptable carrier.

In some embodiments, the disclosure provides a pharmaceutical composition comprising (a) about 15 mg to about 350 mg (e.g., about 30 mg to about 300 mg, about 50 mg to about 200 mg, about 50 mg to about 175 mg, about 20 mg to about 100 mg, about 20 mg to about 30 mg, about 30 mg to about 60 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, or about 350 mg) of Compound I, a deuterated derivative thereof, or a pharmaceutically acceptable salt of any of the foregoing, and (b) at least one pharmaceutically acceptable carrier.

In some embodiments, the disclosure provides a pharmaceutical composition comprising (a) about 30 mg to about 300 mg (e.g., about 50 mg to about 200 mg, about 50 mg to about 175 mg, about 30 mg to about 60 mg, about 30 mg, about 31 mg, about 32 mg, about 33 mg, about 34 mg, about 35 mg, about 36 mg, about 37 mg, about 38 mg, about 39 mg, about 40 mg about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, or about 300 mg) of Compound I, a deuterated derivative thereof, or a pharmaceutically acceptable salt of any of the foregoing, and (b) at least one pharmaceutically acceptable carrier.

In some embodiments, the disclosure provides a pharmaceutical composition comprising (a) about 50 mg to about 200 mg (e.g., about 50 mg to about 175 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg) of Compound I, a deuterated derivative thereof, or a pharmaceutically acceptable salt of any of the foregoing, and (b) at least one pharmaceutically acceptable carrier.

In some embodiments, the disclosure provides a pharmaceutical composition comprising (a) about 50 mg to about 175 mg (e.g., about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, or about 175 mg) of Compound I, a deuterated derivative thereof, or a pharmaceutically acceptable salt of any of the foregoing, and (b) at least one pharmaceutically acceptable carrier.

In some embodiments, the disclosure provides a pharmaceutical composition comprising (a) about 10 mg to about 200 mg (e.g., about 50 mg to about 200 mg, about 50 mg to about 175 mg, about 20 mg to about 100 mg, about 20 mg to about 30 mg, about 30 mg to about 60 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg) of Compound I, a deuterated derivative thereof, or a pharmaceutically acceptable salt of any of the foregoing, and (b) at least one pharmaceutically acceptable carrier.

In some embodiments, the disclosure provides a pharmaceutical composition comprising (a) about 20 mg to about 100 mg (e.g., about 30 mg to about 60 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, or about 100 mg) of Compound I, a deuterated derivative thereof, or a pharmaceutically acceptable salt of any of the foregoing, and (b) at least one pharmaceutically acceptable carrier.

In some embodiments, the disclosure provides a pharmaceutical composition comprising (a) about 30 mg to about 60 mg (e.g., about 30 mg, about 31 mg, about 32 mg, about 33 mg, about 34 mg, about 35 mg, about 36 mg, about 37 mg, about 38 mg, about 39 mg, about 40 mg, or about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, or about 60 mg) of Compound I, a deuterated derivative thereof, or a pharmaceutically acceptable salt of any of the foregoing, and (b) at least one pharmaceutically acceptable carrier. In some embodiments, the disclosure provides a pharmaceutical composition

comprising (a) about 1 mg to about 50 mg (e.g., about 2 mg to about 40 mg, about 5 mg to about 50 mg, about 5 mg to about 20 mg, about 20 mg to about 30 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, or about 50 mg) of Compound I, a deuterated derivative thereof, or a pharmaceutically acceptable salt of any of the foregoing, and (b) at least one pharmaceutically acceptable carrier.

In some embodiments, the disclosure provides a pharmaceutical composition comprising (a) about 2 mg to about 40 mg (e.g., about 5 mg to about 20 mg, about 20 mg to about 30 mg, about 5 mg to about 10 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, or about 40 mg) of Compound I, a deuterated derivative thereof, or a pharmaceutically acceptable salt of any of the foregoing, and (b) at least one pharmaceutically acceptable carrier.

In some embodiments, the disclosure provides a pharmaceutical composition comprising (a) about 5 mg to about 50 mg (e.g., about 20 mg to about 30 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, or about 50 mg) of Compound I, a deuterated derivative thereof, or a pharmaceutically acceptable salt of any of the foregoing, and (b) at least one pharmaceutically acceptable carrier.

In some embodiments, the disclosure provides a pharmaceutical composition comprising (a) about 5 mg to about 20 mg (e.g., about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, or about 20 mg) of Compound I, a deuterated derivative thereof, or a pharmaceutically acceptable salt of any of the foregoing, and (b) at least one pharmaceutically acceptable carrier.

In some embodiments, the disclosure provides a pharmaceutical composition comprising (a) about 5 mg to about 10 mg (e.g., about 5 mg, about 5.5 mg, about 6 mg, about 6.5 mg, about 7 mg, about 7.5 mg, about 8 mg, about 8.5 mg, about 9 mg, about 9.5 mg, or about 10 mg) of Compound I, a deuterated derivative thereof, or a pharmaceutically acceptable salt of any of the foregoing, and (b) at least one pharmaceutically acceptable carrier.

In some embodiments, the disclosure provides a pharmaceutical composition comprising (a) about 1 mg to about 30 mg (e.g., about 5 mg to about 20 mg, about 20 mg to about 30 mg, about 5 mg to about 10 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, or about 30 mg) of Compound I, a deuterated derivative thereof, or a pharmaceutically acceptable salt of any of the foregoing, and (b) at least one pharmaceutically acceptable carrier.

In some embodiments, the invention provides pharmaceutical compositions comprising about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, and optionally at least one additional active pharmaceutical ingredient. In some embodiments, the at least one additional active pharmaceutical ingredient is a CFTR modulator. In some embodiments, the at least one additional active pharmaceutical ingredient is a CFTR corrector. In some embodiments, the at least one additional active pharmaceutical ingredient is a CFTR potentiator. In some embodiments, the at least one additional active pharmaceutical ingredient is a compound that enhances CFTR potentiation, i.e., a CFTR potentiator enhancer/co-potentiator. In some embodiments, the at least one additional active pharmaceutical ingredient is a CFTR amplifier. In some embodiments, the at least one additional active pharmaceutical ingredient is a CFTR readthrough agent. In some embodiments, the at least one additional active pharmaceutical ingredient is a CFTR nucleic acid therapy. In some embodiments, the at least one additional active pharmaceutical ingredient is an ENaC inhibitor. In some embodiments, the at least one additional active pharmaceutical ingredient is a TMEM16A modulator. In some embodiments, the at least one additional active pharmaceutical ingredient is a GPR39 agonist. In some embodiments, the pharmaceutical composition comprises Compound I, a deuterated derivative thereof, or a pharmaceutically acceptable salt of any of the foregoing, and at least one additional active pharmaceutical ingredient, which is a CFTR corrector. In some embodiments, the pharmaceutical composition comprises Compound I, a deuterated derivative thereof, or a pharmaceutically acceptable salt of any of the foregoing, and at least two additional active pharmaceutical ingredients, each of which is a CFTR corrector.

In some embodiments, the invention provides pharmaceutical compositions comprising about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, and optionally at least one additional active pharmaceutical ingredient. In some embodiments, the at least one additional active pharmaceutical ingredient is a CFTR modulator. In some embodiments, the at least one additional active pharmaceutical ingredient is a CFTR corrector. In some embodiments, the at least one additional active pharmaceutical ingredient is a CFTR potentiator. In some embodiments, the at least one additional active pharmaceutical ingredient is a compound that enhances CFTR potentiation, i.e., a CFTR potentiator enhancer/co-potentiator. In some embodiments, the at least one additional active pharmaceutical ingredient is a CFTR amplifier. In some embodiments, the at least one additional active pharmaceutical ingredient is a CFTR readthrough agent. In some embodiments, the at least one additional active pharmaceutical ingredient is a CFTR nucleic acid therapy. In some embodiments, the at least one additional active pharmaceutical ingredient is an ENaC inhibitor. In some embodiments, the at least one additional active pharmaceutical ingredient is a TMEM16A modulator. In some embodiments, the at least one additional active pharmaceutical ingredient is a GPR39 agonist. In some embodiments, the pharmaceutical composition comprises Compound I, a deuterated derivative thereof, or a pharmaceutically acceptable salt of any of the foregoing, and at least one additional active pharmaceutical ingredient, which is a CFTR corrector. In some embodiments, the pharmaceutical composition comprises Compound I, a deuterated derivative thereof, or a pharmaceutically acceptable salt of any of the foregoing, and at least two additional active pharmaceutical ingredients, each of which is a CFTR corrector.

In some embodiments, the invention provides a pharmaceutical composition comprising (a) about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, (b) at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof, and (c) at least one pharmaceutically acceptable carrier.

In some embodiments, the invention provides a pharmaceutical composition comprising (a) about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, (b) at least two compounds chosen from Compound II, Compound III, Compound III-d, Compound IV, Compound V, Compound VI, Compound VII, Compound VIII, Compound IX, Compound X, and deuterated derivatives and pharmaceutically acceptable salts thereof, and (c) at least one pharmaceutically acceptable carrier.

In some embodiments, the invention provides a pharmaceutical composition comprising (a) about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, (b) at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof, and (c) at least one pharmaceutically acceptable carrier.

In some embodiments, the invention provides a pharmaceutical composition comprising (a) about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, (b) at least two compounds chosen from Compound II, Compound III, Compound III-d, Compound IV, Compound V, Compound VI, Compound VII, Compound VIII, Compound IX, Compound X, and deuterated derivatives and pharmaceutically acceptable salts thereof, and (c) at least one pharmaceutically acceptable carrier.

In some embodiments, the invention provides a pharmaceutical composition comprising (a) about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, (b) at least one compound chosen from Compound II and deuterated derivatives and pharmaceutically acceptable salts thereof, and (c) at least one pharmaceutically acceptable carrier.

In some embodiments, the invention provides a pharmaceutical composition comprising (a) about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, (b) at least one compound chosen from Compound II and deuterated derivatives and pharmaceutically acceptable salts thereof, and (c) at least one pharmaceutically acceptable carrier.

In some embodiments, the disclosure provides a pharmaceutical composition comprising (a) about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, (b) at least one compound chosen from Compound II, and deuterated derivatives and pharmaceutically acceptable salts thereof, (c) at least one compound chosen from Compound III, Compound III-d, Compound IV, Compound V, Compound VI, Compound VII, Compound VIII, Compound IX, Compound X, and deuterated derivatives and pharmaceutically acceptable salts thereof, and (d) at least one pharmaceutically acceptable carrier.

In some embodiments, the disclosure provides a pharmaceutical composition comprising (a) about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, (b) at least one compound chosen from Compound II, and deuterated derivatives and pharmaceutically acceptable salts thereof, (c) at least one compound chosen from Compound III, Compound III-d, Compound IV, Compound V, Compound VI, Compound VII, Compound VIII, Compound IX, Compound X, and deuterated derivatives and pharmaceutically acceptable salts thereof, and (d) at least one pharmaceutically acceptable carrier.

In some embodiments, the pharmaceutical compositions provided herein comprise (a) about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; PCT International Application No. PCT/US2023/017627; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888; and (d) at least one pharmaceutically acceptable carrier.

In some embodiments, the pharmaceutical compositions provided herein comprise (a) about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; PCT International Application No. PCT/US2023/017627; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888; and (d) at least one pharmaceutically acceptable carrier.

In some embodiments, the pharmaceutical compositions provided herein comprise (a) about 5 mg to about 375 mg (e.g., about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 5 mg to about 50 mg, about 5 mg to about 20 mg, about 5 mg to about 10 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, or about 375 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; PCT International Application No. PCT/US2023/017627; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888; and (d) at least one pharmaceutically acceptable carrier.

In some embodiments, the pharmaceutical compositions provided herein comprise (a) about 15 mg to about 350 mg (e.g., about 30 mg to about 300 mg, about 50 mg to about 200 mg, about 50 mg to about 175 mg, about 20 mg to about 100 mg, about 20 mg to about 30 mg, about 30 mg to about 60 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, or about 350 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888; and (d) at least one pharmaceutically acceptable carrier.

In some embodiments, the pharmaceutical compositions provided herein comprise (a) about 30 mg to about 300 mg (e.g., about 50 mg to about 200 mg, about 50 mg to about 175 mg, about 30 mg to about 60 mg, about 30 mg, about 31 mg, about 32 mg, about 33 mg, about 34 mg, about 35 mg, about 36 mg, about 37 mg, about 38 mg, about 39 mg, about 40 mg about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, or about 300 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; PCT International Application No. PCT/US2023/017627; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888; and (d) at least one pharmaceutically acceptable carrier.

In some embodiments, the pharmaceutical compositions provided herein comprise (a) about 50 mg to about 200 mg (e.g., about 50 mg to about 175 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; PCT International Application No. PCT/US2023/017627; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888; and (d) at least one pharmaceutically acceptable carrier.

In some embodiments, the pharmaceutical compositions provided herein comprise (a) about 50 mg to about 175 mg (e.g., about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, or about 175 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; PCT International Application No. PCT/US2023/017627; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888; and (d) at least one pharmaceutically acceptable carrier.

In some embodiments, the pharmaceutical compositions provided herein comprise (a) about 10 mg to about 200 mg (e.g., about 50 mg to about 200 mg, about 50 mg to about 175 mg, about 20 mg to about 100 mg, about 20 mg to about 30 mg, about 30 mg to about 60 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; PCT International Application No. PCT/US2023/017627; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888; and (d) at least one pharmaceutically acceptable carrier.

In some embodiments, the pharmaceutical compositions provided herein comprise (a) about 20 mg to about 100 mg (e.g., about 20 mg to about 30 mg, about 30 mg to about 60 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, or about 100 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; PCT International Application No. PCT/US2023/017627; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888; and (d) at least one pharmaceutically acceptable carrier.

In some embodiments, the pharmaceutical compositions provided herein comprise (a) about 30 mg to about 60 mg (e.g., about 30 mg, about 31 mg, about 32 mg, about 33 mg, about 34 mg, about 35 mg, about 36 mg, about 37 mg, about 38 mg, about 39 mg, about 40 mg, or about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, or about 60 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; PCT International Application No. PCT/US2023/017627; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888; and (d) at least one pharmaceutically acceptable carrier.

In some embodiments, the pharmaceutical compositions provided herein comprise (a) about 1 mg to about 50 mg (e.g., about 2 mg to about 40 mg, about 5 mg to about 50 mg, about 5 mg to about 20 mg, about 20 mg to about 30 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, or about 50 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; PCT International Application No. PCT/US2023/017627; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888; and (d) at least one pharmaceutically acceptable carrier.

In some embodiments, the pharmaceutical compositions provided herein comprise (a) about 2 mg to about 40 mg (e.g., about 5 mg to about 20 mg, about 20 mg to about 30 mg, about 5 mg to about 10 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, or about 40 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; PCT International Application No. PCT/US2023/017627; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888; and (d) at least one pharmaceutically acceptable carrier.

In some embodiments, the pharmaceutical compositions provided herein comprise (a) about 5 mg to about 50 mg (e.g., about 20 mg to about 30 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, or about 50 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; PCT International Application No. PCT/US2023/017627; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888; and (d) at least one pharmaceutically acceptable carrier.

In some embodiments, the pharmaceutical compositions provided herein comprise (a) about 5 mg to about 20 mg (e.g., about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, or about 20 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; PCT International Application No. PCT/US2023/017627; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888; and (d) at least one pharmaceutically acceptable carrier.

In some embodiments, the pharmaceutical compositions provided herein comprise (a) about 5 mg to about 10 mg (e.g., about 5 mg, about 5.5 mg, about 6 mg, about 6.5 mg, about 7 mg, about 7.5 mg, about 8 mg, about 8.5 mg, about 9 mg, about 9.5 mg, or about 10 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; PCT International Application No. PCT/US2023/017627; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888; and (d) at least one pharmaceutically acceptable carrier.

In some embodiments, the pharmaceutical compositions provided herein comprise (a) about 1 mg to about 30 mg (e.g., about 5 mg to about 20 mg, about 20 mg to about 30 mg, about 5 mg to about 10 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, or about 30 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; PCT International Application No. PCT/US2023/017627; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888; and (d) at least one pharmaceutically acceptable carrier.

In some embodiments, the pharmaceutical compositions provided herein comprise (a) about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor), GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039; and (d) at least one pharmaceutically acceptable carrier.

In some embodiments, the pharmaceutical compositions provided herein comprise (a) about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor), GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039; and (d) at least one pharmaceutically acceptable carrier.

In some embodiments, the pharmaceutical compositions provided herein comprise (a) about 5 mg to about 375 mg (e.g., about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 5 mg to about 50 mg, about 5 mg to about 20 mg, about 5 mg to about 10 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, or about 375 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor),
GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039; and (d) at least one pharmaceutically acceptable carrier.

In some embodiments, the pharmaceutical compositions provided herein comprise (a) about 15 mg to about 350 mg (e.g., about 30 mg to about 300 mg, about 50 mg to about 200 mg, about 50 mg to about 175 mg, about 20 mg to about 100 mg, about 20 mg to about 30 mg, about 30 mg to about 60 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, or about 350 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor), GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039; and (d) at least one pharmaceutically acceptable carrier.

In some embodiments, the pharmaceutical compositions provided herein comprise (a) about 30 mg to about 300 mg (e.g., about 50 mg to about 200 mg, about 50 mg to about 175 mg, about 30 mg to about 60 mg, about 30 mg, about 31 mg, about 32 mg, about 33 mg, about 34 mg, about 35 mg, about 36 mg, about 37 mg, about 38 mg, about 39 mg, about 40 mg about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, or about 300 mg) of Compound I, a deuterated derivative thereof, or a pharmaceutically acceptable salt of any of the foregoing;
(b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor),
GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039; and (d) at least one pharmaceutically acceptable carrier.

In some embodiments, the pharmaceutical compositions provided herein comprise (a) about 50 mg to about 200 mg (e.g., about 50 mg to about 175 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII,
Compound IX, Compound X, and deuterated derivatives and pharmaceutically acceptable salts thereof; (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor),
GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039; and (d) at least one pharmaceutically acceptable carrier.

In some embodiments, the pharmaceutical compositions provided herein comprise (a) about 50 mg to about 175 mg (e.g., about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, or about 175 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor), GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039; and (d) at least one pharmaceutically acceptable carrier.

In some embodiments, the pharmaceutical compositions provided herein comprise (a) about 10 mg to about 200 mg (e.g., about 50 mg to about 200 mg, about 50 mg to about 175 mg, about 20 mg to about 100 mg, about 20 mg to about 30 mg, about 30 mg to about 60 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor), GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039; and (d) at least one pharmaceutically acceptable carrier.

In some embodiments, the pharmaceutical compositions provided herein comprise (a) about 20 mg to about 100 mg (e.g., about 20 mg to about 30 mg, about 30 mg to about 60 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, or about 100 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor),
GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039; and (d) at least one pharmaceutically acceptable carrier.

In some embodiments, the pharmaceutical compositions provided herein comprise (a) about 30 mg to about 60 mg (e.g., about 30 mg, about 31 mg, about 32 mg, about 33 mg, about 34 mg, about 35 mg, about 36 mg, about 37 mg, about 38 mg, about 39 mg, about 40 mg, or about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, or about 60 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor), GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039; and (d) at least one pharmaceutically acceptable carrier.

In some embodiments, the pharmaceutical compositions provided herein comprise (a) about 1 mg to about 50 mg (e.g., about 2 mg to about 40 mg, about 5 mg to about 50 mg, about 5 mg to about 20 mg, about 20 mg to about 30 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, or about 50 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor), GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039; and (d) at least one pharmaceutically acceptable carrier.

In some embodiments, the pharmaceutical compositions provided herein comprise (a) about 2 mg to about 40 mg (e.g., about 5 mg to about 20 mg, about 20 mg to about 30 mg, about 5 mg to about 10 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, or about 40 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor),
GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039; and (d) at least one pharmaceutically acceptable carrier.

In some embodiments, the pharmaceutical compositions provided herein comprise (a) about 5 mg to about 50 mg (e.g., about 20 mg to about 30 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, or about 50 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor),
GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039; and (d) at least one pharmaceutically acceptable carrier.

In some embodiments, the pharmaceutical compositions provided herein comprise (a) about 5 mg to about 20 mg (e.g., about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, or about 20 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor), GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039; and (d) at least one pharmaceutically acceptable carrier.

In some embodiments, the pharmaceutical compositions provided herein comprise (a) about 5 mg to about 10 mg (e.g., about 5 mg, about 5.5 mg, about 6 mg, about 6.5 mg, about 7 mg, about 7.5 mg, about 8 mg, about 8.5 mg, about 9 mg, about 9.5 mg, or about 10 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor), GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039; and (d) at least one pharmaceutically acceptable carrier.

In some embodiments, the pharmaceutical compositions provided herein comprise (a) about 1 mg to about 30 mg (e.g., about 5 mg to about 20 mg, about 20 mg to about 30 mg, about 5 mg to about 10 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, or about 30 mg) of Compound I, a deuterated derivative thereof, or a pharmaceutically acceptable salt of any of the foregoing; (b) optionally at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof; (c) optionally at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor), GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039; and (d) at least one pharmaceutically acceptable carrier.

In some embodiments, the pharmaceutical compositions provided herein comprise (a) about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, (b) Compound II, a deuterated derivative thereof, or a pharmaceutically acceptable salt of any of the foregoing, and (c) at least one pharmaceutically acceptable carrier.

In some embodiments, the pharmaceutical compositions provided herein comprise (a) about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, (b) Compound III, a deuterated derivative thereof, or a pharmaceutically acceptable salt of any of the foregoing, and (c) at least one pharmaceutically acceptable carrier.

In some embodiments, the pharmaceutical compositions provided herein comprise (a) about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, (b) Compound III-d, a deuterated derivative thereof, or a pharmaceutically acceptable salt of any of the foregoing, and (c) at least one pharmaceutically acceptable carrier.

In some embodiments, the pharmaceutical compositions provided herein comprise (a) about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, (b) Compound IV, a deuterated derivative thereof, or a pharmaceutically acceptable salt of any of the foregoing, and (c) at least one pharmaceutically acceptable carrier.

In some embodiments, the pharmaceutical compositions provided herein comprise (a) about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, (b) Compound V, a deuterated derivative thereof, or a pharmaceutically acceptable salt of any of the foregoing, and (c) at least one pharmaceutically acceptable carrier.

In some embodiments, the pharmaceutical compositions provided herein comprise (a) about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, (b) Compound VI, a deuterated derivative thereof, or a pharmaceutically acceptable salt of any of the foregoing, and (c) at least one pharmaceutically acceptable carrier.

In some embodiments, the pharmaceutical compositions provided herein comprise (a) about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) Compound I, a deuterated derivative thereof, or a pharmaceutically acceptable salt of any of the foregoing, (b) Compound VII, a deuterated derivative thereof, or a pharmaceutically acceptable salt of any of the foregoing, and (c) at least one pharmaceutically acceptable carrier.

In some embodiments, the pharmaceutical compositions provided herein comprise (a) about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) Compound I, a deuterated derivative thereof, or a pharmaceutically acceptable salt of any of the foregoing, (b) Compound VIII, a deuterated derivative thereof, or a pharmaceutically acceptable salt of any of the foregoing, and (c) at least one pharmaceutically acceptable carrier.

In some embodiments, the pharmaceutical compositions provided herein comprise (a) about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, (b) Compound IX, a deuterated derivative thereof, or a pharmaceutically acceptable salt of any of the foregoing, and (c) at least one pharmaceutically acceptable carrier.

In some embodiments, the pharmaceutical compositions provided herein comprise (a) about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, (b) Compound X, a deuterated derivative thereof, or a pharmaceutically acceptable salt of any of the foregoing, and (c) at least one pharmaceutically acceptable carrier.

In some embodiments, the pharmaceutical compositions provided herein comprise (a) about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, (b) Compound II, a deuterated derivative thereof, or a pharmaceutically acceptable salt of any of the foregoing, and (c) at least one pharmaceutically acceptable carrier.

In some embodiments, the pharmaceutical compositions provided herein comprise (a) about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, (b) Compound III, a deuterated derivative thereof, or a pharmaceutically acceptable salt of any of the foregoing, and (c) at least one pharmaceutically acceptable carrier.

In some embodiments, the pharmaceutical compositions provided herein comprise (a) about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, (b) Compound III-d, a deuterated derivative thereof, or a pharmaceutically acceptable salt of any of the foregoing, and (c) at least one pharmaceutically acceptable carrier.

In some embodiments, the pharmaceutical compositions provided herein comprise (a) about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, (b) Compound IV, a deuterated derivative thereof, or a pharmaceutically acceptable salt of any of the foregoing, and (c) at least one pharmaceutically acceptable carrier.

In some embodiments, the pharmaceutical compositions provided herein comprise (a) about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, (b) Compound V, a deuterated derivative thereof, or a pharmaceutically acceptable salt of any of the foregoing, and (c) at least one pharmaceutically acceptable carrier.

In some embodiments, the pharmaceutical compositions provided herein comprise (a) about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, (b) Compound VI, a deuterated derivative thereof, or a pharmaceutically acceptable salt of any of the foregoing, and (c) at least one pharmaceutically acceptable carrier.

In some embodiments, the pharmaceutical compositions provided herein comprise (a) about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) Compound I, a deuterated derivative thereof, or a pharmaceutically acceptable salt of any of the foregoing, (b) Compound VII, a deuterated derivative thereof, or a pharmaceutically acceptable salt of any of the foregoing, and (c) at least one pharmaceutically acceptable carrier.

In some embodiments, the pharmaceutical compositions provided herein comprise (a) about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) Compound I, a deuterated derivative thereof, or a pharmaceutically acceptable salt of any of the foregoing, (b) Compound VIII, a deuterated derivative thereof, or a pharmaceutically acceptable salt of any of the foregoing, and (c) at least one pharmaceutically acceptable carrier.

In some embodiments, the pharmaceutical compositions provided herein comprise (a) about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, (b) Compound IX, a deuterated derivative thereof, or a pharmaceutically acceptable salt of any of the foregoing, and (c) at least one pharmaceutically acceptable carrier.

In some embodiments, the pharmaceutical compositions provided herein comprise (a) about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, (b) Compound X, a deuterated derivative thereof, or a pharmaceutically acceptable salt of any of the foregoing, and (c) at least one pharmaceutically acceptable carrier.

In some embodiments, the pharmaceutical compositions provided herein comprise (a) about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, (b) Compound II, a deuterated derivative thereof, or a pharmaceutically acceptable salt of any of the foregoing, (c) Compound V, a deuterated derivative thereof, or a pharmaceutically acceptable salt of any of the foregoing, and (d) at least one pharmaceutically acceptable carrier.

In some embodiments, the pharmaceutical compositions provided herein comprise (a) about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, (b) Compound II, a deuterated derivative thereof, or a pharmaceutically acceptable salt of any of the foregoing, (c) Compound VI, a deuterated derivative thereof, or a pharmaceutically acceptable salt of any of the foregoing, and (d) at least one pharmaceutically acceptable carrier.

In some embodiments, the pharmaceutical compositions provided herein comprise (a) about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, (b) Compound II, a deuterated derivative thereof, or a pharmaceutically acceptable salt of any of the foregoing, (c) Compound VII, a deuterated derivative thereof, or a pharmaceutically acceptable salt of any of the foregoing, and (d) at least one pharmaceutically acceptable carrier.

In some embodiments, the pharmaceutical compositions provided herein comprise (a) about 0.1 mg to about 800 mg (e.g., about 1 mg to about 400 mg, about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg) of Compound I, a deuterated derivative thereof, or a pharmaceutically acceptable salt of any of the foregoing, (b) Compound II, a deuterated derivative thereof, or a pharmaceutically acceptable salt of any of the foregoing, (c) Compound VIII, a deuterated derivative thereof, or a pharmaceutically acceptable salt of any of the foregoing, and (d) at least one pharmaceutically acceptable carrier.

In some embodiments, the pharmaceutical compositions provided herein comprise (a) about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, (b) Compound II, a deuterated derivative thereof, or a pharmaceutically acceptable salt of any of the foregoing, (c) Compound V, a deuterated derivative thereof, or a pharmaceutically acceptable salt of any of the foregoing, and (d) at least one pharmaceutically acceptable carrier.

In some embodiments, the pharmaceutical compositions provided herein comprise (a) about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, (b) Compound II, a deuterated derivative thereof, or a pharmaceutically acceptable salt of any of the foregoing, (c) Compound VI, a deuterated derivative thereof, or a pharmaceutically acceptable salt of any of the foregoing, and (d) at least one pharmaceutically acceptable carrier.

In some embodiments, the pharmaceutical compositions provided herein comprise (a) about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, (b) Compound II, a deuterated derivative thereof, or a pharmaceutically acceptable salt of any of the foregoing, (c) Compound VII, a deuterated derivative thereof, or a pharmaceutically acceptable salt of any of the foregoing, and (d) at least one pharmaceutically acceptable carrier.

In some embodiments, the pharmaceutical compositions provided herein comprise (a) about 1 mg to about 400 mg (e.g., about 5 mg to about 375 mg, about 15 mg to about 350 mg, about 30 mg to about 300 mg, about 50 mg to about 200 mg, about 50 mg to about 175 mg, about 10 mg to about 200 mg, about 20 mg to about 100 mg, about 20 mg to about 30 mg, about 30 mg to about 60 mg, about 1 mg to about 50 mg, about 2 mg to about 40 mg, about 5 mg to about 50 mg, about 5 mg to about 20 mg, about 5 mg to about 10 mg, about 1 mg to about 30 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg) of Compound I, a deuterated derivative thereof, or a pharmaceutically acceptable salt of any of the foregoing, (b) Compound II, a deuterated derivative thereof, or a pharmaceutically acceptable salt of any of the foregoing, (c) Compound VIII, a deuterated derivative thereof, or a pharmaceutically acceptable salt of any of the foregoing, and (d) at least one pharmaceutically acceptable carrier.

Any pharmaceutical composition disclosed herein may comprise at least one pharmaceutically acceptable carrier. In some embodiments, the at least one pharmaceutically acceptable carrier is chosen from pharmaceutically acceptable vehicles and pharmaceutically acceptable adjuvants. In some embodiments, the at least one pharmaceutically acceptable is chosen from pharmaceutically acceptable fillers, disintegrants, surfactants, binders, and lubricants.

The pharmaceutical compositions described herein are useful for treating cystic fibrosis and other CFTR-mediated diseases.

As described above, pharmaceutical compositions disclosed herein may optionally further comprise at least one pharmaceutically acceptable carrier. The at least one pharmaceutically acceptable carrier may be chosen from adjuvants and vehicles. The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include, but are not limited to, ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

### Non-limiting Exemplary Embodiments

1. A method of treating cystic fibrosis comprising administering to a patient in need thereof about 0.1 mg to about 800 mg of Compound I:

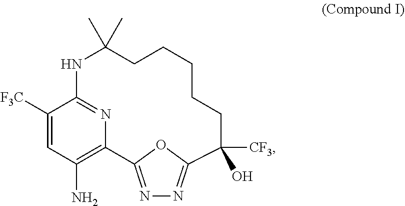 [SMILES: CC1(C)CCCCC[C@](O)(C(F)(F)F)C2=NN=C(O2)c2nc(c(C(F)(F)F)cc2N)N1]

or an equivalent amount of a deuterated derivative or a pharmaceutically acceptable salt thereof daily.
2. The method according to Embodiment 1, wherein the method comprises administering about 1 mg to about 400 mg of Compound I or an equivalent amount of a deuterated derivative or a pharmaceutically acceptable salt thereof daily 3. The method according to Embodiment 1 or Embodiment 2, wherein the method comprises administering about 5 mg to about 375 mg of Compound I or an equivalent amount of a deuterated derivative or a pharmaceutically acceptable salt thereof daily.
4. The method according to any one of Embodiments 1-3, wherein the method comprises administering about 30 mg to about 300 mg of Compound I or an equivalent amount of a deuterated derivative or a pharmaceutically acceptable salt thereof daily.
5. The method according to any one of Embodiments 1-4, wherein the method comprises administering about 50 mg to about 200 mg of Compound I or an equivalent amount of a deuterated derivative or a pharmaceutically acceptable salt thereof daily.
6. The method according to any one of Embodiments 1-5, wherein the method comprises administering about 50 mg to about 175 mg of Compound I or an equivalent amount of a deuterated derivative or a pharmaceutically acceptable salt thereof daily.
7. The method according to any one of Embodiments 1-3, wherein the method comprises administering about 10 mg to about 200 mg of Compound I or an equivalent amount of a deuterated derivative or a pharmaceutically acceptable salt thereof daily.
8. The method according to any one of Embodiments 1-3 or 7, wherein the method comprises administering about 20 mg to about 100 mg of Compound I or an equivalent amount of a deuterated derivative or a pharmaceutically acceptable salt thereof daily
9. The method according to any one of Embodiments 1-3, 7, or 8, wherein the method comprises administering about 30 mg to about 60 mg of Compound I or an equivalent amount of a deuterated derivative or a pharmaceutically acceptable salt thereof daily
10. The method according to Embodiment 1 or Embodiment 2, wherein the method comprises administering about 1 mg to about 50 mg of Compound I or an equivalent amount of a deuterated derivative or a pharmaceutically acceptable salt thereof daily.
11. The method according to any one of Embodiments 1, 2, or 10, wherein the method comprises administering about 2 mg to about 40 mg of Compound I or an equivalent amount of a deuterated derivative or a pharmaceutically acceptable salt thereof daily.
12. The method according to any one of Embodiments 1-3, or 10, wherein the method comprises administering about 5 mg to about 50 mg of Compound I or an equivalent amount of a deuterated derivative or a pharmaceutically acceptable salt thereof daily.
13. The method according to any one of Embodiments 1-3 or 10-12, wherein the method comprises administering about 5 mg to about 20 mg of Compound I or an equivalent amount of a deuterated derivative or a pharmaceutically acceptable salt thereof daily.
14. The method according to any one of Embodiments 1-3 or 10-13, wherein the method comprises administering about 5 mg to about 10 mg of Compound I or an equivalent amount of a deuterated derivative or a pharmaceutically acceptable salt thereof daily.
15. The method according to any one of Embodiments 1, 2 or 10, wherein the method comprises administering about 1 mg to about 30 mg of Compound I or an equivalent amount of a deuterated derivative or a pharmaceutically acceptable salt thereof daily.
16. The method according to any one of Embodiments 1-4, wherein about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg of Compound I or an equivalent amount of a deuterated derivative or a pharmaceutically acceptable salt thereof is administered daily.
17. The method according to any one of Embodiments 1-4, wherein about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, or about 210 mg of Compound I or an equivalent amount of a deuterated derivative or a pharmaceutically acceptable salt thereof is administered daily.
18. The method according to any one of Embodiments 1-4, wherein about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, or about 300 mg of Compound I or an equivalent amount of a deuterated derivative or a pharmaceutically acceptable salt thereof is administered daily.
19. The method according to Embodiment 1, 2 or 10, wherein about 1 mg, about 2 mg, about 3 mg, about 4 mg, about 5 mg, about 7.5 mg, about 10 mg, about 15 mg, about 20 mg, about 25 mg, about 30 mg, about 35 mg, about 40 mg, about 45 mg, or about 50 mg of Compound I or an equivalent amount of a deuterated derivative or a pharmaceutically acceptable salt thereof is administered daily.
20. The method according to Embodiment 1 or Embodiment 2, wherein about 5 mg, about 7.5 mg, about 30 mg, about 35 mg, about 70 mg, about 75 mg, about 150 mg, about 375 mg, about 385 mg, or about 400 mg of Compound I or an equivalent amount of a deuterated derivative or a pharmaceutically acceptable salt thereof is administered daily.
21. The method according to Embodiment 1 or Embodiment 2, wherein about 5 mg, about 7.3 mg, about 34 mg, about 73 mg, about 100 mg, about 150 mg, about 220 mg, or about 390 mg of Compound I or an equivalent amount of a deuterated derivative or a pharmaceutically acceptable salt thereof is administered daily.
22. The method according to any one of Embodiments 1˜4 or 7-9, wherein about 30 mg, about 31 mg, about 32 mg, about 33 mg, about 34 mg, about 35 mg, about 36 mg, about 37 mg, about 38 mg, about 39 mg, about 40 mg, or about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, or about 60 mg of Compound I or an equivalent amount of a deuterated derivative or a pharmaceutically acceptable salt thereof is administered daily.
23. A method of treating cystic fibrosis comprising administering to a patient in need thereof about 0.1 mg to about 800 mg of Compound I or an equivalent amount of a deuterated derivative or a pharmaceutically acceptable salt thereof daily, and wherein the patient is heterozygous and has one F508del mutation.
24. The method according to Embodiment 23, wherein the method comprises administering about 1 mg to about 400 mg of Compound I or an equivalent amount of a deuterated derivative or a pharmaceutically acceptable salt thereof daily.
25. The method according to Embodiment 23 or Embodiment 24, wherein the method comprises administering about 5 mg to about 375 mg of Compound I or an equivalent amount of a deuterated derivative or a pharmaceutically acceptable salt thereof daily.
26. The method according to any one of Embodiments 23-25, wherein the method comprises administering about 30 mg to about 300 mg of Compound I or an equivalent amount of a deuterated derivative or a pharmaceutically acceptable salt thereof daily.
27. The method according to any one of Embodiments 23-26, wherein the method comprises administering about 50 mg to about 200 mg of Compound I or an equivalent amount of a deuterated derivative or a pharmaceutically acceptable salt thereof daily.
28. The method according to any one of Embodiments 23-27, wherein the method comprises administering about 50 mg to about 175 mg of Compound I or an equivalent amount of a deuterated derivative or a pharmaceutically acceptable salt thereof daily.
29. The method according to any one of Embodiments 23-25, wherein the method comprises administering about 10 mg to about 200 mg of Compound I or an equivalent amount of a deuterated derivative or a pharmaceutically acceptable salt thereof daily.
30. The method according to any one of Embodiments 23-25 or 29, wherein the method comprises administering about 20 mg to about 100 mg of Compound I or an equivalent amount of a deuterated derivative or a pharmaceutically acceptable salt thereof daily.
31. The method according to any one of Embodiments 23-25, 29, or 30, wherein the method comprises administering about 30 mg to about 60 mg of Compound I or an equivalent amount of a deuterated derivative or a pharmaceutically acceptable salt thereof daily.
32. The method according to Embodiment 23 or Embodiment 24, wherein the method comprises administering about 1 mg to about 50 mg of Compound I or an equivalent amount of a deuterated derivative or a pharmaceutically acceptable salt thereof daily.
33. The method according to any one of Embodiments 23, 24 or 32, wherein the method comprises administering about 2 mg to about 40 mg of Compound I or an equivalent amount of a deuterated derivative or a pharmaceutically acceptable salt thereof daily.
34. The method according to any one of Embodiments 23-25, or 32, wherein the method comprises administering about 5 mg to about 50 mg of Compound I or an equivalent amount of a deuterated derivative or a pharmaceutically acceptable salt thereof daily.
35. The method according to any one of Embodiments 23-25, or 32-34, wherein the method comprises administering about 5 mg to about 20 mg of Compound I or an equivalent amount of a deuterated derivative or a pharmaceutically acceptable salt thereof daily.
36. The method according to any one of Embodiments 23-25, or 32-35, wherein the method comprises administering about 5 mg to about 10 mg of Compound I or an equivalent amount of a deuterated derivative or a pharmaceutically acceptable salt thereof daily.
37. The method according to any one of Embodiments 23, 24 or 32, wherein the method comprises administering about 1 mg to about 30 mg of Compound I or an equivalent amount of a deuterated derivative or a pharmaceutically acceptable salt thereof daily.
38.The method according to any one of Embodiments 23-26, wherein the method comprises administering about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg of Compound I or an equivalent amount of a deuterated derivative or a pharmaceutically acceptable salt thereof daily.
39. The method according to any one of Embodiments 23-26, wherein the method comprises administering about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, or about 210 mg of Compound I or an equivalent amount of a deuterated derivative or a pharmaceutically acceptable salt thereof daily.
40. The method according to any one of Embodiments 23-26, wherein about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, or about 300 mg of Compound I or an equivalent amount of a deuterated derivative or a pharmaceutically acceptable salt thereof is administered daily.
41. The method according to any one of Embodiments 23, 24 or 32, wherein about 1 mg, about 2 mg, about 3 mg, about 4 mg, about 5 mg, about 7.5 mg, about 10 mg, about 15 mg, about 20 mg, about 25 mg, about 30 mg, about 35 mg, about 40 mg, about 45 mg, or about 50 mg of Compound I or an equivalent amount of a deuterated derivative or a pharmaceutically acceptable salt thereof is administered daily.
42. The method according to Embodiment 23 or Embodiment 24, wherein about 5 mg, about 7.5 mg, about 30 mg, about 35 mg, about 70 mg, about 75 mg, about 150 mg, about 375 mg, about 385 mg, or about 400 mg of Compound I or an equivalent amount of a deuterated derivative or a pharmaceutically acceptable salt thereof is administered daily.
43. The method according to Embodiment 23 or Embodiment 24, wherein about 5 mg, about 7.3 mg, about 34 mg, about 73 mg, about 100 mg, about 150 mg, about 220 mg, or about 390 mg of Compound I or an equivalent amount of a deuterated derivative or a pharmaceutically acceptable salt thereof is administered daily.
44. The method according to any one of Embodiments 23-26 or 29-31, wherein the method comprises administering about 30 mg, about 31 mg, about 32 mg, about 33 mg, about 34 mg, about 35 mg, about 36 mg, about 37 mg, about 38 mg, about 39 mg, about 40 mg, or about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, or about 60 mg of Compound I or an equivalent amount of a deuterated derivative or a pharmaceutically acceptable salt thereof daily.
45. The method according to any one of Embodiments 1-44, wherein Compound I or a deuterated derivative or a pharmaceutically acceptable salt thereof is administered as a single dose, once daily.
46. The method according to any one of Embodiments 1-44, wherein Compound I or a deuterated derivative or a pharmaceutically acceptable salt thereof is administered in two doses daily.
47. The method according to any one of Embodiments 1-46, further comprising administering one or more additional therapeutic agent(s).
48. The method according to Embodiment 47, wherein the one or more additional therapeutic agent(s) comprise(s) a compound with CFTR-modulating activity or a deuterated derivative or a pharmaceutically acceptable salt thereof.
49. The method according to Embodiment 47 or Embodiment 48, wherein the one or more additional therapeutic agent(s) comprise(s) a CFTR corrector.
50. The method according to any one of Embodiments 47-49, wherein the one or more additional therapeutic agent(s) comprise(s) a compound selected from:
(a) (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl) cyclopropanecarboxamide (Compound II):

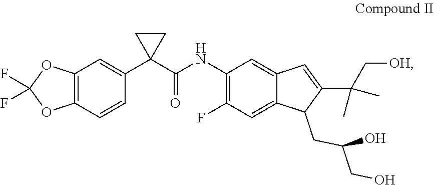 [SMILES: CC(C)(CO)C1=Cc2cc(NC(=O)C3(c4ccc5c(c4)OC(F)(F)O5)CC3)c(F)cc2C1C[C@@H](O)CO]

(b) 3-(6-(1-(2,2-difluorobenzo[d][1,3]dioxol-5-yl) cyclopropanecarboxamido)-3-methylpyridin-2-yl)benzoic acid (Compound IV):

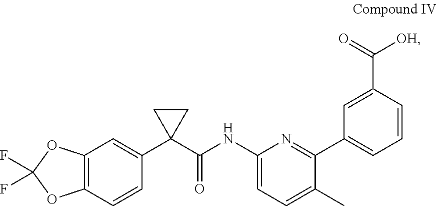 [SMILES: Cc1ccc(NC(=O)C2(c3ccc4c(c3)OC(F)(F)O4)CC2)nc1-c1cccc(C(=O)O)c1]

(c) N-(1,3-dimethylpyrazol-4-yl)sulfonyl-6-[3-(3,3,3-trifluoro-2,2-dimethyl-propoxy) pyrazol-1-yl]-2-[(4S)-2,2,4-trimethylpyrrolidin-1-yl]pyridine-3-carboxamide (Compound V):

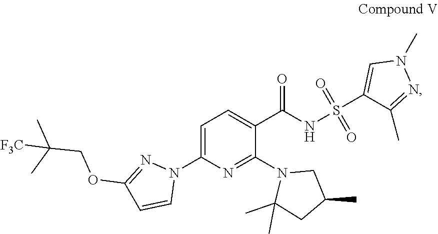 [SMILES: Cc1nn(C)cc1S(=O)(=O)NC(=O)c1ccc(-n2ccc(OCC(C)(C)C(F)(F)F)n2)nc1N1C[C@@H](C)CC1(C)C]

(d) N-(benzenesulfonyl)-6-[3-[2-[1-(trifluoromethyl)cyclopropyl]ethoxy]pyrazol-1-yl]-2-[(4S)-2,2,4-trimethylpyrrolidin-1-yl]pyridine-3-carboxamide (Compound VI):

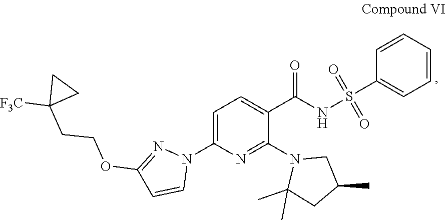 [SMILES: C[C@@H]1CN(c2nc(-n3ccc(OCCC4(C(F)(F)F)CC4)n3)ccc2C(=O)NS(=O)(=O)c2ccccc2)C(C)(C)C1]

(e) (14S)-8-[3-(2-{dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1 (22),5,7,9,19 (23),20-hexaene-2,2,4-trione (Compound VII):

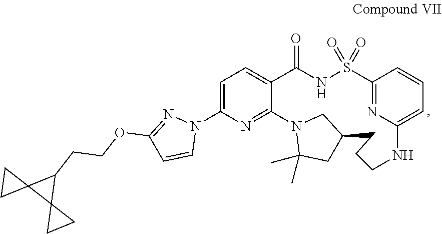 [SMILES: CC1(C)C[C@@H]2CCCNc3cccc(n3)S(=O)(=O)NC(=O)c3ccc(-n4ccc(OCCC5C6(CC6)C56CC6)n4)nc3N1C2]

(f) (11R)-6-(2,6-dimethylphenyl)-11-(2-methylpropyl)-12-{spiro[2.3]hexan-5-yl}-9-oxa-276-thia-3,5,12,19-tetraazatricyclo[12.3.1.14,8]nonadeca-1 (17),4 (19),5,7,14 (18), 15-hexaene-2,2,13-trione (Compound VIII):

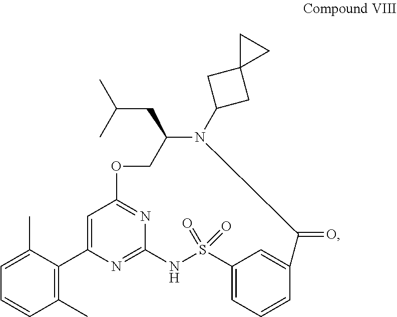 [SMILES: Cc1cccc(C)c1-c1cc2nc(n1)NS(=O)(=O)c1cccc(c1)C(=O)N(C1CC3(CC3)C1)[C@H](CC(C)C)CO2]

(g) N-(benzenesulfonyl)-6-(3-fluoro-5-isobutoxy-phenyl)-2-[(4S)-2,2,4-trimethylpyrrolidin-1-yl]pyridine-3-carboxamide (Compound IX):

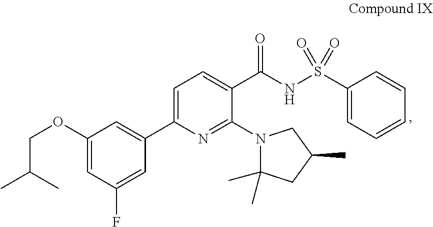 [SMILES: CC(C)COc1cc(F)cc(-c2ccc(C(=O)NS(=O)(=O)c3ccccc3)c(N3C[C@@H](C)CC3(C)C)n2)c1]

and
(h) N-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide (Compound X):

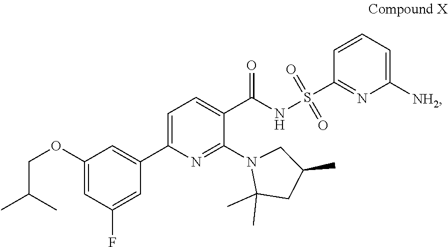 [SMILES: CC(C)COc1cc(F)cc(-c2ccc(C(=O)NS(=O)(=O)c3cccc(N)n3)c(N3C[C@@H](C)CC3(C)C)n2)c1]

and deuterated derivatives and pharmaceutically acceptable salts thereof.
51. The method according to any one of Embodiments 47-50, wherein the one or more additional therapeutic agent(s) comprise(s) at least one compound selected from PTI-428, ABBV-2222, ABBV-2851, GLPG2737, ABBV-3221, ABBV-3748, ABBV-3903, ABBV-119, ABBV-2851, FDL-169, ARN5562, ARN21586, ARN22081, ARN22652, ARN23765, ARN23766, and PTI-801.
52. The method according to any one of Embodiments 47-51, wherein the one or more additional therapeutic agent(s) comprise(s) a CFTR potentiator enhancer.
53. The method according to any one of Embodiments 47-52, wherein the one or more additional therapeutic agent(s) comprise(s) ASP-11.
54. The method according to any one of Embodiments 47-53, wherein the one or more additional therapeutic agent(s) comprise(s) a CFTR potentiator.
55. The method according to any one of Embodiments 47-54, wherein the one or more additional therapeutic agent(s) comprise(s) a compound selected from:
(a) N-(5-hydroxy-2,4-di-tert-butyl-phenyl)-4-oxo-1H-quinoline-3-carboxamide (Compound III):

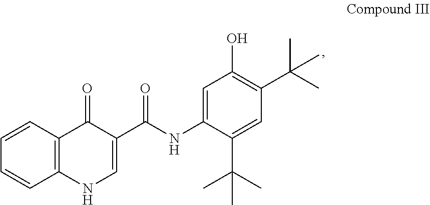 [SMILES: CC(C)(C)c1cc(C(C)(C)C)c(NC(=O)c2c[nH]c3ccccc3c2=O)cc1O]

and
(b) N-(2-(tert-butyl)-5-hydroxy-4-(2-(methyl-d3) propan-2-yl-1,1,1,3,3,3-d6)phenyl)-4-oxo-1,4-dihydroquinoline-3-carboxamide (Compound III-d):

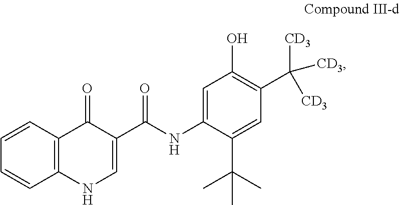 [SMILES: [2H]C([2H])([2H])C(c1cc(C(C)(C)C)c(NC(=O)c2c[nH]c3ccccc3c2=O)cc1O)(C([2H])([2H])[2H])C([2H])([2H])[2H]]

and deuterated derivatives and pharmaceutically acceptable salts thereof.
56. The method according to any one of Embodiments 47-55, wherein the one or more additional therapeutic agent(s) comprise(s) at least one compound selected from FDL-176, PTI-808, GLPG1837/ABBV-974, GLPG2451/ABBV-2451, QBW251 (Icenticaftor), GLPG3067/ABBV-3067 (Navocaftor), and ABBV-191.
57. The method according to any one of Embodiments 47-56, wherein the one or more additional therapeutic agent(s) comprise(s) a CFTR amplifier.
58. The method according to any one of Embodiments 47-57, wherein the one or more additional therapeutic agent(s) comprise(s) PTI-428.
59. The method according to any one of Embodiments 47-58, wherein the one or more additional therapeutic agent(s) comprise(s) a CFTR readthrough agent.
60. The method according to any one of Embodiments 47-59, wherein the one or more additional therapeutic agent(s) comprise(s) ELX-02.
61. The method according to any one of Embodiments 47-60, wherein the one or more additional therapeutic agent(s) comprise(s) a nucleic acid therapy.
62. The method according to any one of Embodiments 47-61, wherein the one or more additional therapeutic agent(s) comprise(s) at least one agent selected from MRT5005, Lunar-CF, and RCT223.
63. The method according to any one of Embodiments 47-62, wherein the one or more additional therapeutic agent(s) comprise(s) an ENaC inhibitor.
64. The method according to any one of Embodiments 47-63, wherein the one or more additional therapeutic agent(s) comprise(s) amiloride, ETD001, CF552, GS-9411, GS-5737, P-1037 (VX-371), P-1055 (VX-551), AZD5634, SPX-101, Ionis-ENaC-2.5 Rx, BI 1265162, or ARO-ENaC1001.
65. The method according to any one of Embodiments 47-64, wherein the one or more additional therapeutic agent(s) comprise(s) a TMEM16A modulator.
66. The method according to any one of Embodiments 47-65, wherein the one or more additional therapeutic agent(s) comprise(s) ETD002.
67. The method according to any one of Embodiments 47-66, wherein the one or more additional therapeutic agent(s) comprise(s) a GPR39 agonist.
68. The method according to any one of Embodiments 47-67, wherein the one or more additional therapeutic agent(s) comprise(s) DS-1039.
69. A pharmaceutical composition comprising about 0.1 mg to about 800 mg of Compound I or an equivalent amount of a deuterated derivative or a pharmaceutically acceptable salt thereof.
70. The pharmaceutical composition according to Embodiment 69, wherein the composition comprises about 1 mg to about 400 mg of Compound I or an equivalent amount of a deuterated derivative or a pharmaceutically acceptable salt thereof.
71. The pharmaceutical composition according to Embodiment 69 and Embodiment 70, wherein the composition comprises about 5 mg to about 375 mg of Compound I or an equivalent amount of a deuterated derivative or a pharmaceutically acceptable salt thereof.
72. The pharmaceutical composition according to any one of Embodiments 69-71, wherein the composition comprises about 30 mg to about 300 mg of Compound I or an equivalent amount of a deuterated derivative or a pharmaceutically acceptable salt thereof.
73. The pharmaceutical composition according to any one of Embodiments 69-72, wherein the composition comprises about 50 mg to about 200 mg of Compound I or an equivalent amount of a deuterated derivative or a pharmaceutically acceptable salt thereof.
74. The pharmaceutical composition according to any one of Embodiments 69-73, wherein the composition comprises about 50 mg to about 175 mg of Compound I or an equivalent amount of a deuterated derivative or a pharmaceutically acceptable salt thereof.
75. The pharmaceutical composition according to any one of Embodiments 69-71, wherein the composition comprises about 10 mg to about 200 mg of Compound I or an equivalent amount of a deuterated derivative or a pharmaceutically acceptable salt thereof.
76. The pharmaceutical composition according to any one of Embodiments 69-71 or 75, wherein the composition comprises about 20 mg to about 100 mg of Compound I or an equivalent amount of a deuterated derivative or a pharmaceutically acceptable salt thereof.
77. The pharmaceutical composition according to any one of Embodiments 69-71, 75, or 76, wherein the composition comprises about 30 mg to about 60 mg of Compound I or an equivalent amount of a deuterated derivative or a pharmaceutically acceptable salt thereof.
78. The pharmaceutical composition according to Embodiment 69 or Embodiment 70, wherein the composition comprises about 1 mg to about 50 mg of Compound I or an equivalent amount of a deuterated derivative or a pharmaceutically acceptable salt thereof.
79. The pharmaceutical composition according to any one of Embodiments 69, 70, or 78, wherein the composition comprises about 2 mg to about 40 mg of Compound I or an equivalent amount of a deuterated derivative or a pharmaceutically acceptable salt thereof.
80. The pharmaceutical composition according to any one of Embodiments 69-71, or 78, wherein the composition comprises about 5 mg to about 50 mg of Compound I or an equivalent amount of a deuterated derivative or a pharmaceutically acceptable salt thereof.
81. The pharmaceutical composition according to any one of Embodiments 69-71, or 78-80, wherein the composition comprises about 5 mg to about 20 mg of Compound I or an equivalent amount of a deuterated derivative or a pharmaceutically acceptable salt thereof.
82. The pharmaceutical composition according to any one of Embodiments 69-71, or 78-81, wherein the composition comprises about 5 mg to about 10 mg of Compound I or an equivalent amount of a deuterated derivative or a pharmaceutically acceptable salt thereof.
83. The pharmaceutical composition according to any one of Embodiments 69, 70 or 78, wherein the composition comprises about 1 mg to about 30 mg of Compound I or an equivalent amount of a deuterated derivative or a pharmaceutically acceptable salt thereof.
84. The pharmaceutical composition according to any one of Embodiments 69-72, wherein the composition comprises about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg of Compound I or an equivalent amount of a deuterated derivative or a pharmaceutically acceptable salt thereof.
85. The pharmaceutical composition according to any one of Embodiments 69-72, wherein the composition comprises about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, or about 210 mg of Compound I or an equivalent amount of a deuterated derivative or a pharmaceutically acceptable salt thereof.
86. The pharmaceutical composition according to any one of Embodiments 69-72, wherein the composition comprises about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, or about 300 mg of Compound I or an equivalent amount of a deuterated derivative or a pharmaceutically acceptable salt thereof.
87. The pharmaceutical composition according to any one of Embodiments 69, 70, or 78, wherein the composition comprises about 1 mg, about 2 mg, about 3 mg, about 4 mg, about 5 mg, about 7.5 mg, about 10 mg, about 15 mg, about 20 mg, about 25 mg, about 30 mg, about 35 mg, about 40 mg, about 45 mg, or about 50 mg of Compound I or an equivalent amount of a deuterated derivative or a pharmaceutically acceptable salt thereof.
88. The pharmaceutical composition according to Embodiment 69 or Embodiment 70, wherein the composition comprises about 5 mg, about 7.5 mg, about 30 mg, about 35 mg, about 70 mg, about 75 mg, about 150 mg, about 375 mg, about 385 mg, or about 400 mg of Compound I or an equivalent amount of a deuterated derivative or a pharmaceutically acceptable salt thereof.
89. The pharmaceutical composition according to Embodiment 69 or Embodiment 70, wherein the composition comprises about 5 mg, about 7.3 mg, about 34 mg, about 73 mg, about 100 mg, about 150 mg, about 220 mg, or about 387 mg of Compound I or an equivalent amount of a deuterated derivative or a pharmaceutically acceptable salt thereof.
90. The pharmaceutical composition according to any one of Embodiments 69-72, or 75-77, wherein the composition comprises about 30 mg, about 31 mg, about 32 mg, about 33 mg, about 34 mg, about 35 mg, about 36 mg, about 37 mg, about 38 mg, about 39 mg, about 40 mg, or about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, or about 60 mg of Compound I or an equivalent amount of a deuterated derivative or a pharmaceutically acceptable salt thereof.
91. The pharmaceutical composition according to any one of Embodiments 69-90, wherein the composition further comprises one or more additional therapeutic agent(s).
92. The pharmaceutical composition according to Embodiment 91, wherein the one or more additional therapeutic agent(s) comprise(s) a compound with CFTR-modulating activity or a deuterated derivative or a pharmaceutically acceptable salt thereof.
93. The pharmaceutical composition according to Embodiment 91 or Embodiment 92, wherein the one or more additional therapeutic agent(s) comprise(s) a CFTR corrector.
94. The pharmaceutical composition according to any one of Embodiments 91-93, wherein the one or more additional therapeutic agent(s) comprise(s) a compound selected from:
(a) Compound II,
(b) Compound IV,
(c) Compound V,
(d) Compound VI,
(e) Compound VII,
(f) Compound VIII,
(g) Compound IX, and
(i) Compound X, and deuterated derivatives and pharmaceutically acceptable salts thereof.
95. The pharmaceutical composition according to any one of Embodiments 91-94, wherein the one or more additional therapeutic agent(s) comprise(s) at least one compound selected from PTI-428, ABBV-2222, ABBV-2851, GLPG2737, ABBV-3221, ABBV-3748, ABBV-3903, ABBV-119, ABBV-2851, FDL-169, ARN5562, ARN21586, ARN22081, ARN22652, ARN23765, ARN23766, and PTI-801.
96. The pharmaceutical composition according to any one of Embodiments 91-95, wherein the one or more additional therapeutic agent(s) comprise(s) a CFTR potentiator enhancer.
97. The pharmaceutical composition according to any one of Embodiments 91-96, wherein the one or more additional therapeutic agent(s) comprise(s) ASP-11.
98. The pharmaceutical composition according to any one of Embodiments 91-97, wherein the one or more additional therapeutic agent(s) comprise(s) a CFTR potentiator.
99. The pharmaceutical composition according to any one of Embodiments 91-98, wherein the one or more additional therapeutic agent(s) comprise(s) a compound selected from Compound III, Compound III-d, and deuterated derivatives and pharmaceutically acceptable salts thereof.
100. The pharmaceutical composition according to any one of Embodiments 91-99, wherein the one or more additional therapeutic agent(s) comprise(s) at least one compound selected from FDL-176, PTI-808, GLPG1837/ABBV-974, GLPG2451/ABBV-2451, QBW251 (Icenticaftor), GLPG3067/ABBV-3067 (Navocaftor), and ABBV-191.
101. The pharmaceutical composition according to any one of Embodiments 91-100, wherein the one or more additional therapeutic agent(s) comprise(s) a CFTR amplifier. The pharmaceutical composition according to any one of Embodiments 91-101, 102, wherein the one or more additional therapeutic agent(s) comprise(s) PTI-428.
103. The pharmaceutical composition according to any one of Embodiments 91-102, wherein the one or more additional therapeutic agent(s) comprise(s) a CFTR readthrough agent.
104. The pharmaceutical composition according to any one of Embodiments 91-103, wherein the one or more additional therapeutic agent(s) comprise(s) ELX-02.
105. The pharmaceutical composition according to any one of Embodiments 91-104, wherein the one or more additional therapeutic agent(s) comprise(s) a nucleic acid therapy.
106. The pharmaceutical composition according to any one of Embodiments 91-105, wherein the one or more additional therapeutic agent(s) comprise(s) at least one agent selected from MRT5005, Lunar-CF, and RCT223.
107. The pharmaceutical composition according to any one of Embodiments 91-106, wherein the one or more additional therapeutic agent(s) comprise(s) an ENaC inhibitor.
108. The pharmaceutical composition according to any one of Embodiments 91-107, wherein the one or more additional therapeutic agent(s) comprise(s) amiloride, ETD001, CF552, GS-9411, GS-5737, P-1037 (VX-371), P-1055 (VX-551), AZD5634, SPX-101, Ionis-ENaC-2.5 Rx, BI 1265162, or ARO-ENaC1001.
109. The pharmaceutical composition according to any one of Embodiments 91-108, wherein the one or more additional therapeutic agent(s) comprise(s) a TMEM16A modulator.
110. The pharmaceutical composition according to any one of Embodiments 91-109, wherein the one or more additional therapeutic agent(s) comprise(s) ETD002.
111. The pharmaceutical composition according to any one of Embodiments 91-110, wherein the one or more additional therapeutic agent(s) comprise(s) a GPR39 agonist.
112. The pharmaceutical composition according to any one of Embodiments 91-111, wherein the one or more additional therapeutic agent(s) comprise(s) DS-1039.
113. Compound I or a deuterated derivative or pharmaceutically acceptable salt thereof, or a pharmaceutical composition according to any one of Embodiments 68-112, for use in a method of treating cystic fibrosis in a patient comprising administering about 0.1 mg to about 800 mg Compound I or an equivalent amount of a deuterated derivative or pharmaceutically acceptable salt thereof daily.
114. The compound, deuterated derivative, salt, or composition for use according to Embodiment 113, wherein the method comprises administering about 1 mg to about 400 mg of Compound I or an equivalent amount of a deuterated derivative or pharmaceutically acceptable salt thereof daily.
115. The compound, deuterated derivative, salt, or composition for use according to Embodiment 113 or Embodiment 114, wherein the method comprises administering about 5 mg to about 375 mg of Compound I or an equivalent amount of a deuterated derivative or pharmaceutically acceptable salt thereof daily.
116. The compound, deuterated derivative, salt, or composition for use according to any one of Embodiments 113-115, wherein the method comprises administering about 30 mg to about 300 mg of Compound I or an equivalent amount of a deuterated derivative or pharmaceutically acceptable salt thereof daily.
117. The compound, deuterated derivative, salt, or composition for use according to any one of Embodiments 113-116, wherein the method comprises administering about 50 mg to about 200 mg of Compound I or an equivalent amount of a deuterated derivative or pharmaceutically acceptable salt thereof daily.
118. The compound, deuterated derivative, salt, or composition for use according to any one of Embodiments 113-117, wherein the method comprises administering about 50 mg to about 175 mg of Compound I or an equivalent amount of a deuterated derivative or pharmaceutically acceptable salt thereof daily.
119. The compound, deuterated derivative, salt, or composition for use according to any one of Embodiments 113-115, wherein the method comprises administering about 10 mg to about 200 mg of Compound I or an equivalent amount of a deuterated derivative or pharmaceutically acceptable salt thereof daily.
120. The compound, deuterated derivative, salt, or composition for use according to any one of Embodiments 113-115 or 119, wherein the method comprises administering about 20 mg to about 100 mg of Compound I or an equivalent amount of a deuterated derivative or pharmaceutically acceptable salt thereof daily.
121. The compound, deuterated derivative, salt, or composition for use according to any one of Embodiments 113-115, 119, or 120, wherein the method comprises administering about 30 mg to about 60 mg of Compound I or an equivalent amount of a deuterated derivative or pharmaceutically acceptable salt thereof daily.
122. The compound, deuterated derivative, salt, or composition for use according to Embodiment 113 or Embodiment 114, wherein the method comprises administering about 1 mg to about 50 mg of Compound I or an equivalent amount of a deuterated derivative or pharmaceutically acceptable salt thereof daily.
123. The compound, deuterated derivative, salt, or composition for use according to any one of Embodiments 113, 114, or 122, wherein the method comprises administering about 2 mg to about 40 mg of Compound I or an equivalent amount of a deuterated derivative or pharmaceutically acceptable salt thereof daily.
124. The compound, deuterated derivative, salt, or composition for use according to any one of Embodiments 113-115, or 122, wherein the method comprises administering about 5 mg to about 50 mg of Compound I or an equivalent amount of a deuterated derivative or pharmaceutically acceptable salt thereof daily.
125. The compound, deuterated derivative, salt, or composition for use according to any one of Embodiments 113-115, or 122-124, wherein the method comprises administering about 5 mg to about 20 mg of Compound I or an equivalent amount of a deuterated derivative or pharmaceutically acceptable salt thereof daily.
126. The compound, deuterated derivative, salt, or composition for use according to any one of Embodiments 113-115, or 122-125, wherein the method comprises administering about 5 mg to about 10 mg of Compound I or an equivalent amount of a deuterated derivative or pharmaceutically acceptable salt thereof daily.
127. The compound, deuterated derivative, salt, or composition for use according to any one of Embodiments 113, 114, or 122, wherein the method comprises administering about 1 mg to about 30 mg of Compound I or an equivalent amount of a deuterated derivative or pharmaceutically acceptable salt thereof daily.
128. The compound, deuterated derivative, salt, or composition for use according to any one of Embodiments 113-116, wherein the method comprises administering about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg of Compound I or an equivalent amount of a deuterated derivative or pharmaceutically acceptable salt thereof daily.
129. The compound, deuterated derivative, salt, or composition for use according to any one of Embodiments 113-116, wherein the method comprises administering about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, or about 210 mg of Compound I or an equivalent amount of a deuterated derivative or pharmaceutically acceptable salt thereof daily.
130. The compound, deuterated derivative, salt, or composition for use according to any one of Embodiments 113-116, wherein the method comprises administering 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, or about 300 mg of Compound I or an equivalent amount of a deuterated derivative or pharmaceutically acceptable salt thereof daily.
131. The compound, deuterated derivative, salt, or composition for use according to any one of Embodiments 113, 114, or 122, wherein the method comprises administering about 1 mg, about 2 mg, about 3 mg, about 4 mg, about 5 mg, about 7.5 mg, about 10 mg, about 15 mg, about 20 mg, about 25 mg, about 30 mg, about 35 mg, about 40 mg, about 45 mg, or about 50 mg of Compound I or an equivalent amount of a deuterated derivative or pharmaceutically acceptable salt thereof daily.
132. The compound, deuterated derivative, salt, or composition for use according to Embodiment 113 or Embodiment 114, wherein the method comprises administering about 5 mg, about 7.5 mg, about 30 mg, about 35 mg, about 70 mg, about 75 mg, about 150 mg, about 375 mg, about 385 mg, or about 400 mg of Compound I or an equivalent amount of a deuterated derivative or pharmaceutically acceptable salt thereof daily.
133. The compound, deuterated derivative, salt, or composition for use according to Embodiment 113 or Embodiment 114, wherein the method comprises administering about 5 mg, about 7.3 mg, about 34 mg, about 73 mg, about 100 mg, about 150 mg, about 220 mg, or about 390 mg of Compound I or an equivalent amount of a deuterated derivative or pharmaceutically acceptable salt thereof daily.
134. The compound, deuterated derivative, salt, or composition for use according to any one of Embodiments 113-116, or 119-121, wherein the method comprises administering about 30 mg, about 31 mg, about 32 mg, about 33 mg, about 34 mg, about 35 mg, about 36 mg, about 37 mg, about 38 mg, about 39 mg, about 40 mg, or about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, or about 60 mg of Compound I or an equivalent amount of a deuterated derivative or pharmaceutically acceptable salt thereof daily.
135. Compound I or a deuterated derivative or a pharmaceutically acceptable salt thereof, or a pharmaceutical composition according to any one of Embodiments 68-112, for use in a method of treating cystic fibrosis in a patient comprising administering about 1 mg to about 400 mg Compound I or an equivalent amount of a deuterated derivative or pharmaceutically acceptable salt thereof daily, and wherein the patient is heterozygous and has one F508del mutation.
136. The compound, deuterated derivative, salt, or composition for use according to Embodiment 135, wherein the method comprises administering about 1 mg to about 400 mg of Compound I or an equivalent amount of a deuterated derivative or pharmaceutically acceptable salt thereof daily.
137. The compound, deuterated derivative, salt, or composition for use according to Embodiment 135 or Embodiment 136, wherein the method comprises administering about 5 mg to about 375 mg of Compound I or an equivalent amount of a deuterated derivative or pharmaceutically acceptable salt thereof daily.
138. The compound, deuterated derivative, salt, or composition for use according to any one of Embodiments 135-137, wherein the method comprises administering about 30 mg to about 300 mg of Compound I or an equivalent amount of a deuterated derivative or pharmaceutically acceptable salt thereof daily.
139. The compound, deuterated derivative, salt, or composition for use according to any one of Embodiments 135-138, wherein the method comprises administering about 50 mg to about 200 mg of Compound I or an equivalent amount of a deuterated derivative or pharmaceutically acceptable salt thereof daily.
140. The compound, deuterated derivative, salt, or composition for use according to any one of Embodiments 135-139, wherein the method comprises administering about 50 mg to about 175 mg of Compound I or an equivalent amount of a deuterated derivative or pharmaceutically acceptable salt thereof daily.
141. The compound, deuterated derivative, salt, or composition for use according to any one of Embodiments 135-137, wherein the method comprises administering about 10 mg to about 200 mg of Compound I or an equivalent amount of a deuterated derivative or pharmaceutically acceptable salt thereof daily.
142. The compound, deuterated derivative, salt, or composition for use according to any one of Embodiments 135-137, or 141, wherein the method comprises administering about 20 mg to about 100 mg of Compound I or an equivalent amount of a deuterated derivative or pharmaceutically acceptable salt thereof daily.
143. The compound, deuterated derivative, salt, or composition for use according to any one of Embodiments 135-137, 141, or 142, wherein the method comprises administering about 30 mg to about 60 mg of Compound I or an equivalent amount of a deuterated derivative or pharmaceutically acceptable salt thereof daily.
144. The compound, deuterated derivative, salt, or composition for use according to Embodiment 135 or Embodiment 136, wherein the method comprises administering about 1 mg to about 50 mg of Compound I or an equivalent amount of a deuterated derivative or pharmaceutically acceptable salt thereof daily.
145. The compound, deuterated derivative, salt, or composition for use according to any one of Embodiments 135, 136, or 144, wherein the method comprises administering about 2 mg to about 40 mg of Compound I or an equivalent amount of a deuterated derivative or pharmaceutically acceptable salt thereof daily.
146. The compound, deuterated derivative, salt, or composition for use according to any one of Embodiments 135-137, or 144, wherein the method comprises administering about 5 mg to about 50 mg of Compound I or an equivalent amount of a deuterated derivative or pharmaceutically acceptable salt thereof daily.
147. The compound, deuterated derivative, salt, or composition for use according to any one of Embodiments 135-137, or 144-146, wherein the method comprises administering about 5 mg to about 20 mg of Compound I or an equivalent amount of a deuterated derivative or pharmaceutically acceptable salt thereof daily.
148. The compound, deuterated derivative, salt, or composition for use according to any one of Embodiments 135-137, or 144-147, wherein the method comprises administering about 5 mg to about 10 mg of Compound I or an equivalent amount of a deuterated derivative or pharmaceutically acceptable salt thereof daily.
149. The compound, deuterated derivative, salt, or composition for use according to any one of Embodiments 135, 136, or 144, wherein the method comprises administering about 1 mg to about 30 mg of Compound I or an equivalent amount of a deuterated derivative or pharmaceutically acceptable salt thereof daily.
150. The compound, deuterated derivative, salt, or composition for use according to any one of Embodiments 135-138, wherein the method comprises administering about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg of Compound I or an equivalent amount of a deuterated derivative or pharmaceutically acceptable salt thereof daily.
151. The compound, deuterated derivative, salt, or composition for use according to any one of Embodiments 135-138, wherein the method comprises administering about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, or about 210 mg of Compound I or an equivalent amount of a deuterated derivative or pharmaceutically acceptable salt thereof daily.
152. The compound, deuterated derivative, salt, or composition for use according to any one of Embodiments 135-138, wherein the method comprises administering about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, or about 300 mg of Compound I or an equivalent amount of a deuterated derivative or pharmaceutically acceptable salt thereof daily.
153. The compound, deuterated derivative, salt, or composition for use according to any one of Embodiments 135, 136, or 144, wherein the method comprises administering about 1 mg, about 2 mg, about 3 mg, about 4 mg, about 5 mg, about 7.5 mg, about 10 mg, about 15 mg, about 20 mg, about 25 mg, about 30 mg, about 35 mg, about 40 mg, about 45 mg, or about 50 mg of Compound I or an equivalent amount of a deuterated derivative or pharmaceutically acceptable salt thereof daily.
154. The compound, deuterated derivative, salt, or composition for use according to Embodiment 135 or Embodiment 136, wherein the method comprises administering about 5 mg, about 7.5 mg, about 30 mg, about 35 mg, about 70 mg, about 75 mg, about 150 mg, about 375 mg, about 385 mg, or about 400 mg of Compound I or an equivalent amount of a deuterated derivative or pharmaceutically acceptable salt thereof.
155. The compound, deuterated derivative, salt, or composition for use according to Embodiment 135 or Embodiment 136, wherein the method comprises administering about 5 mg, about 7.3 mg, about 34 mg, about 73 mg, about 100 mg, about 150 mg, about 220 mg, or about 390 mg of Compound I or an equivalent amount of a deuterated derivative or pharmaceutically acceptable salt thereof.
156. The compound, deuterated derivative, salt, or composition for use according to any one of Embodiments 135-138, or 141-143, wherein the method comprises administering about 30 mg, about 31 mg, about 32 mg, about 33 mg, about 34 mg, about 35 mg, about 36 mg, about 37 mg, about 38 mg, about 39 mg, about 40 mg, or about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, or about 60 mg of Compound I or an equivalent amount of a deuterated derivative or pharmaceutically acceptable salt thereof daily.
157. The compound, deuterated derivative, salt, or composition for use according to any one of Embodiments 113-156, wherein the method comprises administering Compound I or a deuterated derivative or pharmaceutically acceptable salt thereof as a single dose, once daily.
158. The compound, deuterated derivative, salt, or composition for use according to any one of Embodiments 113-156, wherein the method comprises administering Compound I or a deuterated derivative or pharmaceutically acceptable salt thereof in two doses daily.
159. The compound, deuterated derivative, salt, or composition for use according to any one of Embodiments 113-158, wherein the method further comprises administering one or more additional therapeutic agent(s).
160. The compound, deuterated derivative, salt, or composition for use according to Embodiment 159, wherein the one or more additional therapeutic agent(s) comprise(s) a compound with CFTR-modulating activity or a deuterated derivative or pharmaceutically acceptable salt thereof.
161. The compound, deuterated derivative, salt, or composition for use according to Embodiment 159 or Embodiment 160, wherein the one or more additional therapeutic agent(s) comprise(s) a CFTR corrector.
162. The compound, deuterated derivative, salt, or composition for use according to any one of Embodiments 159-161, wherein the one or more additional therapeutic agent(s) comprise(s) a compound selected from:
(a) Compound II,
(b) Compound IV,
(c) Compound V,
(d) Compound VI,
(e) Compound VII,
(f) Compound VIII,
(g) Compound IX, and
(h) Compound X, and deuterated derivatives thereof and pharmaceutically acceptable salts of any of the foregoing.
163. The compound, deuterated derivative, salt, or composition for use according to any one of Embodiments 159-162, wherein the one or more additional therapeutic agent(s) comprise(s) at least one compound selected from PTI-428, ABBV-2222, ABBV-2851, GLPG2737, ABBV-3221, ABBV-3748, ABBV-3903, ABBV-119, ABBV-2851, FDL-169, ARN5562, ARN21586, ARN22081, ARN22652, ARN23765, ARN23766, and PTI-801.
164. The compound, deuterated derivative, salt, or composition for use according to any one of Embodiments 159-163, wherein the one or more additional therapeutic agent(s) comprise(s) a CFTR potentiator enhancer.
165. The compound, deuterated derivative, salt, or composition for use according to any one of Embodiments 159-164, wherein the one or more additional therapeutic agent(s) comprise(s) ASP-11.
166. The compound, deuterated derivative, salt, or composition for use according to any one of Embodiments 159-165, wherein the one or more additional therapeutic agent(s) comprise(s) a CFTR potentiator.
167. The compound, deuterated derivative, salt, or composition for use according to any one of Embodiments 159-166, wherein the one or more additional therapeutic agent(s) comprise(s) a compound selected from Compound III, Compound III-d, and pharmaceutically acceptable salts and deuterated derivatives thereof.
168. The compound, deuterated derivative, salt, or composition for use according to any one of Embodiments 159-167, wherein the one or more additional therapeutic agent(s) comprise(s) at least one compound selected from FDL-176, PTI-808, GLPG1837/ABBV-974, GLPG2451/ABBV-2451, QBW251 (Icenticaftor), GLPG3067/ABBV-3067 (Navocaftor), and ABBV-191.
169. The compound, deuterated derivative, salt, or composition for use according to any one of Embodiments 159-168, wherein the one or more additional therapeutic agent(s) comprise(s) a CFTR amplifier.
170. The compound, deuterated derivative, salt, or composition for use according to any one of Embodiments 159-169, wherein the one or more additional therapeutic agent(s) comprise(s) PTI-428.
171. The compound, deuterated derivative, salt, or composition for use according to any one of Embodiments 159-170, wherein the one or more additional therapeutic agent(s) comprise(s) a CFTR readthrough agent.
172. The compound, deuterated derivative, salt, or composition for use according to any one of Embodiments 159-171, wherein the one or more additional therapeutic agent(s) comprise(s) ELX-02.
173. The compound, deuterated derivative, salt, or composition for use according to any one of Embodiments 159-172, wherein the one or more additional therapeutic agent(s) comprise(s) a nucleic acid therapy.
174. The compound, deuterated derivative, salt, or composition for use according to any one of Embodiments 159-173, wherein the one or more additional therapeutic agent(s) comprise(s) at least one agent selected from MRT5005, Lunar-CF, and RCT223.
175. The compound, deuterated derivative, salt, or composition for use according to any one of Embodiments 159-174, wherein the one or more additional therapeutic agent(s) comprise(s) an ENaC inhibitor.
176. The compound, deuterated derivative, salt, or composition for use according to any one of Embodiments 159-175, wherein the one or more additional therapeutic agent(s) comprise(s) amiloride, ETD001, CF552, GS-9411, GS-5737, P-1037 (VX-371), P-1055 (VX-551), AZD5634, SPX-101, Ionis-ENaC-2.5 Rx, BI 1265162, and ARO-ENaC1001.
177. The compound, deuterated derivative, salt, or composition for use according to any one of Embodiments 159-176, wherein the one or more additional therapeutic agent(s) comprise(s) a TMEM16A modulator.
178. The compound, deuterated derivative, salt, or composition for use according to any one of Embodiments 159-177, wherein the one or more additional therapeutic agent(s) comprise(s) ETD002.
179. The compound, deuterated derivative, salt, or composition for use according to any one of Embodiments 159-178, wherein the one or more additional therapeutic agent(s) comprise(s) a GPR39 agonist.
180. The compound, deuterated derivative, salt, or composition for use according to any one of Embodiments 159-179, wherein the one or more additional therapeutic agent(s) comprise(s) DS-1039.
181. Use of Compound I or a deuterated derivative or pharmaceutically acceptable salt thereof, or a pharmaceutical composition according to any one of Embodiments 68-112, in the manufacture of a medicament for treating cystic fibrosis, wherein the medicament is formulated to administer about 0.1 mg to about 800 mg Compound I or an equivalent amount of a deuterated derivative or pharmaceutically acceptable salt thereof.
182. The use according to Embodiment 181, wherein the medicament is formulated to administer about 1 mg to about 400 mg of Compound I or an equivalent amount of a deuterated derivative or pharmaceutically acceptable salt thereof.
183. The use according to Embodiment 181 or Embodiment 182, wherein the medicament is formulated to administer about 5 mg to about 375 mg of Compound I or an equivalent amount of a deuterated derivative or pharmaceutically acceptable salt thereof.
184. The use according to any one of Embodiments 181-183, wherein the medicament is formulated to administer about 30 mg to about 300 mg of Compound I or an equivalent amount of a deuterated derivative or pharmaceutically acceptable salt thereof.
185. The use according to any one of Embodiments 181-184, wherein the medicament is formulated to administer about 50 mg to about 200 mg of Compound I or an equivalent amount of a deuterated derivative or pharmaceutically acceptable salt thereof.
186. The use according to any one of Embodiments 181-185, wherein the medicament is formulated to administer about 50 mg to about 175 mg of Compound I or an equivalent amount of a deuterated derivative or pharmaceutically acceptable salt thereof.
187. The use according to any one of Embodiments 181-183, wherein the medicament is formulated to administer about 10 mg to about 200 mg of Compound I or an equivalent amount of a deuterated derivative or pharmaceutically acceptable salt thereof.
188. The use according to any one of Embodiments 181-183, or 187, wherein the medicament is formulated to administer about 20 mg to about 100 mg of Compound I or an equivalent amount of a deuterated derivative or pharmaceutically acceptable salt thereof.
189. The use according to any one of Embodiments 181-183, 187, or 188, wherein the medicament is formulated to administer about 30 mg to about 60 mg of Compound I or an equivalent amount of a deuterated derivative or pharmaceutically acceptable salt thereof.
190. The use according to Embodiment 181 or Embodiment 182, wherein the medicament is formulated to administer about 1 mg to about 50 mg of Compound I or an equivalent amount of a deuterated derivative or pharmaceutically acceptable salt thereof.
191. The use according to any one of Embodiments 181, 182, or 190, wherein the medicament is formulated to administer about 2 mg to about 40 mg of Compound I or an equivalent amount of a deuterated derivative or pharmaceutically acceptable salt thereof.
192. The use according to any one of Embodiments 181-183, or 190, wherein the medicament is formulated to administer about 5 mg to about 50 mg of Compound I or an equivalent amount of a deuterated derivative or pharmaceutically acceptable salt thereof.
193. The use according to any one of Embodiments 181-183, or 190-192, wherein the medicament is formulated to administer about 5 mg to about 20 mg of Compound I or an equivalent amount of a deuterated derivative or pharmaceutically acceptable salt thereof.
194. The use according to any one of Embodiments 181-183, or 190-193, wherein the medicament is formulated to administer about 5 mg to about 10 mg of Compound I or an equivalent amount of a deuterated derivative or pharmaceutically acceptable salt thereof.
195. The use according to any one of Embodiments 181, 182, or 190, wherein the medicament is formulated to administer about 1 mg to about 30 mg of Compound I or an equivalent amount of a deuterated derivative or pharmaceutically acceptable salt thereof.
196. The use according to any one of Embodiments 181-184, wherein the medicament is formulated to administer about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg of Compound I or an equivalent amount of a deuterated derivative or pharmaceutically acceptable salt thereof.
197. The use according to any one of Embodiments 181-184, wherein the medicament is formulated to administer about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, or about 210 mg of Compound I or an equivalent amount of a deuterated derivative or pharmaceutically acceptable salt thereof.
198. The use according to any one of Embodiments 181-184, wherein the medicament is formulated to administer about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, or about 300 mg of Compound I or an equivalent amount of a deuterated derivative or pharmaceutically acceptable salt thereof.
199. The use according to any one of Embodiments 181, 182, or 190, wherein the medicament is formulated to administer about 1 mg, about 2 mg, about 3 mg, about 4 mg, about 5 mg, about 7.5 mg, about 10 mg, about 15 mg, about 20 mg, about 25 mg, about 30 mg, about 35 mg, about 40 mg, about 45 mg, or about 50 mg of Compound I or an equivalent amount of a deuterated derivative or pharmaceutically acceptable salt thereof.
200. The use according to Embodiment 181 or Embodiment 182, wherein the medicament is formulated to administer about 5 mg, about 7.5 mg, about 30 mg, about 35 mg, about 70 mg, about 75 mg, about 150 mg, about 375 mg, about 385 mg, or about 400 mg of Compound I or an equivalent amount of a deuterated derivative or pharmaceutically acceptable salt thereof daily.
201. The use according to Embodiment 181 or Embodiment 182, wherein the medicament is formulated to administer about 5 mg, about 7.3 mg, about 34 mg, about 73 mg, about 100 mg, about 150 mg, about 220 mg, or about 390 mg of Compound I or an equivalent amount of a deuterated derivative or pharmaceutically acceptable salt thereof daily.
202. The use according to any one of Embodiments 181-184, or 187-189, wherein the medicament is formulated to administer about 30 mg, about 31 mg, about 32 mg, about 33 mg, about 34 mg, about 35 mg, about 36 mg, about 37 mg, about 38 mg, about 39 mg, about 40 mg, or about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, or about 60 mg of Compound I or an equivalent amount of a deuterated derivative or pharmaceutically acceptable salt thereof.
203. The use according to any one of Embodiments 181-202, wherein the medicament is formulated to be administered as a single dose, once daily.
204. The use according to any one of Embodiments 181-202, wherein the medicament is formulated to be administered in two doses daily.
205. The use according to any one of Embodiments 181-204, wherein the medicament is formulated to be administered in combination with one or more additional therapeutic agent(s).
206. The use according to Embodiment 205, wherein the one or more additional therapeutic agent(s) comprise(s) a compound with CFTR-modulating activity or a deuterated derivative or pharmaceutically acceptable salt thereof.
207. The use according to Embodiment 205 or Embodiment 206, wherein the one or more additional therapeutic agent(s) comprise(s) a CFTR corrector.
208. The use according to any one of Embodiments 205-207, wherein the one or more additional therapeutic agent(s) comprise(s) a compound selected from:
(a) Compound II,
(b) Compound IV,
(c) Compound V,
(d) Compound VI,
(e) Compound VII,
(f) Compound VIII,
(g) Compound IX, and
(h) Compound X, and deuterated derivatives and pharmaceutically acceptable salts thereof.
209. The use according to any one of Embodiments 205-208, wherein the one or more additional therapeutic agent(s) comprise(s) at least one compound selected from PTI-428, ABBV-2222, ABBV-2851, GLPG2737, ABBV-3221, ABBV-3748, ABBV-3903, ABBV-119, ABBV-2851, FDL-169, ARN5562, ARN21586, ARN22081, ARN22652, ARN23765, ARN23766, and PTI-801.
210. The use according to any one of Embodiments 205-209, wherein the one or more additional therapeutic agent(s) comprise(s) a CFTR potentiator enhancer.
211. The use according to any one of Embodiments 205-210, wherein the one or more additional therapeutic agent(s) comprise(s) ASP-11.
212. The use according to any one of Embodiments 205-211, wherein the one or more additional therapeutic agent(s) comprise(s) a CFTR potentiator.
213. The use according to any one of Embodiments 205-212 wherein the one or more additional therapeutic agent(s) comprise(s) a compound selected from Compound III, Compound III-d, and deuterated derivatives and pharmaceutically acceptable salts thereof.
214. The use according to any one of Embodiments 205-213, wherein the one or more additional therapeutic agent(s) comprise(s) at least one compound selected from FDL-176, PTI-808, GLPG1837/ABBV-974, GLPG2451/ABBV-2451, QBW251 (Icenticaftor), GLPG3067/ABBV-3067 (Navocaftor), and ABBV-191.
215. The use according to any one of Embodiments 205-214, wherein the one or more additional therapeutic agent(s) comprise(s) a CFTR amplifier.
216. The use according to any one of Embodiments 205-215, wherein the one or more additional therapeutic agent(s) comprise(s) PTI-428.
217. The use according to any one of Embodiments 205-216, wherein the one or more additional therapeutic agent(s) comprise(s) a CFTR readthrough agent.
218. The use according to any one of Embodiments 205-217, wherein the one or more additional therapeutic agent(s) comprise(s) ELX-02.
219. The use according to any one of Embodiments 205-218, wherein the one or more additional therapeutic agent(s) comprise(s) a nucleic acid therapy.
220. The use according to any one of Embodiments 205-219, wherein the one or more additional therapeutic agent(s) comprise(s) at least one agent selected from MRT5005, Lunar-CF, and RCT223.
221. The use according to any one of Embodiments 205-220, wherein the one or more additional therapeutic agent(s) comprise(s) an ENaC inhibitor.
222. The use according to any one of Embodiments 205-221, wherein the one or more additional therapeutic agent(s) comprise(s) amiloride, ETD001, CF552, GS-9411, GS-5737, P-1037 (VX-371), P-1055 (VX-551), AZD5634, SPX-101, Ionis-ENaC-2.5 Rx, BI 1265162, or ARO-ENaC1001.
223. The use according to any one of Embodiments 205-222, wherein the one or more additional therapeutic agent(s) comprise(s) a TMEM16A modulator.
224. The use according to any one of Embodiments 205-223, wherein the one or more additional therapeutic agent(s) comprise(s) ETD002.
225. The use according to any one of Embodiments 205-224, wherein the one or more additional therapeutic agent(s) comprise(s) a GPR39 agonist.
226. The use according to any one of Embodiments 205-225, wherein the one or more additional therapeutic agent(s) comprise(s) DS-1039.

### Examples

### Abbreviations

AE: adverse event
AUC: area under the concentration versus time curve
AUC_{0-∞}: AUC from the time of dosing extrapolated to infinity
AUC₀₋₂₄h: AUC from the time of dosing to 24 hours
AUC₀₋ₜₗₐₛₜ: AUC from the time of dosing to the last measurable concentration
BA: bioavailability
Boc anhydride ((Boc) 20): Di-tert-butyl 192yridine192the
Boc: tert-Butyloxycarbonyl
Cmax: maximum observed concentration
DCE: 1,2-Dichloroethane
DDI: drug-drug interaction
DIAD: Diisopropyl azodicarboxylate
193yri: DIPEA or N,N-Diisopropylethylamine
DMF: N,N-Dimethylformamide
DMSO: Dimethyl sulfoxide
DMSO-d6: Dimethyl sulfoxide-d6
EtOAc: Ethyl acetate
ESI-MS: Electrospray ionization mass spectrometry
FIH: first-in-human (study)
HATU: N-[(Dimethylamino)-1H-1,2,3-triazolo-[4,5-b]193yridine-1-ylmethylene]-N-methylmethanaminium hexafluorophosphate N-oxide
HBE: human bronchial epithelial (cells)
HPLC: High performance liquid chromatography
LC: Liquid chromatography
MS: Mass spectrometry
MTBE:
n: number of subjects
NMR: Nuclear magnetic resonance
NOAEL: no-observed-adverse-effect level
PD: pharmacodynamic, pharmacodynamics
Pd/C: Palladium on carbon
PK: pharmacokinetic, pharmacokinetics
q12h: every 12 hours
qd: once daily
RT or rt: Room temperature
TBD: to be determined
TFA: Trifluoroacetic acid
UPLC: Ultra Performance Liquid Chromatography

### General UPLC-MS/HPLC-MS/GC Analytical Method:

LC Method D: Analytical reverse phase UPLC-MS using an Acquity UPLC-MS BEH C₁₈ column (50× 2.1 mm, 1.7 μm particle size) made by Waters (pn: 186002350), and a dual gradient run from 1% to 99% mobile phase B over 5.0 minutes. Mobile phase A=water (+0.05% trifluoroacetic acid). Mobile phase B=acetonitrile (+0.035% trifluoroacetic acid). Flow rate=1.2 mL/min, injection volume=1.5 μL, and column temperature=60° C.

### General X-Ray Powder Diffraction (XRPD) Method

The X-ray powder diffraction (XRPD) pattern was recorded at room temperature in continuous mode using a PANalytical Empyrean X-ray Diffract meter (Almelo, The Netherlands). The X-ray was generated using Cu tube operated at 45 kV and 40 mA. Pixel 1d detector was used with anti-scatter slit P8. The Divergence optics was Bragg Brentano High Definition (BBHD) with a 10 mm mask, ⅛ divergence slit, and ½ anti-scatter slit. The continuous scan mode utilized a 0.0131 degree step size and count time of 13.77 seconds per step, integrated over the range from 4 to 40 degrees two-theta. The powder sample was placed on an indented area within a zero background holder and flattened with a glass slide.

General Differential Scanning calorimetry (DSC) Method

Unless provided otherwise in the following Examples, the melting point or glass transition point of the material was measured using a Mettler Toledo TGA/DSC 3+ STARe System.

### Example 1: Preparation of (6R)-17-amino-12,12-dimethyl-6,15-bis(trifluoromethyl)-19-oxa-3,4,13,18-tetrazatricyclo[12.3.1.12,5]nonadeca-1 (18),2,4,14,16-pentaen-6-ol (Compound I)

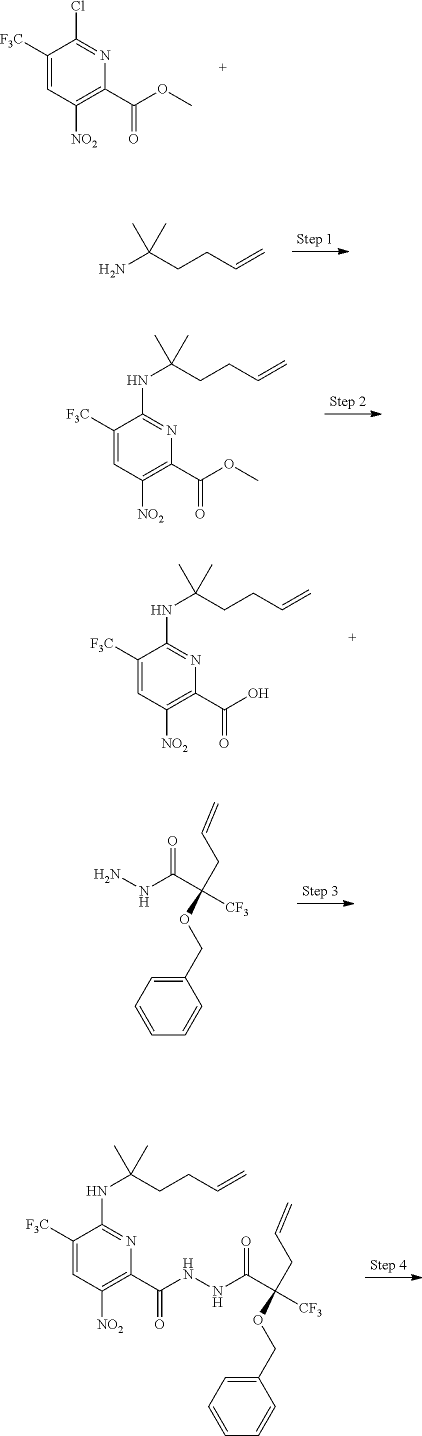

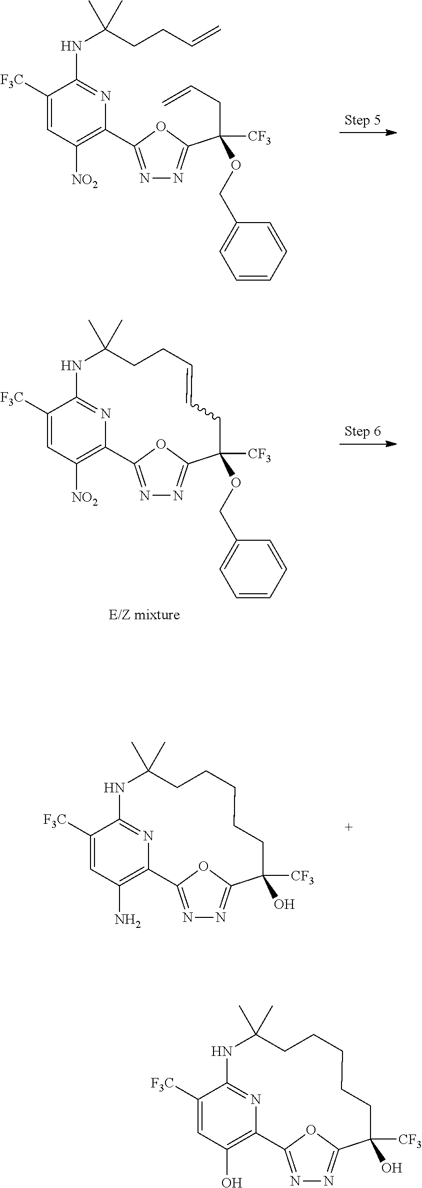 [SMILES: C=CCCC(C)(C)Nc1nc(-c2nnc([C@@](CC=C)(OCc3ccccc3)C(F)(F)F)o2)c([N+](=O)[O-])cc1C(F)(F)F ; CC1(C)CCC=CC[C@](OCc2ccccc2)(C(F)(F)F)C2=NN=C(O2)c2nc(c(C(F)(F)F)cc2[N+](=O)[O-])N1 ; CC1(C)CCCCC[C@](O)(C(F)(F)F)C2=NN=C(O2)c2nc(c(C(F)(F)F)cc2N)N1 ; CC1(C)CCCCC[C@](O)(C(F)(F)F)C2=NN=C(O2)c2nc(c(C(F)(F)F)cc2O)N1]

### Step 1: Methyl 6-(1,1-dimethylpent-4-enylamino)-3-nitro-5-(trifluoromethyl)pyridine-2-carboxylate

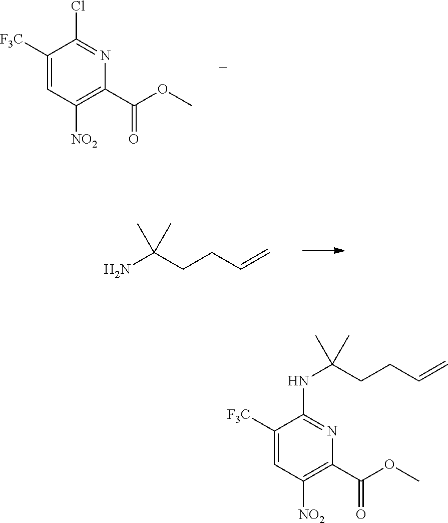

2-Methylhex-5-en-2-amine (hydrochloride salt) (69.4 g, 463.7 mmol) was suspended in acetonitrile (960 mL) and treated with 195yri (220 mL, 1.263 mol). To the formed brown solution was added methyl 6-chloro-3-nitro-5-(trifluoromethyl)pyridine-2-carboxylate (120 g, 421.7 mmol) in one portion. The orange solution was slowly heated to 65° C. over 2.5 h (Note: reaction shows exotherm on heating). The deep orange solution was evaporated at 40° C. and to the residue was added with MTBE (1 L) and water (1 L) and the layers were separated. The deep orange organic phase was washed with a 1:1 solution of saturated aqueous NH₄Cl/water mixture (2×600 mL), once with brine (400 mL) and the organic phase was dried, filtered and evaporated to give methyl 6-(1,1-dimethylpent-4-enylamino)-3-nitro-5-(trifluoromethyl)pyridine-2-carboxylate (152.7 g, 100%). ESI-MS m/z calc. 361.12494, found 362.0 (M+1)⁺; Retention time: 3.02 minutes (LC Method D).

### Step 2:6-(1,1-Dimethylpent-4-enylamino)-3-nitro-5-(trifluoromethyl)pyridine-2-carboxylic acid

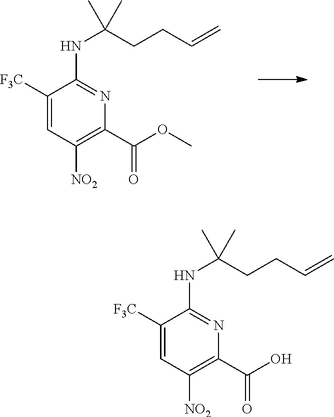 [SMILES: C=CCCC(C)(C)Nc1nc(C(=O)OC)c([N+](=O)[O-])cc1C(F)(F)F ; C=CCCC(C)(C)Nc1nc(C(=O)O)c([N+](=O)[O-])cc1C(F)(F)F]

Methyl 6-(1,1-dimethylpent-4-enylamino)-3-nitro-5-(trifluoromethyl)pyridine-2-carboxylate (152.4 g, 421.7 mmol) was dissolved in methanol (750 mL) and treated with NaOH (750 mL of 2 M, 1.500 mol) under stirring (added all at once giving a slight exotherm from 30° C. to 40° C.). The solution was stirred at room temperate for 18 h. The deep red solution was concentrated under reduced pressure at 42° C. and the resulting orange red solution was treated with toluene (1 L). The emulsion was stirred in an ice bath and acidified to pH=1 by addition of HCl (260 mL of 6 M, 1.560 mol) keeping the internal temperature below 15° C. The phases were separated and the organic phase was washed twice with water (2×500 mL) and once with brine (400 mL). The organic phase was dried over MgSO₄, filtered, evaporated and dried under vacuum to give 137 g of a deep orange mass of solid. This material was evaporated from acetonitrile (˜1 L, to remove residual toluene) and dissolved in acetonitrile (600 mL) and warmed to ˜60° C. To the deep red hot solution was added N-cyclohexylcyclohexanamine (79 mL, 396.5 mmol) under stirring (exotherm noted from 60° C. to 70° C.) and the hot solution was seeded. The material became a solid mass at an internal temperature of ˜ 60° C., which could be stirred magnetically after breaking up. The thick suspension was stirred in the cooling hot water bath overnight and then in an ice bath for 3 h. The solid was collected by filtration, washed with cold acetonitrile until the filtrate was colorless and dried over the weekend to give 6-(1,1-dimethylpent-4-enylamino)-3-nitro-5-(trifluoromethyl)pyridine-2-carboxylic acid (dicyclohexylamine salt) (172 g, 77%) as a yellow solid. This salt was suspended in MTBE (1 L) and treated with citric acid (1.2 L of 1 M, 1.200 mol). The mixture was stirred and the phases were separated. The organic phase was washed twice more with 1 M citric acid (2×400 mL) and 4 times with 0.5M KHSO₄ (4×400 mL). The organic phase was then washed once with brine (200 mL), dried, filtered and evaporated to give 6-(1,1-dimethylpent-4-enylamino)-3-nitro-5-(trifluoromethyl)pyridine-2-carboxylic acid (113.4 g, 77%) as a yellow orange oil, which crystallized upon standing. ¹H NMR (400 MHZ, DMSO-d6) δ 14.21 (s, 1H), 8.46 (s, 1H), 6.20-6.00 (m, 1H), 5.82-5.57 (m, 1H), 5.13-4.74 (m, 2H), 1.97 (d, J=2.9 Hz, 4H), 1.45 (s, 6H) ppm. ESI-MS m/z calc. 347.10928, found 348.0 (M+1)⁺; Retention time: 2.49 minutes (LC Method D).

### Step 3: N′-[(2R)-2-Benzyloxy-2-(trifluoromethyl) pent-4-enoyl]-6-(1,1-dimethylpent-4-enylamino)-3-nitro-5-(trifluoromethyl)pyridine-2-carbohydrazide

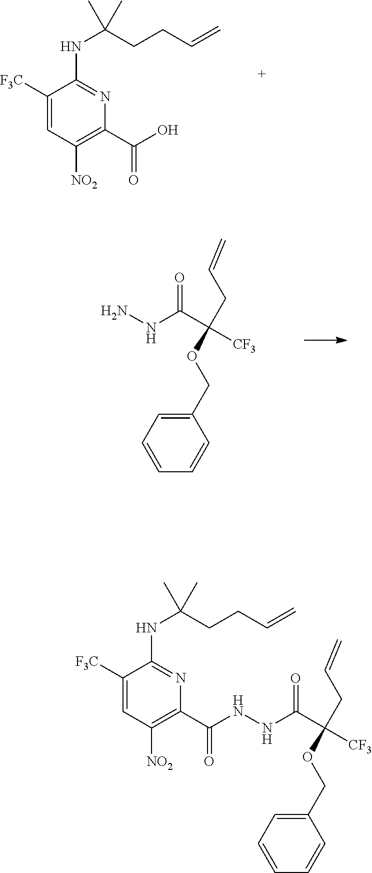 [SMILES: C=CCCC(C)(C)Nc1nc(C(=O)O)c([N+](=O)[O-])cc1C(F)(F)F ; C=CC[C@@](OCc1ccccc1)(C(=O)NN)C(F)(F)F ; C=CCCC(C)(C)Nc1nc(C(=O)NNC(=O)[C@@](CC=C)(OCc2ccccc2)C(F)(F)F)c([N+](=O)[O-])cc1C(F)(F)F]

6-(1,1-Dimethylpent-4-enylamino)-3-nitro-5-(trifluoromethyl)pyridine-2-carboxylic acid (100 g, 285.1 mmol) and (2R)-2-benzyloxy-2-(trifluoromethyl) pent-4-enehydrazide (86.3 g, 299.4 mmol) were dissolved in DMF (600 mL) and cooled in an ice bath. At an internal temperature of 3.1° C., HATU (114 g, 299.8 mmol) was added in one portion (no exotherm observed). Then, 196yri (100 mL, 574.1 mmol) was slowly added over 0.5 h (exothermic) keeping the internal temperate between 3 and 10° C. After the addition, the ice bath was removed and the reaction was stirred for another 0.5 h allowing it to warm to room temperature. The orange solution was added to a stirred solution of ice and water (3 L) and MTBE (1 L). The mixture was stirred for 10 minutes and the phases were separated. The organic phase was washed twice with water (2×1 L), 0.2 M KHSO₄ (3×1 L) and once with brine (250 mL). The organic phase was dried, filtered and evaporated to give as an orange mass, N′-[(2R)-2-benzyloxy-2-(trifluoromethyl) pent-4-enoyl]-6-(1,1-dimethylpent-4-enylamino)-3-nitro-5-(trifluoromethyl)pyridine-2-carbohydrazide (181 g, quantitative yield). ESI-MS m/z calc. 617.2073, found 618.0 (M+1)⁺; Retention time: 3.25 minutes (LC Method D). This material was used directly in the next step.

### Step 4:6-[5-[(1R)-1-Benzyloxy-1-(trifluoromethyl) but-3-enyl]-1,3,4-oxadiazol-2-yl]-N-(1,1-dimethylpent-4-enyl)-5-nitro-3-(trifluoromethyl)pyridine-2-amine

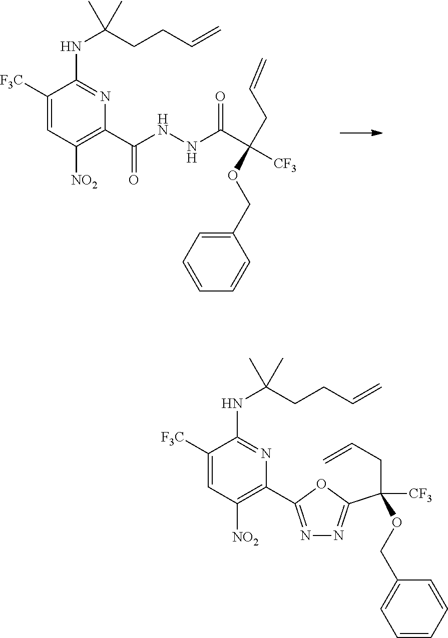 [SMILES: C=CCCC(C)(C)Nc1nc(C(=O)NNC(=O)[C@@](CC=C)(OCc2ccccc2)C(F)(F)F)c([N+](=O)[O-])cc1C(F)(F)F ; C=CCCC(C)(C)Nc1nc(-c2nnc([C@@](CC=C)(OCc3ccccc3)C(F)(F)F)o2)c([N+](=O)[O-])cc1C(F)(F)F]

N′-[(2R)-2-Benzyloxy-2-(trifluoromethyl) pent-4-enoyl]-6-(1,1-dimethylpent-4-enylamino)-3-nitro-5-(trifluoromethyl)pyridine-2-carbohydrazide (176.1 g, 285.2 mmol) was dissolved in acetonitrile (1.4 L) and heated to 55° C. The yellow orange solution was treated with 197yri (124 mL, 711.9 mmol) followed by portion-wise addition of tosyl chloride (54.4 g, 285.3 mmol) over 15 min (exothermic, internal temperature kept between 55° C. and 60° C. by removing the heating mantel and slow addition) and the reaction was stirred at 55-60° C. for 45 min. The reaction solution was concentrated under reduced pressure at 40° C. and the residue was extracted with MTBE/heptane 1:1 (1.4 L) and water (1.4 L). The organic phase was washed once more with water (1.5 L), twice with 0.2M KHSO₄ (2×1 L) and once with brine (0.5 L). The organic phase was dried, filtered and evaporated to give 172 g of an orange oil which was dissolved in 100 mL of toluene and 300 mL of heptane. The solution was loaded onto a 3 kg silica column (column volume=4800 mL, flow rate=900 mL/min). Eluted with 100% hexanes for 1 min, then programmed an initial gradient of 0% to 10% ethyl acetate in hexanes over 106 min (2 column volumes). The product started eluting at ˜4% ethyl acetate, so 4.3% ethyl acetate was held isocratically until the product finished eluting to give 6-[5-[(1R)-1-benzyloxy-1-(trifluoromethyl) but-3-enyl]-1,3,4-oxadiazol-2-yl]-N-(1,1-dimethylpent-4-enyl)-5-nitro-3-(trifluoromethyl) 197yridine-2-amine (139.1 g, 80%). ¹H NMR (400 MHZ, Chloroform-d) § 8.51 (s, 1H), 7.40-7.27 (m, 5H), 6.03-5.87 (m, 1H), 5.80-5.66 (m, 1H), 5.58 (s, 1H), 5.31-5.16 (m, 2H), 5.03-4.95 (m, 1H), 4.95-4.89 (m, 1H), 4.81 (d, J=10.5 Hz, 1H), 4.64 (d, J=10.5 Hz, 1H), 3.28-3.13 (m, 2H), 2.08-1.99 (m, 2H), 1.99-1.89 (m, 2H), 1.47 (s, 6H) ppm. ESI-MS m/z calc. 599.1967, found 600.0 (M+1)⁺; Retention time: 3.71 minutes (LC Method D).

### Step 5: (6R)-6-Benzyloxy-12,12-dimethyl-17-nitro-6,15-bis(trifluoromethyl)-19-oxa-3,4,13,18-tetrazatricyclo[12.3.1.12,5]nonadeca-1 (18),2,4,8,14,16-hexaene (E/Z mixture)

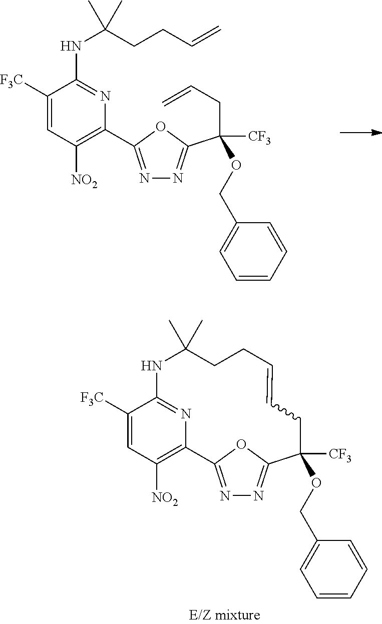 [SMILES: C=CCCC(C)(C)Nc1nc(-c2nnc([C@@](CC=C)(OCc3ccccc3)C(F)(F)F)o2)c([N+](=O)[O-])cc1C(F)(F)F ; CC1(C)CCC=CC[C@](OCc2ccccc2)(C(F)(F)F)C2=NN=C(O2)c2nc(c(C(F)(F)F)cc2[N+](=O)[O-])N1]

This reaction was run in three, 46.3 g batches in parallel, each in a 12 L, 3-neck round bottom flask. The experimental below describes one of these batches.

Attached a sparging tube, reflux condenser with gas bubbler & overhead stirrer to a 12 L vessel placed in heat blanket. Dissolved 6-[5-[(1R)-1-benzyloxy-1-(trifluoromethyl) but-3-enyl]-1,3,4-oxadiazol-2-yl]-N-(1,1-dimethylpent-4-enyl)-5-nitro-3-(trifluoromethyl) 198yridine-2-amine (46.3 g, 76.22 mmol) in DCE (8.23 L). Sparged the system with a heavy stream of nitrogen gas. Set the heat blanket for 50° C. When the vessel reached 53° C., added dichloro[1,3-bis(2,4,6-trimethylphenyl)-2-imidazolidinylidene][[5-[(dimethylamino)sulfonyl]-2-(1-methylethoxy-O)phenyl]methylene-C]ruthenium (II) (Zhan catalyst-1B, 11.2 g, 15.26 mmol) all at once. Rinsed the catalyst container with DCE and added the rinse to the reaction. On completion of catalyst addition, increased the blanket temperature to 73° C. Once internal temperature reached 72° C., continued stirring for 2 h 28 min then decreased the heat blanket temperature to 45° C. After 2 h 27 min, internal temperature reached 50° C. After 15 min, added solid 2-sulfanylpyridine-3-carboxylic acid (12 g, 77.33 mmol) and triethylamine (11 mL, 78.92 mmol). Stirred for 12 h then allowed the mixture to cool to room temperature. Added 100 g of SiO₂ and 10 g of activated carbon (20-40 mesh, granular) to the reaction. Stirred for 1 h then filtered over Celite and evaporated the filtrate giving the crude product mixture. Combined the material from all three parallel reactions to give 71.2 g of crude product mixture. This material was purified on two separate 3 kg silica gel columns using a gradient from 100% hexanes to 10% ethyl acetate in hexanes over 110 min followed by a gradient from 10% ethyl acetate in hexanes to 100% ethyl acetate over 10 minutes. After combining the two separate purified batches, obtained (6R)-6-benzyloxy-12,12-dimethyl-17-nitro-6,15-bis(trifluoromethyl)-19-oxa-3,4,13,18-tetrazatricyclo[12.3.1.12,5]nonadeca-1 (18),2,4,8,14,16-hexaene (E/Z mixture) (51.88 g, 40%). ¹H NMR (400 MHZ, DMSO-d6) δ 8.55 (d, J=0.8 Hz, 1H), 7.49-7.21 (m, 5H), 6.58 (s, 1H), 5.79 (dt, J=13.7, 6.5 Hz, 1H), 5.58 (ddd, J=15.0, 8.8, 5.6 Hz, 1H), 4.83 (d, J=11.1 Hz, 1H), 4.55 (d, J=11.1 Hz, 1H), 3.13 (dd, J=14.2, 5.4 Hz, 1H), 2.77 (dd, J=14.3, 8.8 Hz, 1H), 2.38-2.24 (m, 1H), 2.14-1.93 (m, 3H), 1.58-1.32 (m, 6H) ppm. ESI-MS m/z calc. 571.1654, found 572.1 (M+1)⁺; Retention times: 3.46 minutes and 3.49 minutes (LC Method D). Product formed as a 3:1 mixture of double bond isomers.

### Step 6: (6R)-17-Amino-12,12-dimethyl-6,15-bis(trifluoromethyl)-19-oxa-3,4,13,18-tetrazatricyclo[12.3.1.12,5]nonadeca-1 (18),2,4,14,16-pentaen-6-ol (Compound I) and (6R)-12,12-dimethyl-6,15-bis(trifluoromethyl)-19-oxa-3,4,13,18-tetrazatricyclo[12.3.1.12,5]nonadeca-1 (18),2,4,14,16-pentaene-6,17-diol

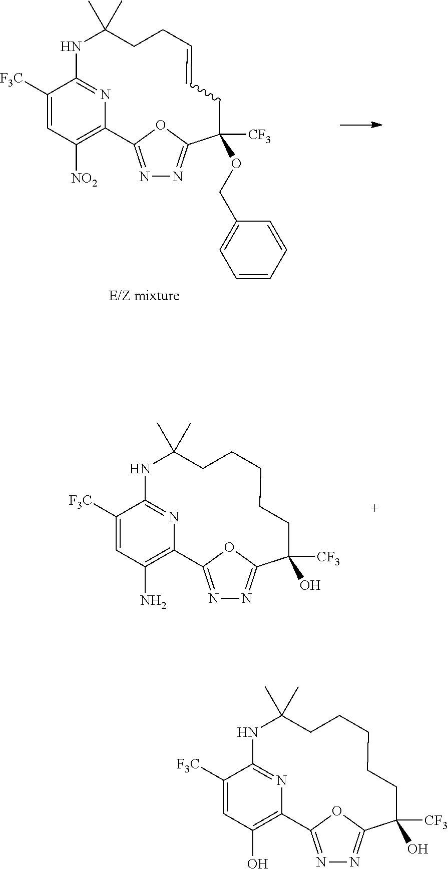 [SMILES: CC1(C)CCC=CC[C@](OCc2ccccc2)(C(F)(F)F)C2=NN=C(O2)c2nc(c(C(F)(F)F)cc2[N+](=O)[O-])N1 ; CC1(C)CCCCC[C@](O)(C(F)(F)F)C2=NN=C(O2)c2nc(c(C(F)(F)F)cc2N)N1 ; CC1(C)CCCCC[C@](O)(C(F)(F)F)C2=NN=C(O2)c2nc(c(C(F)(F)F)cc2O)N1]

(6R)-6-Benzyloxy-12,12-dimethyl-17-nitro-6,15-bis(trifluoromethyl)-19-oxa-3,4,13,18-tetrazatricyclo[12.3.1.12,5]nonadeca-1 (18),2,4,8,14,16-hexaene (E/Z mixture) (50.8 g, 88.89 mmol) was dissolved in 250 mL of ethanol and partially concentrated by rotary evaporation with 28° C. water bath to remove any residual solvents then dissolved in further ethanol (720 mL) in a 5 L flask. Degassed the solution using 5 cycles of house vacuum with nitrogen gas backfill. Added dihydroxypalladium (15.2 g of 10% w/w, 10.824 mmol) to the substrate solution under nitrogen. Repeated house vacuum with hydrogen backfill for 6 cycles to replace nitrogen atmosphere with hydrogen. Finally kept the vessel under 1 atmosphere of hydrogen using a balloon. Stirred this mixture vigorously with a magnetic stirrer overnight then removed the hydrogen balloon. Filtered the mixture through 70 g of Celite on a medium-fritted funnel. Concentrated the green filtrate solution by rotary evaporation with a 28° C. water bath. Obtained 42.65 g of crude product as a yellow solid of which 41.5 g was purified by reverse-phase chromatography (dissolved in 125 mL of methanol and 2.55 mL DMF (2% DMF/methanol solution) and loaded onto a 3.8 kg C₁₈ column (column volume=3.3 L, flow rate=375 mL/min). Programmed an initial gradient of 40% to 70% acetonitrile in water over 176 minutes (20 column volumes), then brought the eluent to 100% acetonitrile over the following ˜ 20 min). Mixed and pure fractions were isolated from the column. Pure fractions were concentrated to give as a yellow solid, (6R)-17-amino-12,12-dimethyl-6,15-bis(trifluoromethyl)-19-oxa-3,4,13,18-tetrazatricyclo[12.3.1.12,5]nonadeca-1 (18),2,4,14,16-pentaen-6-ol (28.17 g, 70%). This material was combined with several smaller batches (80 mg, 340 mg, 360 mg, 1.46 g and 1.63 g) made by similar methods as a solution in acetonitrile which was then concentrated to give a yellow solid. This solid was dissolved in dichloromethane and heptane was added then the solution was concentrated under vacuum in the dark at 40° C. overnight, which gave 31.95 g of (6R)-17-amino-12,12-dimethyl-6,15-bis(trifluoromethyl)-19-oxa-3,4,13,18-tetrazatricyclo[12.3.1.12,5]nonadeca-1 (18),2,4,14,16-pentaen-6-ol (Compound I). ¹H NMR (400 MHZ, DMSO-d6) δ 7.61 (s, 1H), 7.59 (s, 1H), 5.96 (s, 2H), 4.64 (s, 1H), 2.90-2.71 (m, 1H), 2.30-2.15 (m, 1H), 2.15-1.98 (m, 1H), 1.91-1.74 (m, 1H), 1.73-1.57 (m, 1H), 1.56-1.38 (m, 5H), 1.36 (s, 3H), 1.31 (s, 3H). ESI-MS m/z calc. 453.15994, found 454.2 (M+1)⁺; Retention time: 3.03 minutes (LC Method D).

One mixed fraction from the reverse-phase purification described above contained an impurity showing a mass one unit greater than the intended product described above. This fraction was concentrated and the residue was dissolved in 3.6 mL of methanol, then purified by reverse-phase prep HPLC through a C₁₈ column using a gradient from 1-99% acetonitrile in water (+HCl modifier) giving as a yellow solid, (6R)-12,12-dimethyl-6,15-bis(trifluoromethyl)-19-oxa-3,4,13,18-tetrazatricyclo[12.3.1.12,5]nonadeca-1 (18),2,4,14,16-pentaene-6,17-diol (105 mg, 0.003%). ¹H NMR (400 MHZ, DMSO-d6) δ 10.43 (s, 1H), 7.64 (s, 1H), 7.57 (s, 1H), 4.85 (s, 1H), 2.91-2.74 (m, 1H), 2.30-2.15 (m, 1H), 2.10-1.96 (m, 1H), 1.85-1.68 (m, 1H), 1.68-1.54 (m, 1H), 1.53-1.37 (m, 5H), 1.36 (s, 3H), 1.31 (s, 3H) ppm. ESI-MS m/z calc. 454.14395, found 455.2 (M+1)⁺; Retention time: 2.87 minutes (LC Method D).

### Step 7: Solid form characterization of crystalline Compound I Form A (neat)

### A. X-Ray Powder Diffraction

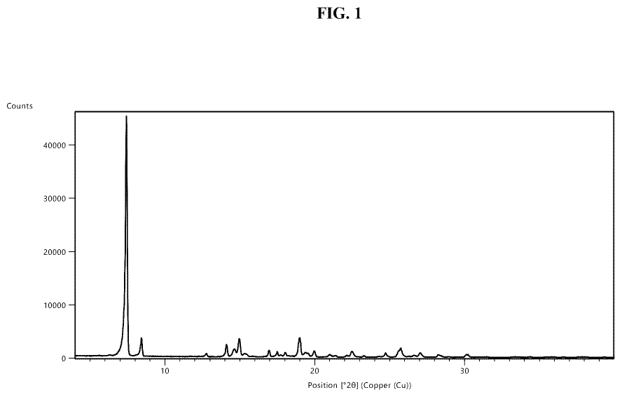

The XRPD diffractogram for crystalline Compound I Form A (neat) produced by Step 6 and recrystallized from EtOH was acquired using the General XRPD Method. The XRPD diffractogram for crystalline Compound I Form A (neat) is provided in FIG. 1, and the XRPD data are summarized below in Table 3.

TABLE 3
XRPD signals for crystalline Compound I Form A (neat)

| XRPD | Angle (degrees | |
|---|---|---|
| Peak No. | 2-Theta ± 0.2) | Intensity % |
| 1 | 7.4271 | 100 |
| 2 | 8.4377 | 7.56 |
| 3 | 14.1039 | 4.84 |
| 4 | 14.5744 | 2.53 |
| 5 | 14.962 | 7.2 |
| 6 | 16.9424 | 2.49 |
| 7 | 19.0503 | 5.71 |
| 8 | 19.9711 | 2.4 |
| 9 | 22.4778 | 2.31 |
| 10 | 25.5622 | 2.19 |
| 11 | 25.7502 | 3.64 |

B. Differential Scanning calorimetry Analysis

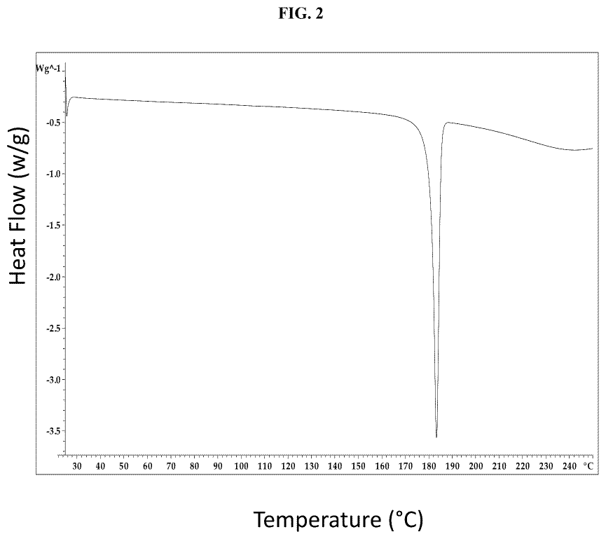

The DSC data were collected with a ramp of 10.00° C./min to 250.00° C. The DSC thermogram for crystalline Compound I Form A (neat) produced by Step 6 is provided in FIG. 2. The thermogram shows a Tm onset of 180.8° C., with a Tm peak at 183.18° C., 62.32 J/g.

### Example 2: Bioactivity Assay

### Ussing Chamber Assay of CFTR-Mediated Short-Circuit Currents

Ussing chamber experiments were performed using human bronchial epithelial (HBE) cells derived from CF subjects heterozygous for F508del and a minimal function CFTR mutation (F508del/MF-HBE) and cultured as previously described (Neuberger T, Burton B, Clark H, Van Goor F Methods Mol Biol 2011:741:39-54). After four days the apical media was removed, and the cells were grown at an air liquid interface for >14 days prior to use. This resulted in a monolayer of fully differentiated columnar cells that were ciliated, features that are characteristic of human bronchial airway epithelia.

To isolate the CFTR-mediated short-circuit (I_{SC}) current, F508del/MF-HBE grown on Costar® Snapwell™ cell culture inserts were mounted in an Ussing chamber and the transepithelial I_{SC} was measured under voltage-clamp recording conditions (V_{hold}=0 mV) at 37° C. The basolateral solution contained (in mM) 145 NaCl, 0.83 K₂HPO₄, 3.3 KH₂PO₄, 1.2 MgCl₂, 1.2 CaCl₂), 10 Glucose, 10 HEPES (pH adjusted to 7.4 with NaOH) and the apical solution contained (in mM) 145 NaGluconate, 1.2 MgCl₂, 1.2 CaCl₂), 10 glucose, 10 HEPES (pH adjusted to 7.4 with NaOH) and 30 μM amiloride to block the epithelial sodium channel. Forskolin (20 μM) was added to the apical surface to activate CFTR, followed by apical addition of a CFTR inhibitor cocktail consisting of BPO, GlyH-101 and CFTR inhibitor 172 (each at 20 μM final assay concentration) to specifically isolate CFTR currents. The CFTR-mediated I_{SC} (uA/cm2) for each condition was determined from the peak forskolin response to the steady-state current following inhibition.

### Identification of Potentiator Compounds

The activity of the CFTR potentiator compounds on the CFTR-mediated I_{SC} was determined in Ussing chamber studies as described above. The F508del/MF-HBE cell cultures were incubated with the potentiator compounds at a range of concentrations in combination with 10 μM (14S)-8-[3-(2-{dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1 (22),5,7,9,19 (23),20-hexaene-2,2,4-trione for 18-24 hours at 37° C. and in the presence of 20% human serum. The concentration of potentiator compounds and (14S)-8-[3-(2-{dispiro[2.0.2.1]heptan-7-yl}ethoxy)-1H-pyrazol-1-yl]-12,12-dimethyl-276-thia-3,9,11,18,23-pentaazatetracyclo[17.3.1.111,14.05,10]tetracosa-1 (22),5,7,9,19 (23),20-hexaene-2,2,4-trione used during the 18-24 hours incubations was kept constant throughout the Ussing chamber measurement of the CFTR-mediated I_{SC} to ensure compounds were present throughout the entire experiment. The efficacy and potency of the putative F508del potentiators was compared to that of the known Vertex potentiator, ivacaftor (N-[2,4-bis(1,1-dimethylethyl)-5-hydroxyphenyl]-1,4-dihydro-4-oxoquinoline-3-carboxamide).

Table 4 presents CFTR-modulating activity for Compound I using the assay described in this example (EC50: +++ is <500 nM).

TABLE 4
Assay Data for Compound I

| Molecule | EC₅₀ |
|---|---|
| Compound I | +++ |

### Example 3

### Study Rationale

In a first-in-human (FIH) clinical study of Compound I, the safety, tolerability, and pharmacokinetics (PK) of Compound I in healthy subjects was evaluated. Assessments of the relative bioavailability (BA) of Suspension 2 of Compound I compared to Suspension 1 of Compound I, food effect, and drug drug interactions (DDIs) between Compound I and itraconazole and midazolam were conducted.

### Study Design

This first-in-human (FIH) study of Compound I included three parts (Parts A, B, and C).

Approximately 154 healthy adult subjects were planned to be enrolled in planned cohorts. Approximately 76 subjects were planned to be enrolled in Part A (excluding optional Part A Cohorts), approximately 48 subjects in Part B (excluding optional Part B Cohorts), and approximately 30 subjects in Part C. There was no pre-specified ratio of males to females, but reasonable effort was made to enroll females of non-childbearing potential in all dosing cohorts. The final number of subjects in each analysis set is provided in Table 5.

TABLE 5
Analysis Population

| | Part A | Part B | Part C | Overall |
|---|---|---|---|---|
| All Subjects Set | 98 | 40 | 30 | 168 |
| Safety Set | 98 | 40 | 30 | 168 |

Notes:
All Subjects Set: all subjects in the study who were either randomized (for Part A Cohorts except A9 and Part B cohorts) or enrolled (for Cohort A9, C1, C2; defined as subject having data in the clinical database) or received at least 1 dose of study drug; Safety Set: all subjects who received at least 1 dose of study drug. Part A included Cohorts A1 through A8, A9, and A10. Part B included Cohorts B1 through B5. Part C included Cohorts C1 and C2.

### Parts A and B:

Six dose escalation cohorts were planned for Part A, and five dose escalation cohorts were planned for Part B. Additional cohorts (A7 and A8) in Part A were enrolled based on data from previous cohorts. In each dose escalation cohort, eight subjects were randomized 3:1 to receive Compound I or placebo as a suspension (Cohort A6 enrolled six subjects). Cohorts A1 through A6 were dosed under fasted conditions, and Cohorts A7 and A8 were dosed under fed conditions. Eight subjects were enrolled in food effect Cohort A9 to receive open label Compound I; dosing of Compound I in subsequent cohorts were under fed conditions. Twenty-eight subjects were enrolled in Cohort A10 to receive Compound I in a randomized, open-label, single-dose, 4-period, 4-sequence, crossover study to evaluate the relative bioavailability of two Compound I suspensions under fasted conditions and the effect of food (high-fat and low-fat) on the pharmacokinetics of one Compound I suspension. The decision to initiate successive cohorts and dose selection was based on safety and tolerability data from the preceding dose group(s), as well as available PK data from the preceding cohort.

### Part A:

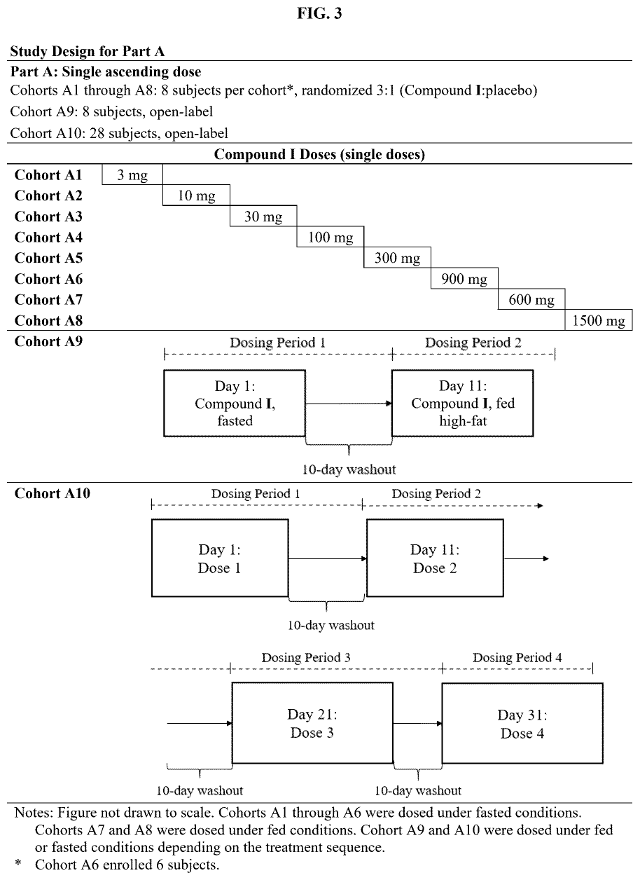

Part A (except Cohorts A9 and A10) was a randomized, double blind, placebo-controlled study evaluating single ascending doses of Compound I administered to healthy subjects (FIG. 3). The starting dose (Cohort A1) was 3 mg and the doses for Cohorts A2, A3, A4, A5, A6, A7, and A8 were 10 mg, 30 mg, 100 mg, 300 mg, 900 mg, 600 mg, and 1500 mg, respectively. Subject dosing was staggered in Part A (all cohorts excluding Cohorts A9 and A10) so that two sentinel subjects were dosed (one with Compound I and one with placebo) at least 48 hours before the remaining six subjects in each cohort. The remaining subjects in the cohort were dosed after safety data from the sentinel subjects was reviewed. Dose escalation occurred at increments of a maximum of half-log (3.3-fold) initially and the incremental increase occurred at no greater than 2-fold as exposures approached PK stopping criteria. Doses were repeated or modified based on emerging data.

Food effect was evaluated in an open label, single sequence Cohort A9 to evaluate the effect of food on the PK of Compound I based on emerging PK data during dose escalation. Cohort A9 received two doses of 200 mg each, with each dose administered ten days apart.

Cohort A10 was a randomized, open label, single-dose, four-period, four-sequence, crossover study to evaluate the relative BA of Suspension 2 of Compound I compared to Suspension 1 of Compound I, and the effect of food (high-fat and low-fat) on the PK of the Suspension 2 of Compound I, to inform formulation development. Cohort A10 was the only cohort in which Suspension 2 was administered. Cohort A10 received four doses of 200 mg each, with each dose administered ten days apart.

Subjects in Cohort A10 are equally randomized (1:1:1:1) on Day 1 to one of 4 treatment sequences using a balanced Latin Square design (Table 6). There are 4 treatments:
Treatment A: Compound I, Suspension 1, fasted
Treatment B: Compound I, Suspension 2, fasted
Treatment C: Compound I, Suspension 2, fed high-fat
Treatment D: Compound I, Suspension 2, fed low-fat

TABLE 6
Dosing Periods and Treatment Sequences for Cohort A10

| Treatment | | | | |
|---|---|---|---|---|
| Sequence | Dosing | Dosing | Dosing | Dosing |
| (n) | Period 1 | Period 2 | Period 3 | Period 4 |
| 1 (n = 7) | A | B | C | D |
| 2 (n = 7) | B | C | D | A |
| 3 (n = 7) | C | D | A | B |
| 4 (n = 7) | D | A | B | C |

n: number of subjects
Notes:
Treatment A: Compound I Suspension 1, fasted;
Treatment B: Compound I Suspension 2, fasted;
Treatment C: Compound I Suspension 2, fed high-fat;
Treatment D: Compound I Suspension 2, fed low-fat

### Part B:

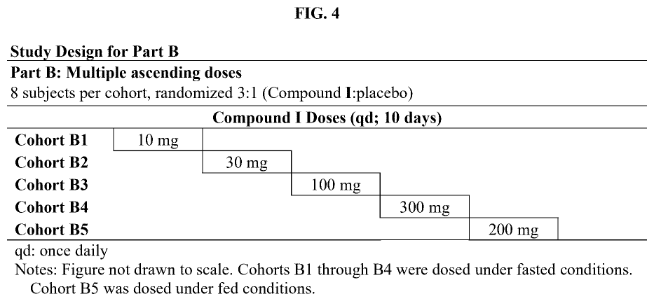

Part B was a randomized, double blind, and placebo-controlled study evaluating multiple ascending doses of Compound I administered to healthy subjects (FIG. 4). Part B was initiated while Part A was ongoing after review of safety, tolerability, and PK data. The total daily doses in Cohorts B1, B2, B3, B4, and B5 were 10 mg, 30 mg, 100 mg, 300 mg, and 200 mg, respectively. In Part B, sentinel dosing occurred in each cohort until the full range of anticipated exposures had been explored in Part A; it occurred in all Part B cohorts. The first two subjects were dosed (one with Compound I and one with placebo) at least five days before the remaining subjects in the cohort. The remaining subjects in the cohort were dosed after available safety data from the sentinel subjects had been reviewed. Dose escalation occurred at increments of a maximum of half-log (3.3-fold) initially and the incremental increase occurred at no greater than 2-fold as exposures approached PK stopping criteria. Subjects were dosed daily for ten days.

### CONCLUSIONS

Compound I was generally safe and well-tolerated across all cohorts.

### OTHER EMBODIMENTS

The foregoing discussion discloses and describes merely exemplary embodiments of this disclosure. One skilled in the art will readily recognize from such discussion and from the accompanying drawings and claims, that various changes, modifications, and variations can be made therein without departing from the spirit and scope of this disclosure as defined in the following claims.

1. A method of treating cystic fibrosis comprising administering to a patient in need thereof about 0.1 mg to about 800 mg of Compound I: 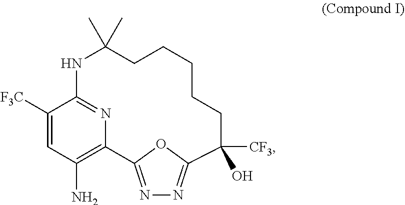 [SMILES: CC1(C)CCCCC[C@](O)(C(F)(F)F)C2=NN=C(O2)c2nc(c(C(F)(F)F)cc2N)N1]
or an equivalent amount of a deuterated derivative or a pharmaceutically acceptable salt thereof daily.

2. The method according to claim 1, wherein the method comprises administering about 1 mg to about 400 mg of Compound I or an equivalent amount of a deuterated derivative or a pharmaceutically acceptable salt thereof daily

3. The method according to claim 1 or claim 2, wherein the method comprises administering about 5 mg to about 375 mg of Compound I or an equivalent amount of a deuterated derivative or a pharmaceutically acceptable salt thereof daily.

4. The method according to any one of claims 1-3, wherein the method comprises administering about 30 mg to about 300 mg of Compound I or an equivalent amount of a deuterated derivative or a pharmaceutically acceptable salt thereof daily.

5. The method according to any one of claims 1-4, wherein the method comprises administering about 50 mg to about 200 mg of Compound I or an equivalent amount of a deuterated derivative or a pharmaceutically acceptable salt thereof daily.

6. The method according to any one of claims 1-5, wherein the method comprises administering about 50 mg to about 175 mg of Compound I or an equivalent amount of a deuterated derivative or a pharmaceutically acceptable salt thereof daily.

7. The method according to any one of claims 1-3, wherein the method comprises administering about 10 mg to about 200 mg of Compound I or an equivalent amount of a deuterated derivative or a pharmaceutically acceptable salt thereof daily.

8. The method according to any one of claim 1-3 or 7, wherein the method comprises administering about 20 mg to about 100 mg of Compound I or an equivalent amount of a deuterated derivative or a pharmaceutically acceptable salt thereof daily

9. The method according to any one of claim 1-3, 7, or 8, wherein the method comprises administering about 30 mg to about 60 mg of Compound I or an equivalent amount of a deuterated derivative or a pharmaceutically acceptable salt thereof daily

10. The method according to claim 1 or claim 2, wherein the method comprises administering about 1 mg to about 50 mg of Compound I or an equivalent amount of a deuterated derivative or a pharmaceutically acceptable salt thereof daily.

11. The method according to any one of claim 1, 2, or 10, wherein the method comprises administering about 2 mg to about 40 mg of Compound I or an equivalent amount of a deuterated derivative or a pharmaceutically acceptable salt thereof daily.

12. The method according to any one of claim 1-3, or 10, wherein the method comprises administering about 5 mg to about 50 mg of Compound I or an equivalent amount of a deuterated derivative or a pharmaceutically acceptable salt thereof daily.

13. The method according to any one of claim 1-3 or 10-12, wherein the method comprises administering about 5 mg to about 20 mg of Compound I or an equivalent amount of a deuterated derivative or a pharmaceutically acceptable salt thereof daily.

14. The method according to any one of claim 1-3 or 10-13, wherein the method comprises administering about 5 mg to about 10 mg of Compound I or an equivalent amount of a deuterated derivative or a pharmaceutically acceptable salt thereof daily.

15. The method according to any one of claim 1, 2 or 10, wherein the method comprises administering about 1 mg to about 30 mg of Compound I or an equivalent amount of a deuterated derivative or a pharmaceutically acceptable salt thereof daily.

16. The method according to any one of claim 1-4 or 7-9, wherein about 30 mg, about 31 mg, about 32 mg, about 33 mg, about 34 mg, about 35 mg, about 36 mg, about 37 mg, about 38 mg, about 39 mg, about 40 mg, or about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, or about 60 mg of Compound I or an equivalent amount of a deuterated derivative or a pharmaceutically acceptable salt thereof is administered daily.

17. The method according to any one of claims 1-16 wherein the patient is heterozygous and has one F508del mutation.

18. The method according to any one of claims 1-17, wherein Compound I or a deuterated derivative or a pharmaceutically acceptable salt thereof is administered as a single dose, once daily.

19. The method according to any one of claims 1-17, wherein Compound I or a deuterated derivative or a pharmaceutically acceptable salt thereof is administered in two doses daily.

20. The method according to any one of claims 1-19, further comprising administering one or more additional therapeutic agent(s).

21. The method according to claim 20, wherein the one or more additional therapeutic agent(s) comprise(s) a compound with CFTR-modulating activity or a deuterated derivative or a pharmaceutically acceptable salt thereof.

22. The method according to claim 20 or claim 21, wherein the one or more additional therapeutic agent(s) comprise(s) a CFTR corrector.

23. The method according to any one of claims 20-22, wherein the one or more additional therapeutic agent(s) comprise(s) a compound selected from:
(a) (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1/-indol-5-yl) cyclopropanecarboxamide (Compound II): 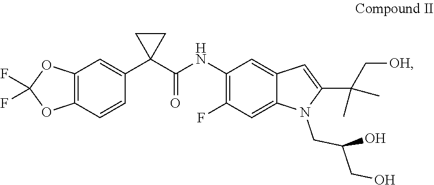 [SMILES: CC(C)(CO)c1cc2cc(NC(=O)C3(c4ccc5c(c4)OC(F)(F)O5)CC3)c(F)cc2n1C[C@@H](O)CO]
(b) 3-(6-(1-(2,2-difluorobenzo[d][1,3]dioxol-5-yl) cyclopropanecarboxamido)-3-methylpyridin-2-yl)benzoic acid (Compound IV): 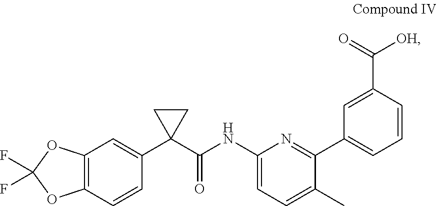 [SMILES: Cc1ccc(NC(=O)C2(c3ccc4c(c3)OC(F)(F)O4)CC2)nc1-c1cccc(C(=O)O)c1]
(c) N-(1,3-dimethylpyrazol-4-yl)sulfonyl-6-[3-(3,3,3-trifluoro-2,2-dimethyl-propoxy) pyrazol-1-yl]-2-[(4S)-2,2,4-trimethylpyrrolidin-1-yl]pyridine-3-carboxamide (Compound V): 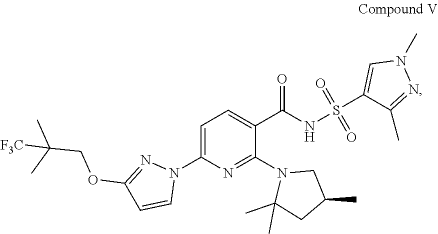 [SMILES: Cc1nn(C)cc1S(=O)(=O)NC(=O)c1ccc(-n2ccc(OCC(C)(C)C(F)(F)F)n2)nc1N1C[C@@H](C)CC1(C)C]
(d) N-(benzenesulfonyl)-6-[3-[2-[1-(trifluoromethyl)cyclopropyl]ethoxy]pyrazol-1-yl]-2-[(4S)-2,2,4-trimethylpyrrolidin-1-yl]pyridine-3-carboxamide (Compound VI): 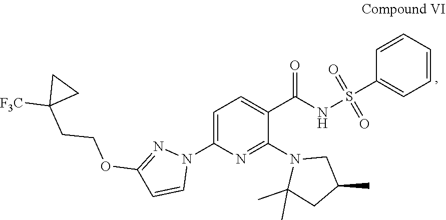 [SMILES: C[C@@H]1CN(c2nc(-n3ccc(OCCC4(C(F)(F)F)CC4)n3)ccc2C(=O)NS(=O)(=O)c2ccccc2)C(C)(C)C1]
(e) (14S)-8-[3-(2-{dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1 (22),5,7,9,19 (23),20-hexaene-2,2,4-trione (Compound VII): 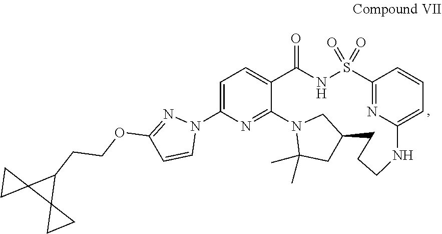 [SMILES: CC1(C)C[C@@H]2CCCNc3cccc(n3)S(=O)(=O)NC(=O)c3ccc(-n4ccc(OCCC5C6(CC6)C56CC6)n4)nc3N1C2]
(f) (11R)-6-(2,6-dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1 (17),4 (19),5,7,14 (18), 15-hexaene-2,2,13-trione (Compound VIII): 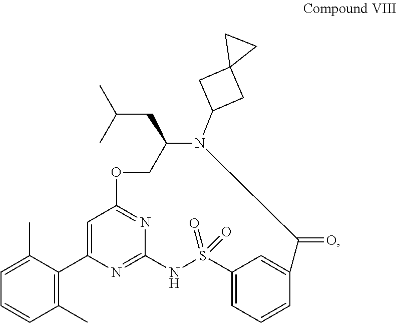 [SMILES: Cc1cccc(C)c1-c1cc2nc(n1)NS(=O)(=O)c1cccc(c1)C(=O)N(C1CC3(CC3)C1)[C@H](CC(C)C)CO2]
(g) N-(benzenesulfonyl)-6-(3-fluoro-5-isobutoxy-phenyl)-2-[(4S)-2,2,4-trimethylpyrrolidin-1-yl]pyridine-3-carboxamide (Compound IX): 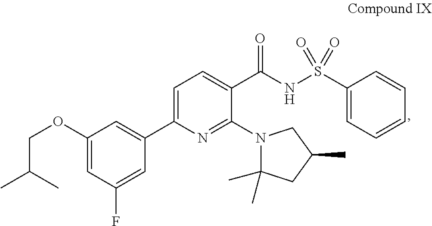 [SMILES: CC(C)COc1cc(F)cc(-c2ccc(C(=O)NS(=O)(=O)c3ccccc3)c(N3C[C@@H](C)CC3(C)C)n2)c1]
and
(h) N-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide (Compound X): 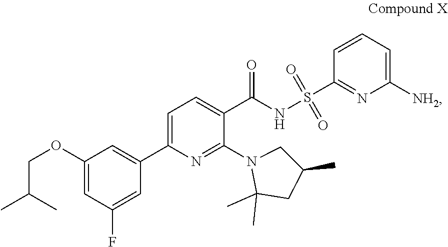 [SMILES: CC(C)COc1cc(F)cc(-c2ccc(C(=O)NS(=O)(=O)c3cccc(N)n3)c(N3C[C@@H](C)CC3(C)C)n2)c1]
and deuterated derivatives and pharmaceutically acceptable salts thereof.

24. The method according to any one of claims 20-23, wherein the one or more additional therapeutic agent(s) comprise(s) at least one compound selected from PTI-428, ABBV-2222, ABBV-2851, GLPG2737, ABBV-3221, ABBV-3748, ABBV-3903, ABBV-119, ABBV-2851, FDL-169, ARN5562, ARN21586, ARN22081, ARN22652, ARN23765, ARN23766, and PTI-801.

25. The method according to any one of claims 20-24, wherein the one or more additional therapeutic agent(s) comprise(s) a CFTR potentiator enhancer.

26. The method according to any one of claims 20-25, wherein the one or more additional therapeutic agent(s) comprise(s) ASP-11.

27. The method according to any one of E claims 20-26, wherein the one or more additional therapeutic agent(s) comprise(s) a CFTR potentiator.

28. The method according to any one of claims 20-27, wherein the one or more additional therapeutic agent(s) comprise(s) a compound selected from:
(c) N-(5-hydroxy-2,4-di-tert-butyl-phenyl)-4-oxo-1H-quinoline-3-carboxamide (Compound III): 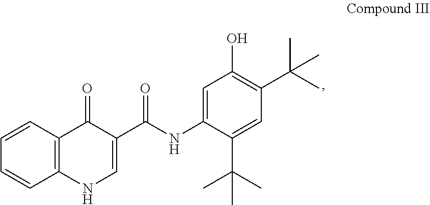 [SMILES: CC(C)(C)c1cc(C(C)(C)C)c(NC(=O)c2c[nH]c3ccccc3c2=O)cc1O]
and
(d) N-(2-(tert-butyl)-5-hydroxy-4-(2-(methyl-d3) propan-2-yl-1,1,1,3,3,3-d6)phenyl)-4-oxo-1,4-dihydroquinoline-3-carboxamide (Compound III-d): 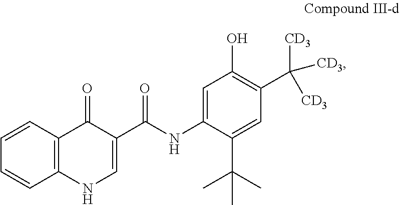 [SMILES: [2H]C([2H])([2H])C(c1cc(C(C)(C)C)c(NC(=O)c2c[nH]c3ccccc3c2=O)cc1O)(C([2H])([2H])[2H])C([2H])([2H])[2H]]
and deuterated derivatives and pharmaceutically acceptable salts thereof.

29. The method according to any one of claims 20-28, wherein the one or more additional therapeutic agent(s) comprise(s) at least one compound selected from FDL-176, PTI-808, GLPG1837/ABBV-974, GLPG2451/ABBV-2451, QBW251 (Icenticaftor), GLPG3067/ABBV-3067 (Navocaftor), and ABBV-191.

30. The method according to any one of claims 20-29, wherein the one or more additional therapeutic agent(s) comprise(s) a CFTR amplifier.

31. The method according to any one of claims 20-30, wherein the one or more additional therapeutic agent(s) comprise(s) PTI-428.

32. The method according to any one of claims 20-31, wherein the one or more additional therapeutic agent(s) comprise(s) a CFTR readthrough agent.

33. The method according to any one of claims 20-32, wherein the one or more additional therapeutic agent(s) comprise(s) ELX-02.

34. The method according to any one of claims 20-33, wherein the one or more additional therapeutic agent(s) comprise(s) a nucleic acid therapy.

35. The method according to any one of claims 20-34, wherein the one or more additional therapeutic agent(s) comprise(s) at least one agent selected from MRT5005, Lunar-CF, and RCT223.

36. The method according to any one of claims 20-35, wherein the one or more additional therapeutic agent(s) comprise(s) an ENaC inhibitor.

37. The method according to any one of claims 20-36, wherein the one or more additional therapeutic agent(s) comprise(s) amiloride, ETD001, CF552, GS-9411, GS-5737, P-1037 (VX-371), P-1055 (VX-551), AZD5634, SPX-101, Ionis-ENaC-2.5 Rx, BI 1265162, or ARO-ENaC1001.

38. The method according to any one of claims 20-37, wherein the one or more additional therapeutic agent(s) comprise(s) a TMEM16A modulator.

39. The method according to any one of claims 20-38, wherein the one or more additional therapeutic agent(s) comprise(s) ETD002.

40. The method according to any one of claims 20-39, wherein the one or more additional therapeutic agent(s) comprise(s) a GPR39 agonist.

41. The method according to any one of claims 20-40, wherein the one or more additional therapeutic agent(s) comprise(s) DS-1039.

42. A pharmaceutical composition comprising about 0.1 mg to about 800 mg of Compound I or an equivalent amount of a deuterated derivative or a pharmaceutically acceptable salt thereof.

43. The pharmaceutical composition according to claim 42, wherein the composition comprises about 1 mg to about 400 mg of Compound I or an equivalent amount of a deuterated derivative or a pharmaceutically acceptable salt thereof.

44. The pharmaceutical composition according to claim 42 or claim 43, wherein the composition comprises about 5 mg to about 375 mg of Compound I or an equivalent amount of a deuterated derivative or a pharmaceutically acceptable salt thereof.

45. The pharmaceutical composition according to any one of claims 42-44, wherein the composition comprises about 30 mg to about 300 mg of Compound I or an equivalent amount of a deuterated derivative or a pharmaceutically acceptable salt thereof.

46. The pharmaceutical composition according to any one of claims 42-45, wherein the composition comprises about 50 mg to about 200 mg of Compound I or an equivalent amount of a deuterated derivative or a pharmaceutically acceptable salt thereof.

47. The pharmaceutical composition according to any one of claims 42-46, wherein the composition comprises about 50 mg to about 175 mg of Compound I or an equivalent amount of a deuterated derivative or a pharmaceutically acceptable salt thereof.

48. The pharmaceutical composition according to any one of claims 42-44, wherein the composition comprises about 10 mg to about 200 mg of Compound I or an equivalent amount of a deuterated derivative or a pharmaceutically acceptable salt thereof.

49. The pharmaceutical composition according to any one of claim 42-44 or 48, wherein the composition comprises about 20 mg to about 100 mg of Compound I or an equivalent amount of a deuterated derivative or a pharmaceutically acceptable salt thereof.

50. The pharmaceutical composition according to any one of claim 42-44, 48, or 49, wherein the composition comprises about 30 mg to about 60 mg of Compound I or an equivalent amount of a deuterated derivative or a pharmaceutically acceptable salt thereof.

51. The pharmaceutical composition according to claim 42 or claim 43, wherein the composition comprises about 1 mg to about 50 mg of Compound I or an equivalent amount of a deuterated derivative or a pharmaceutically acceptable salt thereof.

52. The pharmaceutical composition according to any one of claim 42, 43, or 51, wherein the composition comprises about 2 mg to about 40 mg of Compound I or an equivalent amount of a deuterated derivative or a pharmaceutically acceptable salt thereof.

53. The pharmaceutical composition according to any one of claim 42-44, or 51, wherein the composition comprises about 5 mg to about 50 mg of Compound I or an equivalent amount of a deuterated derivative or a pharmaceutically acceptable salt thereof.

54. The pharmaceutical composition according to any one of claim 42-44, or 51-53, wherein the composition comprises about 5 mg to about 20 mg of Compound I or an equivalent amount of a deuterated derivative or a pharmaceutically acceptable salt thereof.

55. The pharmaceutical composition according to any one of claim 42-44, or 51-54, wherein the composition comprises about 5 mg to about 10 mg of Compound I or an equivalent amount of a deuterated derivative or a pharmaceutically acceptable salt thereof.

56. The pharmaceutical composition according to any one of claim 42, 43, or 51, wherein the composition comprises about 1 mg to about 30 mg of Compound I or an equivalent amount of a deuterated derivative or a pharmaceutically acceptable salt thereof.

57. The pharmaceutical composition according to any one of claim 42-45, or 48-50, wherein the composition comprises about 30 mg, about 31 mg, about 32 mg, about 33 mg, about 34 mg, about 35 mg, about 36 mg, about 37 mg, about 38 mg, about 39 mg, about 40 mg, or about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, or about 60 mg of Compound I or an equivalent amount of a deuterated derivative or a pharmaceutically acceptable salt thereof.

58. The pharmaceutical composition according to any one of claims 42-57, wherein the composition further comprises one or more additional therapeutic agent(s).

59. The pharmaceutical composition according to claim 58, wherein the one or more additional therapeutic agent(s) comprise(s) a compound with CFTR-modulating activity or a deuterated derivative or a pharmaceutically acceptable salt thereof.

60. The pharmaceutical composition according to claim 58 or claim 59, wherein the one or more additional therapeutic agent(s) comprise(s) a CFTR corrector.

61. The pharmaceutical composition according to any one of claims 58-60, wherein the one or more additional therapeutic agent(s) comprise(s) a compound selected from:
(a) Compound II,
(b) Compound IV,
(c) Compound V,
(d) Compound VI,
(e) Compound VII,
(f) Compound VIII,
(g) Compound IX, and
(i) Compound X, and deuterated derivatives and pharmaceutically acceptable salts thereof.

62. The pharmaceutical composition according to any one of claims 58-61, wherein the one or more additional therapeutic agent(s) comprise(s) at least one compound selected from PTI-428, ABBV-2222, ABBV-2851, GLPG2737, ABBV-3221, ABBV-3748, ABBV-3903, ABBV-119, ABBV-2851, FDL-169, ARN5562, ARN21586, ARN22081, ARN22652, ARN23765, ARN23766, and PTI-801.

63. The pharmaceutical composition according to any one of claims 58-62, wherein the one or more additional therapeutic agent(s) comprise(s) a CFTR potentiator enhancer.

64. The pharmaceutical composition according to any one of claims 58-63, wherein the one or more additional therapeutic agent(s) comprise(s) ASP-11.

65. The pharmaceutical composition according to any one of claims 58-64, wherein the one or more additional therapeutic agent(s) comprise(s) a CFTR potentiator.

66. The pharmaceutical composition according to any one of claims 58-65, wherein the one or more additional therapeutic agent(s) comprise(s) a compound selected from Compound III, Compound III-d, and deuterated derivatives and pharmaceutically acceptable salts thereof.

67. The pharmaceutical composition according to any one of claims 58-66, wherein the one or more additional therapeutic agent(s) comprise(s) at least one compound selected from FDL-176, PTI-808, GLPG1837/ABBV-974, GLPG2451/ABBV-2451, QBW251 (Icenticaftor), GLPG3067/ABBV-3067 (Navocaftor), and ABBV-191.

68. The pharmaceutical composition according to any one of claims 58-67, wherein the one or more additional therapeutic agent(s) comprise(s) a CFTR amplifier.

69. The pharmaceutical composition according to any one of claims 58-68, wherein the one or more additional therapeutic agent(s) comprise(s) PTI-428.

70. The pharmaceutical composition according to any one of claims 58-69, wherein the one or more additional therapeutic agent(s) comprise(s) a CFTR readthrough agent.

71. The pharmaceutical composition according to any one of claims 58-70, wherein the one or more additional therapeutic agent(s) comprise(s) ELX-02.

72. The pharmaceutical composition according to any one of claims 58-71, wherein the one or more additional therapeutic agent(s) comprise(s) a nucleic acid therapy.

73. The pharmaceutical composition according to any one of claims 58-72, wherein the one or more additional therapeutic agent(s) comprise(s) at least one agent selected from MRT5005, Lunar-CF, and RCT223.

74. The pharmaceutical composition according to any one of claims 58-73, wherein the one or more additional therapeutic agent(s) comprise(s) an ENaC inhibitor.

75. The pharmaceutical composition according to any one of claims 58-74, wherein the one or more additional therapeutic agent(s) comprise(s) amiloride, ETD001, CF552, GS-9411, GS-5737, P-1037 (VX-371), P-1055 (VX-551), AZD5634, SPX-101, Ionis-ENaC-2.5 Rx, BI 1265162, or ARO-ENaC1001.

76. The pharmaceutical composition according to any one of claims 58-75, wherein the one or more additional therapeutic agent(s) comprise(s) a TMEM16A modulator.

77. The pharmaceutical composition according to any one of claims 58-76, wherein the one or more additional therapeutic agent(s) comprise(s) ETD002.

78. The pharmaceutical composition according to any one of claims 58-77, wherein the one or more additional therapeutic agent(s) comprise(s) a GPR39 agonist.

79. The pharmaceutical composition according to any one of claims 58-78, wherein the one or more additional therapeutic agent(s) comprise(s) DS-1039.

80. Compound I or a deuterated derivative or pharmaceutically acceptable salt thereof, or a pharmaceutical composition according to any one of claims 42-79, for use in a method of treating cystic fibrosis.

81. Use of Compound I or a deuterated derivative or pharmaceutically acceptable salt thereof, or a pharmaceutical composition according to any one of claims 42-79, in the manufacture of a medicament for treating cystic fibrosis.
